(19) United States
(12) Patent Application Publication
(10) Pub. No.: US 2025/0302996 A1
(43) Pub. Date: Oct. 2, 2025

(54) METHOD

(71) Applicant: Oxford University Innovation Limited, Oxford (GB)

(72) Inventors: Carlo RINALDI, Oxford (GB); Matthew WOOD, Oxford (GB); Muhammad HANIFI, Oxford (GB)

(21) Appl. No.: 18/865,508

(22) PCT Filed: May 12, 2023
(86) PCT No.: PCT/GB2023/051256

§ 371 Date: Nov. 13, 2024

(30) Foreign Application Priority Data
May 13, 2022 (GB) 2207026.2

(51) Int. Cl. A61K 48/00; A61K 38/17; A61P 21/00; C12N 9/22; C12N 15/11; C12N 15/86

(52) U.S. Cl. CPC A61K 48/005; A61K 38/1709; A61P 21/00; C12N 9/226; C12N 15/111; C12N 15/86; C12N 2310/20; C12N 2320/33; C12N 2740/15043; C12N 2750/14143

(57) ABSTRACT

The invention relates to modulating the function of a regulatory element in a nucleic acid, comprising delivering to a cell a catalytically inactive Cas13 (dCas13) protein and a CRISPR RNA (crRNA), wherein the crRNA recruits the dCas13 protein to the regulatory element, such that the dCas13 protein sterically blocks the regulatory element.

### FIELD OF INVENTION

The invention relates to compounds for modulating the function of a regulatory element in a nucleic acid, and methods and uses thereof.

### BACKGROUND OF THE INVENTION

RNA molecules are comprised of regulatory elements, and the functions of these regulatory elements can be modulated through the use of target-specific gene modulating technologies.

For example, antisense oligonucleotides (ASOs) have been developed to degrade target RNAs or to act as steric blockers for various applications. For example, ASOs can be used to induce splice-switching, inhibit translation initiation, interfere with upstream open reading frames (uORFs) that negatively regulate translation, inhibit nonsense-mediated decay by preventing assembly of exon junction complexes, and influence polyadenylation signals to increase transcript stability. However, translation into clinical applications has been seriously hampered by issues related to off-target effects, inadequate target engagement and poor delivery to target tissues.

In recent years, the type VI CRISPR-Cas13 system which recognise, bind and degrade target RNAs, has been harnessed in various contexts for RNA manipulation. For example, Zhang et al. (2020) used a CRISPR-Cas13a based strategy that tracks and degrades toxic RNA in myotonic dystrophy type 1 (Reference 1). Cox et al. (2017) generated a fusion protein between a catalytically inactive PspCas13b and the ADAR deaminase domain for targeted gene-editing (Reference 2). However, these systems result in the degradation of the target RNA or alteration of the target RNA sequence, and have undesirable off-target effects.

It is an object of the invention to identify further and improve compounds and methods for modulating the function of a regulatory element in a nucleic acid, in particular, without degrading or editing the target RNA.

### SUMMARY OF THE INVENTION

The inventors identified a new way of modulating the function of a regulatory element in a nucleic acid. In particular, the inventors found that a catalytically inactive Cas13 (dCas13) protein, when recruited to a regulatory element in a nucleic acid by a CRISPR RNA (crRNA), can act as a steric blocker at the target site, thereby modulating the function of the regulatory element.

The inventors identified Psp-dCas13b as a potent steric blocker. When programmed using crRNA to target an RNA sequence, the inventors found that Psp-dCas13b is capable of blocking the target RNA sequence from interacting with ribonucleoprotein complexes such as ribosomes and miRNA silencing complexes. In addition, the inventors found that this steric blocking capacity is (a) enhanced when the native length of the crRNA spacer sequence is reduced to around 18-24 nucleotides; and (b) unaffected by C-terminal truncation of the Psp-dCas13b. Utilising a crRNA with a 21-nucleotide spacer length and a C-terminally truncated variant of Psp-dCas13b, the inventors demonstrate that Psp-dCas13b can reverse deregulated splicing in both DM1 patient-derived cells and a DM1 mouse model. Collectively, these observations show that the present invention may be useful in treating diseases including, but not limited to, repeat expansion diseases (e.g., DM1, DM2, Fuchs endothelial corneal dystrophy), diseases where miRNA dysregulation plays a role in the pathology, diseases where translation dysregulation plays a role in the pathology (e.g., Huntington's disease, amyotrophic lateral sclerosis), and RNA mis-splicing diseases (e.g., spinal muscular atrophy, Duchenne muscular dystrophy).

Compared to small RNAs and RNA interference, which are difficult in design and are limited by high off-target effects, CRISPR/dCas13 can be used to manipulate only the target RNA, with few or no off-target effects in eukaryotes, and multiple crRNAs can be used to manipulate a particular mRNA transcript.

All Cas13 proteins are RNA-targeting and so have potential to be repurposed into a dCas13 steric blocker. However, it is unknown which family of Cas13 proteins can achieve the highest steric blocking efficiency when repurposed into a dCas13 steric blocker. Therefore, the inventors first compared the steric blocking efficiency between three dCas13 orthologues Lwa-dCas13a, Psp-dCas13b, and Rfx-dCas13d. In an assay where each orthologue was programmed to target various regulatory sequences in a bidirectional fluorescent reporter transgene, the inventors found that Psp-dCas13b is the most potent blocker of both ribosomes and miRNA-associated complexes (FIG. 1).

To further improve the steric blocking efficiency of Psp-dCas13b, the inventors tested different lengths of crRNA spacer and found that spacers of 18-24 nt (which are shorter than the native length of 30 nt) mediate a surprisingly higher blocking efficiency (FIG. 2A). Fluorescent reporter experiments then confirmed that the steric blocking effect observed requires both Psp-dCas13b and crRNA and not each component alone (see FIG. 2B).

To explore the general applicability of Psp-dCas13b for blocking miRNA functions, the inventors then programmed Psp-dCas13b to target three different miRNA binding sites encoded at the 3′ UTR of a fluorescent reporter transgene. In this assay, the inventors showed that Psp-dCas13b can block all three miRNA binding sites when programmed to partially or completely target the binding site sequence (FIG. 3A). In addition, the inventors observed that the de-repression efficiency achieved from blocking the miRNA target site with Psp-dCas13b phenocopies ASO-mediated miRNA inhibition (FIG. 3B).

Upon establishing the applicability of Psp-dCas13b to block ribosomal- and miRNA-functions, the inventors explored the utility of the system to treat an RNA-dominant disease type 1 myotonic dystrophy (DM1), which is caused by an expansion of the (CTG)n repeat sequence at the 3′ UTR of the DMPK1 gene. The expanded sequence is subsequently transcribed into a toxic RNA that sequesters cellular splicing factors, leading to widespread spliceopathy (FIG. 4A). The inventors found that Psp-dCas13b, when programmed to target the expanded sequence using crRNA of spacer length 21 nt or 24 nt, can consistently and completely reverse the disease-associated splicing pattern across six biomarker exons in DM1 patient-derived muscle cells (FIG. 4B-F).

The inventors prepared a truncated version of Psp-dCas13b that not only has a steric blocking efficiency similar to that of the full-length version but also is small enough to be encoded by a single AAV vector (e.g., AAV9 vector). In particular, the inventors prepared two C-terminally truncated variants of Psp-dCas13b and observed in a fluorescent reporter assay that both of the truncated variants (one of 1053 amino acid residues in length [1053-aa] and one of 984 amino acid residues in length [984-aa]) have comparable ribosomal-blocking efficiencies and sensitivities to spacer length with the full-length variant (FIG. 6).

The inventors then packaged an AAV9 vector encoding a pCMV-driven 984-aa variant (mini-Psp-dCas13b) and a phU6-driven 21-nt-spacer crRNA targeting the expanded (CTG)n repeat sequence. When injected into a mouse model of DM1, the mini-Psp-dCas13b was found to reverse splicing deregulation (FIG. 7).

Accordingly, the invention provides a method of modulating the function of a regulatory element in a target RNA, comprising delivering to a cell a dCas13 and a crRNA, wherein the crRNA recruits the dCas13 protein to the regulatory element, such that the function of the regulatory element is modulated.

The invention also provides a method of modulating the availability, expression and/or activity of a nucleic acid or protein of interest, comprising modulating the function of a regulatory element in a target RNA according to the method of the invention, wherein the target RNA encodes or regulates the nucleic acid or protein of interest, such that the availability, expression and/or activity of the nucleic acid or protein of interest is increased or decreased.

The invention also provides a method of blocking a miRNA-binding site, comprising modulating the function of a regulatory element in a target RNA according to the method of the invention, or modulating the availability, expression and/or activity of a nucleic acid or protein of interest according to the method of the invention, wherein the crRNA comprises a spacer that is complementary to the miRNA-binding site, such that miRNA-mediated silencing of the target RNA is reduced.

The invention also provides a method of blocking ribosomal attachment or translation, comprising modulating the function of a regulatory element in a target RNA according to the method of the invention, or modulating the availability, expression and/or activity of a nucleic acid or protein of interest according to the method of the invention, wherein the crRNA comprises a spacer that is complementary to a start codon of an open reading frame or a start codon of an upstream open reading frame (uORF), such that translation of the target RNA is reduced.

The invention also provides a method of inducing splice switching, comprising modulating the function of a regulatory element in a target RNA according to the method of the invention, or modulating the availability, expression and/or activity of a nucleic acid or protein of interest according to the method of the invention, wherein the crRNA comprises a spacer that is complementary to a splicing element, such as a RNP-binding site, such that splicing of the target RNA is modulated.

The invention also provides a crRNA specific for dCas13b comprises: (i) a dCas13b-specific direct repeat, and (ii) a spacer which is capable of specifically hybridizing with the target RNA sequence and having length of between 18 to 24 nucleotides.

The invention also provides a dCas13b protein consisting of an amino acid sequence having a sequence identity of ≥85% to SEQ ID NO: 2 or 3, provided that the amino acid residues corresponding to the amino acid residue at position 133 of SEQ ID NO: 2 or 3 is alanine.

The invention also provides a polynucleotide or a vector encoding the crRNA according to the invention or the dCas13b protein according to the invention, optionally wherein the vector is AAV or lentivirus.

The invention also provides a delivery vehicle comprising the crRNA according to the invention, the dCas13b protein according to the invention, or the polynucleotide or a vector according to the invention.

The invention also provides a pharmaceutical composition comprising: (i) the crRNA according to the invention or the dCas13b protein according to the invention, (ii) and a pharmaceutically acceptable carrier.

The invention also provides the crRNA or the dCas13b protein according to the invention for use in a method of therapy practised on the human or animal body.

The invention also provides the crRNA or the dCas13b protein according to the invention for use in the method of treating a repeat expansion disease, optionally wherein the expansion disease is type 1 myotonic dystrophy (DM1), myotonic dystrophy type 2 or Fuchs endothelial corneal dystrophy.

The invention also provides a method of treating or preventing a repeat expansion disease in a subject, wherein the method comprises administering to a subject a therapeutically effective amount of a dCas13 protein and crRNA, wherein the method comprises modulating the function of a regulatory element in a nucleic acid according to the method of the invention, or modulating the availability, expression and/or activity of a nucleic acid or protein of interest according to the method of the invention, wherein the crRNA comprises a spacer complementary to an expanded repeat sequence, and optionally wherein the expansion disease is type 1 myotonic dystrophy (DM1), myotonic dystrophy type 2 or Fuchs endothelial corneal dystrophy.

The invention also provides a dCas13 protein for use in a method of treating a repeat expansion disease, wherein the method comprises administering to a subject the dCas13 protein and a crRNA, wherein the crRNA comprises a spacer complementary to an expanded repeat sequence, and optionally wherein the expansion disease is type 1 myotonic dystrophy (DM1), myotonic dystrophy type 2 or Fuchs endothelial corneal dystrophy.

The invention also provides a dCas13 protein for use in a method of treating a repeat expansion disease, wherein the method comprises administering to a subject therapeutically effective amount of the dCas13 protein and a crRNA, wherein the method comprises modulating the function of a regulatory element in a nucleic acid according to the method of the invention, or modulating the availability, expression and/or activity of a nucleic acid or protein of interest according to the method of the invention, wherein the crRNA comprises a spacer complementary to an expanded repeat sequence, and optionally wherein the expansion disease is type 1 myotonic dystrophy (DM1), myotonic dystrophy type 2 or Fuchs endothelial corneal dystrophy.

The invention also provides use of the crRNA or the dCas13b protein according to the invention in the preparation of a medicament for a method of treating a repeat expansion disease, optionally wherein the expansion disease is type 1 myotonic dystrophy (DM1), myotonic dystrophy type 2 or Fuchs endothelial corneal dystrophy.

### BRIEF DESCRIPTION OF THE FIGURES

FIG. 1 shows that Psp-dCas13b can be repurposed into an efficient RNA steric blocker. A) Schematic diagram of the design of the ribosomal- and miRNA-blocking experiments. HEK293T cells were co-transfected with plasmids encoding Lwa-dCas13a, Psp-dCas13b, or Rfx-dCas13d, plasmids encoding the corresponding crRNA, and plasmids encoding a bidirectional reporter expressing ECFP and mIFP. In the ribosomal-blocking experiment, the dCas13 orthologues were targeted by the corresponding crRNA to bind the 5′ UTR or TSS of the ECFP transcript (top right). In the miRNA-blocking experiment, the dCas13b orthologues were targeted by the corresponding crRNA to bind the miR-17 binding site at the 3′ UTR of the ECFP transcript (bottom right). B) and C) Plots comparing the blocking efficiency of Lwa-dCas13a, Psp-dCas13b, and Rfx-dCas13d when targeted by the corresponding crRNA to bind various sites of the ECFP transcript. Six crRNAs were tested for each dCas13b orthologue, three of which targeted the 5′ UTR or TSS of the ECFP transcript and three of which targeted the miR-17 binding site of the ECFP transcript. The expression levels of ECFP were quantified by flow cytometry and normalized to those of mIFP. P values are generated from two-tailed Student's t-test.

FIG. 2 shows that shortening the crRNA spacer improves the steric blocking efficiency of Psp-dCas13b. The expression levels of ECFP were quantified by flow cytometry and normalized to those of mIFP. A) The effect of varying the spacer length of three different crRNAs targeting the 5′ UTR or TSS of the ECFP transcript. Shorter spacer sequences of 18-24 nt exhibited the most efficient knock-down of ECFP expression. B) Plot comparing the blocking efficiency of Psp-dCas13b alone, crRNA alone, and Psp-dCas13b and crRNA combined. Psp-dCas13b and the corresponding crRNA are both necessary to achieve an efficient blocking of ECFP expression. Scr, scrambled non-targeting control.

FIG. 3 shows that Psp-dCas13b can completely and specifically perturb miRNA function. The expression levels of ECFP were quantified by flow cytometry and normalized to those of mIFP. A) Positional effects of crRNA on the de-repression of ECFP expression. A total of 45 crRNAs were designed to guide Psp-dCas13b towards the target site of miR-17, miR-92a, or miR-222 at the 3′ UTR of the ECFP transcript. B) Plot comparing the extent of Psp-dCas13b-mediated miRNA de-repression against antagomiR-17. Scr, scrambled non-targeting control.

FIG. 4 shows that Psp-dCas13b reverses pathological splicing patterns in a DM1 cell model. Scr, scrambled non-targeting crRNA. A) is a schematic of DM1 pathology. The wild-type DMPK1 allele contains 5-37 CTG repeats, while in DM1 mutants, up to 4,000 repeats can be found. B) Schematic diagram of the plasmid encoding NLS-dCas13b-NLS-P2A-EGFP and crRNA sequence. Five different crRNAs with different spacer lengths were designed. C) RT-PCR and gel analysis demonstrating reversal of spliceopathy in DM1 cells. Schematic diagrams of exon exclusions and exon inclusions are provided to the right of the gel images. D) Quantification of the gel images shown in C) by densitometry demonstrating a complete reversal of splicing pattern in five out of six disease biomarkers. E) RNAseq data represented on UCSC genome-browser tracks of TEAD1 exon 5 and KIF13A exon 38, showing DM1-related mis-splicing events in myotubes from unaffected individuals (WT) or DM1 patients (DM1), either untransfected, transfected with dCas13 and scrambled control crRNA, or transfected with dCas13 and crRNA3, as indicated. F) RNAseq data represented on UCSC genome-browser tracks of MBNL1 exon 6, MBNL2 exon 6, and DMD exon 78, showing DM1-related mis-splicing events in myotubes from unaffected individuals (WT) or DM1 patients (DM1), either untransfected, transfected with dCas13 and control crRNA, or transfected with dCas13 with crRNA3, as indicated.

FIG. 5 shows that Psp-dCas13b outperforms antisense oligonucleotides in reversing DM1-related mis-splicing as indicated by RNAseq data. A) and D) are scatterplots showing relative inclusion level (PSI; Ψ) of mis-spliced events in WT or DM1 myotubes transfected with either dCas13b/Scr-ctrl (A) or ASO/ctrl (D). B) and E) are scatterplots showing relative inclusion rate (PSI; Ψ) of mis-spliced events in WT and DM1 myotubes transfected with either dCas13b/crRNA3 (B) or ASO/CAG7 (E). C) and F) are pie charts showing the proportion of mis-spliced events that achieved complete reversal, partial reversal, or no reversal following transfection either with dCas13/crRNA3 (C) or with ASO/CAG7 (F). Scr-ctrl, scrambled non-targeting control crRNA. ASO, antisense oligonucleotide.

FIG. 6 shows that C-terminally truncated Psp-dCas13b retains its steric blocking efficiency. A) Schematic diagram of full-length and C-terminally truncated variants of Psp-dCas13b. B) Graph comparing the blocking efficiency and sensitivity to crRNA spacer length of full-length and truncated variants of Psp-dCas13b. The full-length and truncated variants of Psp-dCas13b were equally effective at blocking and equally sensitive to crRNA spacer length. The expression levels of ECFP were quantified by flow cytometry and normalized to those of mIFP. Scr, scrambled non-targeting crRNA.

FIG. 7 shows that treatment of adult skeletal muscle of the HSA-LR DM1 mouse model with Psp-dCas13b corrects splicing deregulation. Splicing patterns were determined by RT-PCR. A) Schematic diagram showing single-vector administration of Psp-dCas13b and the corresponding crRNA. Either a therapeutic or control vector was injected into the TA of 8-week-old HSALR mice. B) Quantification of splicing pattern of four disease biomarkers four weeks after treatment with pCMV-driven EGFP control or pEFS-driven full-length Psp-dCas13b therapeutic vector (n=3). C) Quantification of splicing pattern of a disease biomarker four weeks after treatment with pCMV-driven EGFP control or pCMV-driven mini-Psp-dCas13b therapeutic vector (n=5).

### BRIEF DESCRIPTION OF THE SEQUENCE LISTING

SEQ ID NO: 1 shows the polypeptide sequence of Psp-dCas13b.

SEQ ID NO: 2 shows the polypeptide sequence of the C-terminally truncated variant of Psp-dCas13b of 984 amino acid residues in length (984-aa), i.e., mini-Psp-dCas13b.

SEQ ID NO: 3 shows the polypeptide sequence of the C-terminally truncated variant of Psp-dCas13b of 1053 amino acid residues in length.

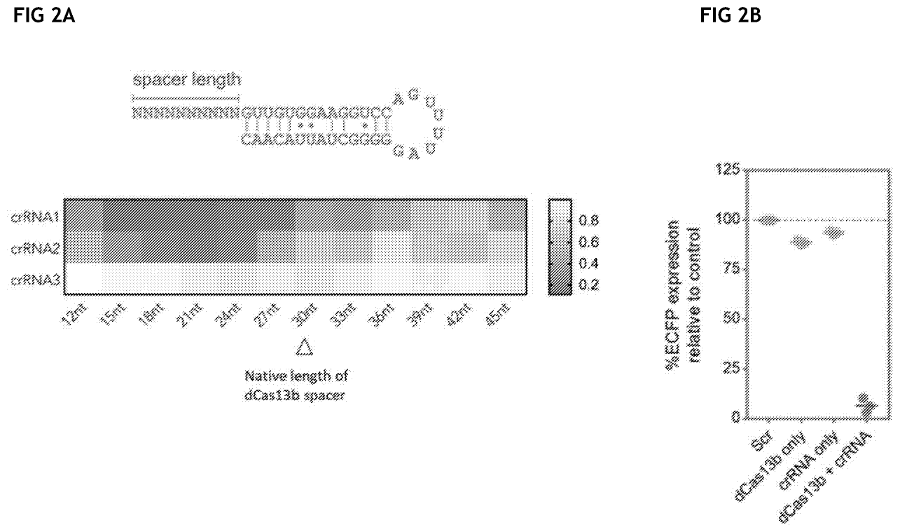

SEQ ID NO: 4 shows the polyribonucleotide sequence of the crRNA compatible with Psp-dCas13b shown in FIG. 2A.

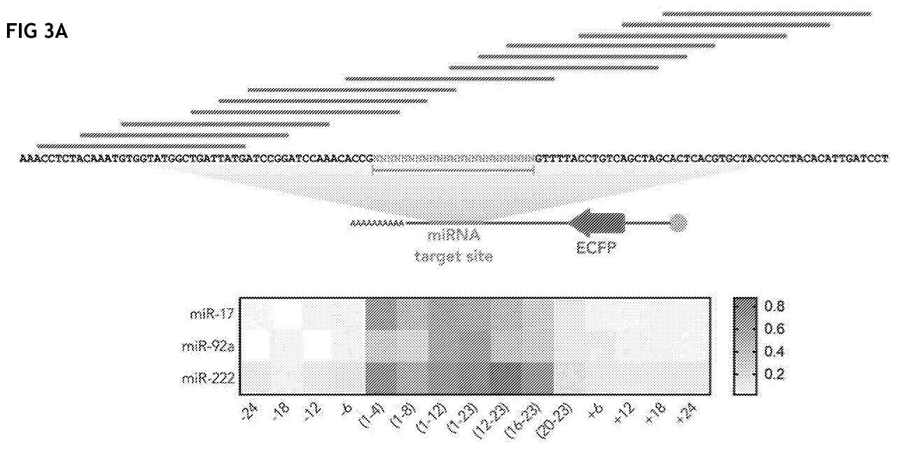

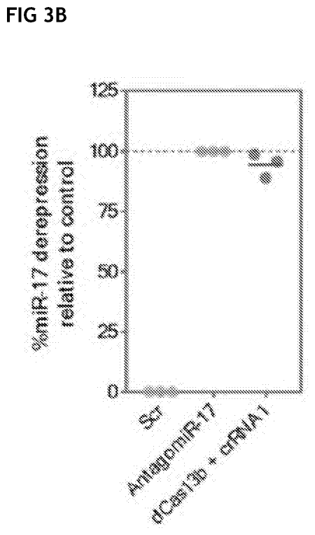

SEQ ID NO: 5 shows the polynucleotide sequence of the ECFP transgene containing an miRNA target site shown in FIG. 3A.

SEQ ID NO: 6 shows the poly(A) tail sequence of the ECFP transgene shown in FIG. 3A.

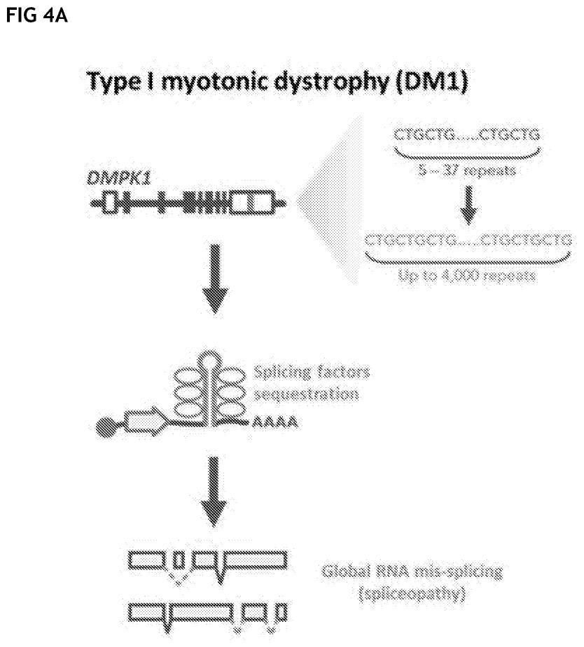

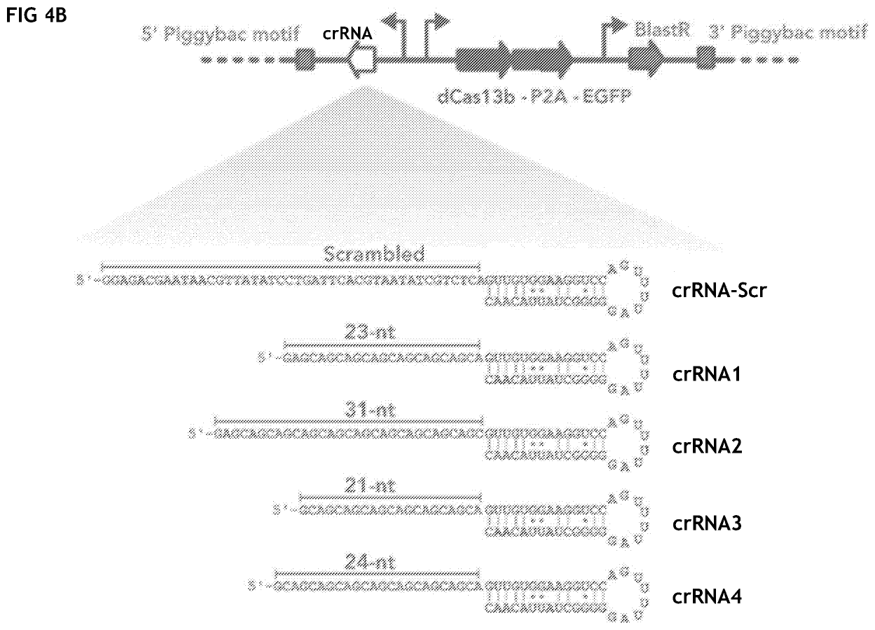

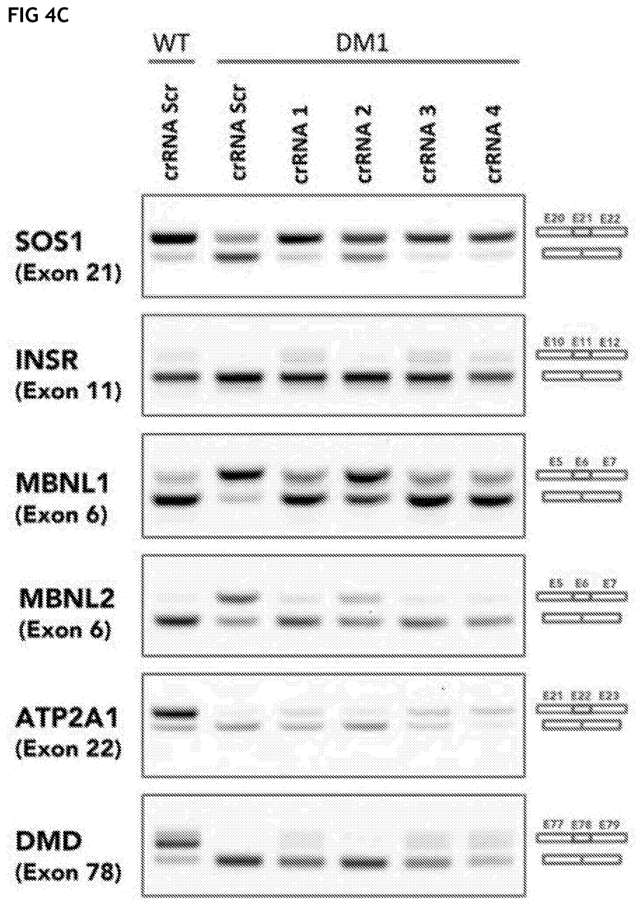

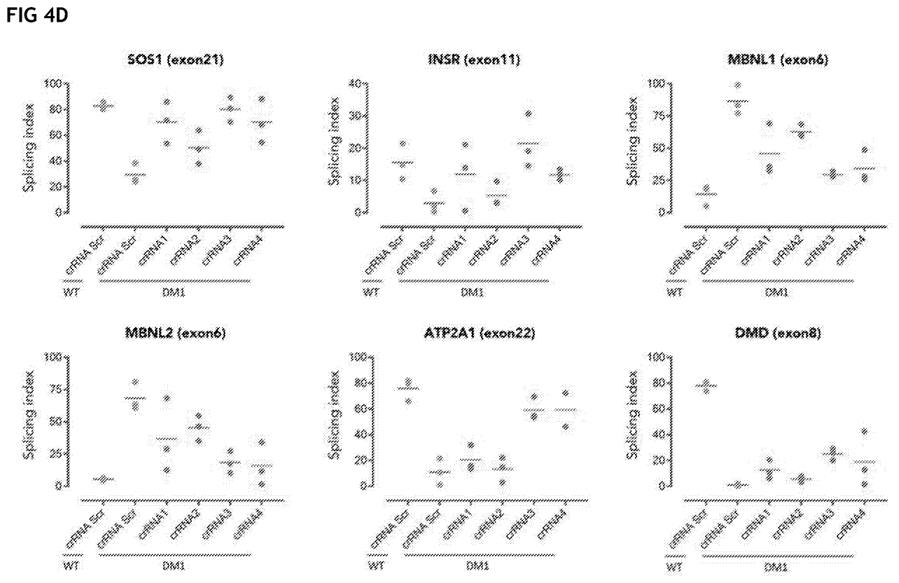

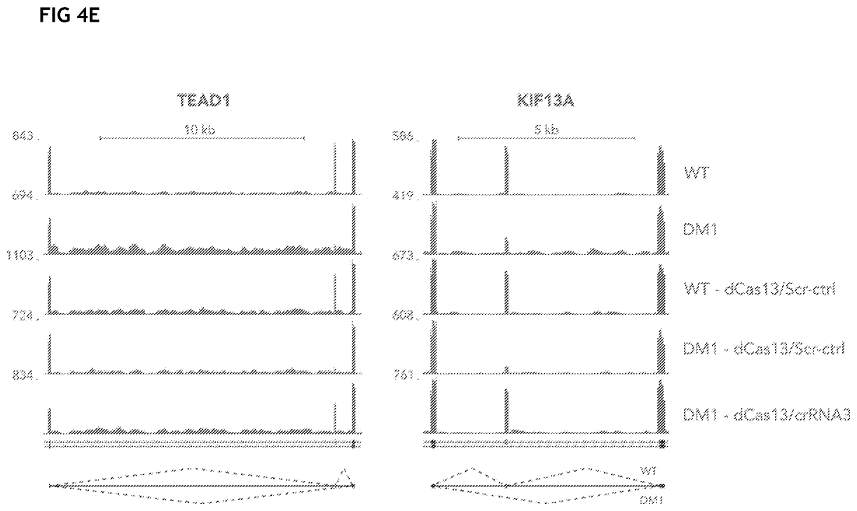

SEQ ID NOs: 7-11 shows the polynucleotide sequences of the crRNAs shown in FIG. 4B.

SEQ ID NOs: 12-69 show the sequences of the Cas13 proteins shown in Table 1.

SEQ ID NOs: 70 and 71 show nuclear localisation signals.

SEQ ID NO: 72 shows the Kozak sequence.

SEQ ID NOs 73-80 show the crRNA spacer sequences shown in Table 2.

SEQ ID NO: 81 shows the repeat motif of Unverricht-Lundborg disease shown in Table 3.

### DETAILED DESCRIPTION OF THE INVENTION

dCas13 Protein

The invention relates to a catalytically inactive Cas13 protein (dCas13). Hence, the dCas13 protein does not elicit cleavage of the target RNA sequence but retains the other biological activity of the dCas13 protein, such as binding affinity to RNA and blocking activity compared to the unmodified Cas13.

Whilst not wishing to be bound by theory, the dCas13 is recruited to a target site in an RNA molecule as determined by a CRISPR RNA (crRNA). This promotes steric blocking of the dCas13 and/or crRNA at the target site in the RNA molecule, in such a way that it does not induce target cleavage. For example, where the dCas13 is targeted to a repeat expanded sequence in an RNA molecule (e.g., DMPK mRNA), the dCas13 and/or crRNA physically masks the repeat expanded sequence, preventing it from interaction with proteins. As another example, where the dCas13 is targeted to an upstream start codon (e.g., a start codon of an uORF), the dCas13 and/or crRNA physically masks the uATG.

Cas13 proteins are known to comprise a single multi-domain effector (Class II) and target RNA only (Type VI), and Cas13 proteins can be determined according to computational methods known in the art. Table 1 lists examples of naturally-occurring Cas13 proteins and indicates for each Cas13 protein the mammalian cell compatibility and nuclease activity. The mammalian cell compatibility of a Cas13 protein measures whether the Cas13 protein can be expressed, be properly folded, and show functional biological activity in mammalian cells. For example, some Cas13 proteins, including LweCas13a and LbfCas13a, fail to show functional biological activity in mammalian cells, and so are considered to be incompatible with mammalian cells. Nuclease activity can serve as a proxy for binding affinity to its target RNA sequence, as RNA degradation cannot occur without interactions between the Cas13 protein and its target RNA sequence. Based on the phylogeny of Cas13, the CRISPR-Cas13 system can be classified into Cas13a (previously known as C2C2), Cas13b, Cas13c, Cas13d, Cas13X and Cas13Y, all of which require a crRNA for the specific recognition of target RNA sequences.

A dCas13 protein useful with the invention may be derived from a naturally-occurring or a modified Cas13 protein. Modifications to Cas13 proteins are explained further below.

A naturally-occurring Cas13 protein that has good mammalian cell compatibility and high binding affinity to its target RNA sequence is considered to exhibit good blocking efficiency, and so are particularly useful with the invention. For example, a Cas13 protein which exhibits nuclease activity in mammalian cells and binds to its target RNA sequence with high affinity, e.g., with a K_{D} value of ≤100 nM, ≤50 nM, ≤10 nM, ≤5 nM, ≤1 nM, ≤0.5 nM, or ≤0.1 nM is particularly useful with the invention. Binding affinity (K_{D}) can be analysed by any suitable means known in the art, for example, by ELISA or Surface Plasmon Resonance. The nuclease activity of a Cas13 protein may be determined in a fluorescent reporter assay, such as an ECFP/mIFP bidirectional reporter assay, carried out under physiological conditions in cell culture. A Cas13 protein which reduces expression of the fluorescent reporter by at least 25% relative to a negative control (e.g., cells not transfected with the Cas13 protein) is particularly useful with the invention.

A Cas13 protein useful with the invention may be a member of the Cas13b, Cas13a, Cas13d, Cas13X, Cas13Y, or Cas13bt family.

The Cas13 may be PspCas13b, LshCas13a, LwaCas13a, LbmCas13a, LbnCas13a, LbfCas13a, RcsCas13a, RcrCas13a, RcdCas13a, LbuCas13a, HheCas13a, LspCas13a, BzoCas13b, PinCas13b, PbuCas13b, PsmCas13b, RanCas13b, PauCas13b, Pin2Cas13b, PguCas13b, PgiCas13b, Pin3Cas13b, Cas13bt1, Cas13bt3, FnbCas13c, AspCas13c, UrCas13d, P1E0Cas13d, AdmCas13d, RfxCas13d, Cas13X.1, Cas13X.2, Cas13Y.1, Cas13Y.2, Cas13Y.3, Cas13Y.5, Cas13bt1, or Cas13bt3. These Cas13 proteins have good human cell compatibility and high binding affinity (e.g., see Table 1).

The Cas13 may be PspCas13b, LshCas13a, LwaCas13a, LbmCas13a, LbnCas13a, LbfCas13a, RcsCas13a, RcrCas13a, RcdCas13a, LbuCas13a, HheCas13a, LspCas13a, BzoCas13b, PinCas13b, PbuCas13b, PsmCas13b, RanCas13b, PauCas13b, Pin2Cas13b, PguCas13b, PgiCas13b, Pin3Cas13b, Cas13bt1, Cas13bt3, FnbCas13c, AspCas13c, UrCas13d, P1E0Cas13d, AdmCas13d, RfxCas13d, Cas13X.1, Cas13X.2, Cas13Y.1, Cas13Y.2, Cas13Y.3, or Cas13Y.5. These Cas13 proteins have good human cell compatibility and high binding affinity (e.g., see Table 1).

The Cas13 may be PspCas13b, PinCas13b, PguCas13b, PgiCas13b, Cas13X.1, Cas13X.2, Cas13Y.1, Cas13Y.2, or Cas13Y.3. These Cas13 proteins have good human cell compatibility and high binding affinity (e.g., see Table 1).

The Cas13 protein may be a member of the Cas13b family, i.e., a Cas13b protein. The Cas13b proteins are particularly useful with the invention because they have good human cell compatibility and high binding affinity (e.g., see Table 1), and so have good blocking efficiency. Furthermore, these proteins interact with shorter crRNAs to improve blocking efficiency and can be truncated to optimise vector packaging, as explained further below.

The Cas13 protein may be Psp-Cas13b (Accession No. WP_044065294).

The Cas13 protein may not be a Cas13a protein. For example, the Cas13 protein may not be LshCas13a (WP_018451595. 1). The Cas13 protein may not be LbuCas13a (WP_015770004.1). The Cas13 protein may not be LbaCas13a (see Reference 3).

The Cas13 protein may not be a Cas13d protein. For example, the Cas13 protein may not be CasRxCas13d (see Reference 3).

The naturally-occurring Cas13 protein may be modified to inactivate its ribonuclease activity by mutating one or more amino acid residues in the RxxxxH motif (wherein x is any amino acid) of both of the two HEPN RNase domains responsible for ribonuclease activity, thereby eliminating RNA cleavage without affecting crRNA array processing or target RNA binding. Hence, the dCas13 protein may comprise mutation of one or more amino acid residues (e.g., 1, 2, 3, 4, 5 or 6 amino acid resides) in the RxxxxH motif of the HEPN-1 domain relative to the wild-type and in the RxxxxH motif of the HEPN-2 domain relative to the wild-type. The mutation may be at any of the residues in the RxxxxH motif of the HEPN-1 domain and in the RxxxxH motif of the HEPN-2 domain. The mutation may be substitution, replacement or deletion. For example, the first R residue or the last H residue in a RxxxxH motif may be substituted with a different amino acid, such as an alanine (A). The substitution is typically with a non-conservative amino acid.

For example, PspCas13b may be modified to inactivate its ribonuclease activity by substituting the histidine residues at positions 133 and 1058, which correspond to the histidine residues of the two RxxxxH motifs, with alanine residues to form Psp-dCas13b (SEQ ID NO: 1).

A dCas13 useful with the invention may comprise or consist of an amino acid sequence having a sequence identity of ≥85%, ≥90%, ≥95%, ≥96%, ≥97%, ≥98%, ≥99%, or 100% to SEQ ID NO: 1, provided that: (a) one or more of the amino acid residues corresponding to the amino acid residues at positions 128 to 133 of SEQ ID NO: 1 are mutated; and (b) one or more of the amino acid residues corresponding to the amino acid residues at positions 1053 to 1058 of SEQ ID NO: 1 are mutated. A dCas13 useful with the invention may comprise or consist of an amino acid sequence having a sequence identity of ≥85%, ≥90%, ≥95%, ≥96%, ≥97%, ≥98%, ≥99%, or 100% to SEQ ID NO: 1, provided that the amino acid residues corresponding to the amino acid residues at positions 133 and 1058 of SEQ ID NO: 1 are mutated. A dCas13 useful with the invention may comprise or consist of an amino acid sequence having a sequence identity of ≥85%, ≥90%, ≥95%, ≥96%, ≥97%, ≥98%, ≥99%, or 100% to SEQ ID NO: 1, provided that the amino acid residues corresponding to the amino acid residues at positions 133 and 1058 of SEQ ID NO: 1 are alanine. A dCas13 useful with the invention may consist of SEQ ID NO: 1.

Methods to determine nuclease activity, binding affinity to its target RNA sequence and blocking activity of a Cas13 or a dCas13 protein are known in the art, and described in the examples herein, e.g., fluorescent reporter assay, endogenous RNA blocking assay, or electrophoretic mobility shift assay.

The dCas13 may have a size of about 700 to about 1200 amino acids.

The naturally-occurring Cas13 protein may be modified to reduce its size whilst retaining its blocking activity. Such smaller dCas13 proteins are advantageous because the size of the nucleic acid sequence encoding the dCas13 protein would be minimised for packaging in vectors for cell delivery, particularly in vectors with limited transgene packaging capacity. For example, AAV (packaging capacity about 4.7 kb) has limited capacity to include the nucleic acid sequence encoding the full-length dCas13 protein (about 3 kb in length) in addition to other necessary elements in an expression cassette.

The size of a dCas13 protein may be reduced by C-terminal truncation, such as truncation of the HEPN-2 domain which resides in the C-terminus region of a dCas13 protein. Hence, the invention provides a dCas13 protein comprising truncation in the C-terminus compared to the unmodified protein. The dCas13 protein may be truncated by ≤50, ≤100, ≤150, ≤200, ≤250, or ≤300 amino acid residues from the C-terminus compared to the unmodified protein. For example, the dCas13 protein may have a size of ≤1050, ≤1000, ≤950, ≤900, ≤850, ≤800, ≤750, or ≤700 amino acids.

The truncation does not substantially alter the steric-blocking efficiency of the dCas13 protein. Hence, the truncated dCas13 protein retains its binding affinity to the target RNA sequence and its blocking activity compared to the unmodified dCas13. The truncated dCas13 protein may be used with the methods and uses described herein.

The dCas13 protein may lack the entire HEPN-2 domain. The dCas13 protein may substantially lack the HEPN-2 domain. The location and length of the HEPN-2 domain in a given Cas13 protein or orthologue may be determined using several bioinformatics tools known in the art (e.g., ThreaDom) prior to truncation. The HEPN-2 domain of a dCas13 protein may be dispensable with minimal effect on its activity, such as blocking efficiency. In particular, the inventors have shown that the truncation of the C-terminus domain which contains the HEPN-2 domain of a Psp-dCas13b did not lead to decreased blocking efficiency. It has also been shown that establishment and maintenance of interactions between the crRNA, dCas13b, and target RNA sequence does not require an intact HEPN-2 domain. Considering the high degree of sequence similarity between members of the Cas13b family, truncation of the HEPN-2 domain in any of the Cas13b members may result in a modified protein that has minimal effect on its steric blocking efficiency. Hence, the Cas13 protein of the truncated dCas13 protein may be a member of the Cas13b family. The Cas13 protein may be Psp-Cas13b, Bzo-Cas13b, Pin-Cas13b, Pbu-Cas13b, Asp-Cas13b, Psm-Cas13b, Ran-Cas13b, Pau-Cas13b, Psa-Cas13b, Pin2-Cas13b, Cca-Cas13b, Pgu-Cas13b, Fbr-Cas13b, Pgi-Cas13b or Pin3-Cas13b. The Cas13 protein may be Psp-Cas13b.

The invention also provides a dCas13 protein comprising or consisting of an amino acid sequence having a sequence identity of ≥85%, ≥90%, ≥95%, ≥96%, ≥97%, ≥98%, ≥99%, or 100% to SEQ ID NO: 2 or 3, provided that: (a) one or more of the amino acid residues corresponding to the amino acid residues at positions 128 to 132 are mutated; and (b) the amino acid residue at position 133 is histidine. The invention also provides a dCas13 protein comprising or consisting of an amino acid sequence having a sequence identity of ≥85%, ≥90%, ≥95%, ≥96%, ≥97%, ≥98%, ≥99%, or 100% to SEQ ID NO: 2 or 3, provided that the amino acid residue corresponding to the amino acid residue at position 133 of SEQ ID NO: 2 or 3 is alanine or is not histidine. The dCas13 protein may consist of SEQ ID NO: 2. The dCas13 protein may consist of SEQ ID NO: 3. The dCas13 protein may be used with the methods and uses described herein.

The dCas13 protein useful with the invention may contain modifications relative to any of SEQ ID NOs: 1 to 3, such as amino acid substitutions, additions or deletions, provided that: (a) one or more of the amino acid residues corresponding to the amino acid residues at positions 128 to 133 of SEQ ID NO: 1 are mutated and one or more of the amino acid residues corresponding to the amino acid residues at positions 1054 to 1058 of SEQ ID NO: 1 are mutated; (b) the amino acid residues corresponding to the amino acid residues at positions 133 and 1058 of SEQ ID NO: 1 are alanine; (c) one or more of the amino acid residues corresponding to the amino acid residues at positions 128 to 132 are mutated and the amino acid residue at position 133 is histidine; or (d) the amino acid residue corresponding to the amino acid residue at position 133 of SEQ ID NO: 2 or 3 is alanine or is not histidine. For example, the dCas13 protein may comprise 1, 2, 3, 4, 5, 6, 7, 8, 9, 10 or more amino acid residues that are substituted, deleted or added, in any combination.

The dCas13 protein may comprise further mutations. For example, the dCas13 protein may have additions, deletions or substitutions of amino acid residues which do not substantially alter the steric-blocking efficiency of the dCas13 protein. Those individual sites or regions of the Cas13 protein, which can be altered without affecting steric-blocking efficiency can be determined by examination of the structure of the dCas13 domains, for example. Alternatively, the regions which would tolerate amino acid substitutions may be determined by alanine scanning mutagenesis (4). In this method, selected amino acid residues are individually substituted with a neutral amino acid (e.g., alanine) in order to determine the effects on steric-blocking efficiency.

The dCas13 protein may contain conservative amino acid changes which are least likely to perturb the structure and/or function of the protein. For example, the variant may comprise one or more conservative amino acid changes within any of SEQ ID NOs: 1 to 3. Conservative amino acid changes generally involve substitution of one amino acid with another that is similar in structure and/or function (e.g., amino acids with side chains similar in size, charge and shape). Amino acid residues having similar side chains are known in the art. These include amino acids with basic side, chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residue within the dCas13 protein can be replaced with other amino acid residues having similar side chains and the altered protein can be tested for retained function using the functional assays described herein. Modifications can be introduced by standard techniques known in the art, such as site-specific mutagenesis (5) and PCR-mediated mutagenesis, provided that activity, e.g., the ability to bind to the target RNA sequence and hence steric blocking, is retained.

The dCas13 protein may be codon optimized to increase expression levels of the respective protein in host cells as compared to the unaltered sequence. Methods for codon optimisation are known in the art, e.g., GeneScript OptimumGene™ algorithm can be used.

It is within the teaching herein that the dCas13 protein may be fused to a peptide for purification or detection. The peptide may be an affinity tag, such as a HA-tag which correspond to amino acids 98 to 106 of human influenza hemagglutinin, polyhistidine (His) (H₆; SEQ ID NO: 82), c-myc and/or FLAG. The peptide may be a reporter protein, such as a fluorescent reporter. The peptide may be fused to the N- or C-terminal of the dCas13 protein, for example.

The dCas13 protein may be fused to a localisation signal peptide, e.g., for transportation to a particular location in a cell. The signal peptide may be a nuclear localisation signal (NLS) peptide. The nuclear localisation signals peptide may be fused to or positioned in proximity (e.g., within 5 amino acids) to the N- and/or C-terminus of the dCas13. For example, the nuclear localisation signals may be fused to the N-terminus of the dCas13 protein for optimal expression and cytoplasmic targeting in eukaryotic cells, such as human cells. Exemplary nuclear localisation signals are the nuclear localisation signal of SV40 large T antigen (PKKKRRV; SEQ ID NO: 70) and the nuclear localisation signal of nucleoplasmin (KRPAATKKAGQAKKKK; SEQ ID NO: 71). Other nuclear localisation signals are known in the art, e.g., see References 6,7,8.

The invention also refers to a polynucleotide encoding a dCas13 protein described herein.

In one embodiment, the dCas13 protein may not be fused to a biologically active entity, such as a peptide comprising an ADAR deaminase domain.

### CRISPR RNA

A CRISPR RNA (crRNA) useful with the invention comprises a dCas13-specific direct repeat and a spacer which is capable of specifically hybridizing with the target RNA sequence. The dCas13-specific direct repeat selectively binds with sufficient affinity to a dCas13 protein described herein and recruits it to the target site in an RNA molecule as determined by the spacer. This promotes steric blocking of the dCas13 and/or crRNA at the target RNA sequence.

The dCas13-specific direct repeats useful with the invention is dependent on the specific dCas13 protein. For instance, Cas13b from different species can have different direct repeat sequences and/or secondary structures. The dCas13-specific direct repeats in the crRNA provided herein can be chosen based on the specific dCas13 used. Direct repeat sequences functioning together with Cas13 proteins of various bacterial species may be identified by bioinformatic analysis of sequence repeats occurring in the respective CRISPR/Cas operons and by experimental binding studies of Cas13 protein together with putative direct repeat sequence flanked target sequences.

The dCas13-specific direct repeat may be about 30 to about 90 (e.g., about 30, 40, 50, 60, 70, 80, or 90) nucleotides in length.

The crRNA may comprise more than one (e.g., at least two, three, four, five, six, or seven) direct repeats. The two or more direct repeats may have the same or different length.

The direct repeat may form a hairpin structure capable of interacting with the Cas13 protein to form a complex.

The dCas13-specific direct repeat in the crRNA described herein may be a Cas13b-specific direct repeat.

The spacer can be designed to target any sequence in a target RNA. The spacer is designed to complement the target RNA sequence. The RNA-targeting sequence in the crRNA may fully complement, substantially complement or partially complement the target RNA sequence.

The crRNA may comprise more than one (e.g., at least two, three, four, five, six, or seven) spacers. The spacers can bind to the same or different target sequences in the same target RNA or can bind to different target RNAs. The two or more spacers can have the same or different length.

The spacer may have a length of between 9 to 45 (e.g., 9 to 15, 15 to 30, 18 to 24, 25 to 40, 25 to 35, 25 to 30) nucleotides.

The spacer having a length of between 18 to 24 (e.g., 18, 19, 20, 21, 22, 23 or 24) nucleotides is particularly advantageous because it may increase the steric blocking efficiency of the dCas13 protein, e.g., dCas13b protein, with which the crRNA interacts. In particular, the inventor found that when the length of the spacer sequence of the crRNA specific for PspCas13b was reduced from the native length of about 30 nucleotides to between 18 to 24 nucleotides, the steric blocking efficiency was increased (see Examples).

The improved blocking efficiency may be attributed to improved binding affinity of the dCas13 to the target RNA sequence. Without wishing to be bound by theory, it is considered that crRNA:Cas13b hybridisation requires that the HEPN1 and Helical-2 domains are open (e.g., see Reference 9). Upon hybridisation of the crRNA with the target RNA, both the HEPN1 and Helical-2 domains return to their closed state in order to stabilise the crRNA:target RNA interaction. A reduced base-pairing between the crRNA and its target RNA sequence enables more efficient closing of the HEPN1 and Helical-2 domains, thus mediating more sterically favourable binding. In contrast, increased base-pairing between crRNA and its target RNA may sterically block the closing of the HEPN1 and Helical-2 domains, leading to an open conformation, which is known to be less stable. Indeed, a recent structural study showed that crRNA hybridises with Cas13b proteins by accessing the central channel between the HEPN1 and Helical-2 domains. All members of the Cas13b family are amenable to the short-crRNA effect, due to their need for conformational rearrangement to establish strong binding to target RNA sequences.

Hence, the invention provides a crRNA specific for dCas13b comprises: (i) a dCas13b-specific direct repeat, and (ii) a spacer which is capable of specifically hybridizing with the target RNA sequence and having length of between 18 to 24 nucleotides. This crRNA is specifically useful with the methods and uses of the invention.

The crRNA sequence may comprise conservative mutations that do not change the length and extend of the hairpin loop in the direct-repeat region and that preserve the function and activity of the crRNA.

The crRNA may comprise or consist of any of SEQ ID NOs: 4 or 7 to 11.

The crRNA may be a modified crRNA, i.e., it comprises at least one modified nucleoside (e.g., at least one modified sugar moiety and/or at least one modified nucleobase moiety) and/or at least one modified internucleoside linkage. The crRNA may be modified such that the stability of the modified crRNA in human cells is improved relative to the unmodified crRNA. For example, the crRNA may be modified by 3′-end capping with inverted thymidine, addition of 2′-O-methylation, and/or addition of phosphorothioate linkage at the 3′-end.

### Regulatory Element and Target RNA

The invention relates to any regulatory element in a target RNA, where the regulatory element regulates the expression or activity of a gene of interest. The regulatory element may regulate when, where and how much the gene is expressed in the form of RNA or protein, and/or its activity.

The regulatory element is in a target RNA. The target RNA may be any RNA molecules endogenous or exogenous to a eukaryotic cell, and can be protein-coding or non-protein-coding. For example, a target RNA can be messenger RNA (mRNA), ribosomal RNA (rRNA), signal recognition particle RNA (SRP RNA), transfer RNA (tRNA), small nuclear RNA (snRNA), small nucleolar RNA (snoRNA), antisense RNA (aRNA), long noncoding RNA (lncRNA), pseudogene, circular RNA (circRNA), long intergenic non-coding RNA (lincRNA), microRNA (miRNA), piwi-interacting RNA (piRNA), small interfering RNA (siRNA), short hairpin RNA (shRNA), retrotransposon RNA, viral genome RNA, or viral noncoding RNA.

The target RNA is typically a mature messenger RNA (mRNA), but may also be a precursor mRNA (pre-mRNA). The pre-mRNA may have undergone partial splicing, i.e., a partially processed mRNA transcript.

For example, the target RNA is a repeat-expanded mRNA, such as CSTB, XYLT1, GLS, PPP2R2B, AFF2, CBL2, AFF3, DIP2B, FMR1, FMR1, FMR1, NOTCH2NLC, LRP12, GIPC1, LOC642361 and NUTM2B-AS1b, RFC1, ATXN10, TAF1, CNBP, ZNF713, TCF4, FXN, NOP56, C9orf72, BEAN1/TK2b, SAMD12, STARD7, Mar-06, YEATS2, TNRC6A, RAPGEF2, DAB1, ATN1, HTT, AR, ATXN1, ATXN2, ATXN3, CACNA1A, ATXN7, TBP, COMP, FOXL2, RUNX2, PHOX2B, HOXA13, ZIC2, PABPN1, HOXD13, SOX3, ARX, ATXN8OS and ATXN8b, DMPK, or JPH3 mRNA.

The target RNA may be DMPK, TCF4, CNBP, JPH3, C9orf72, FMR1, FMR1, NOP56, or BEAN1/TK2b mRNA.

The regulatory element may be a cis-regulatory element or a trans-regulatory element. The regulatory element may be a microsatellite repeat sequence, a splicing factor binding site, a snRNP binding site, an exonic splicing enhancer, an exonic splicing silencer, a protein binding site, an internal ribosome entry site (IRES), a hairpin structure, a stem-loop structure, a pseudoknot, a start codon of an open reading frame, a start codon of an upstream open reading frame (uORF), a Kozak sequence, a miRNA binding site, a siRNA binding site, or a piRNA binding site.

For example, the expanded repeat sequence may be a repeat sequence shown in Table 2 or 3. The expanded repeat sequence may be (CTG)n in the 3′-UTR DMPK mRNA, (CTG)n in the intron of TCF4 mRNA, (CCTG)n in the intron of CNBP mRNA, (CTG)n in the 3′-UTR of JPH2 mRNA, (GGGGCC)n in the intron of C9orf72 mRNA, (CGG)n in 5′-UTR of FMR1, (GGCCTG)n in the intron of NOP56, (TGGAA/TTCCA)n in the intron of BEAN1/TK2b or (ATTCT)n in the intron of ATXN10.

For example, the protein binding site may be a HuR binding site with the consensus motif 5‘-NNUUNNUUU-’3.

For example, the snRNP binding site may be a U2 snRNP binding site. The U2 snRNP binding site may comprise the 3′ splice site consensus sequence CAG|G and/or the 5′ splice site consensus sequence MAG|GTRAGT in pre-mRNA.

For example, the Kozak sequence may be the sequence 5′-(gcc)gccRccAUGG-3′ (SEQ ID NO: 72).

Hence, the spacer of the crRNA may comprise or consist of the sequence: SEQ ID NO: 73, 74, or 76, as shown in Table 2. See, for example, the spacer of the crRNA set out in SEQ ID NOs: 4 and 7 to 11.

### Polynucleotide, Vector and Host Cell

The invention also relates to a polynucleotide comprising a sequence encoding a dCas13 protein and/or a crRNA described herein. The polynucleotide may comprise a sequence encoding a dCas13 protein and a crRNA described herein. Polynucleotides which encode the dCas13 protein and/or crRNA can be obtained by methods well known to those skilled in the art.

A polynucleotide of the invention may be provided in the form of an expression cassette, which includes control sequences operably linked to the inserted sequence, thus allowing for expression of the dCas13 protein or crRNA described herein in vivo. Hence, the invention provides an expression cassette comprising a polynucleotide encoding the crRNA and/or dCas13 protein of the invention.

For example, the sequence encoding the dCas13 protein or crRNA may be operably linked to a promoter. Appropriate promoters are known in the art and described herein, e.g., a polymerase III promoter, such as a polymerase-3 U6 (U6:3) promoter. The sequence encoding dCas13 may be operably linked to a nuclear localization signal, e.g., a nuclear localization signal described herein. The sequence encoding a dCas13 protein or the crRNA may be further operably linked to a sequence that encodes one or more reporter genes. Appropriate reporter genes are well known in the art, e.g., fluorescent reporters.

These expression cassettes, in turn, are typically provided within vectors. Hence, the invention provides a vector encoding a polypeptide or expression cassette described herein. The vector may be a vector for cloning purposes (e.g., a plasmid). The vector may be a vector for expression of the polynucleotide in a cell. The vector may be a viral vector, such as an adeno-associated viral vector (AAV), e.g., AAV9, or a lentiviral vector. The vector may comprise any virus that targets the dCas13 protein and the crRNA to a specific cell type. The polynucleotide encoding the dCas13 protein and crRNA may be packaged into one or more vectors (e.g., plasmid or viral vectors). General methods by which the vectors may be constructed, transfection methods and culture methods are well known to those skilled in the art (e.g, see Reference 10).

The dCas13 protein and crRNA described herein are delivered to a cell. The dCas13 protein may be delivered in the form of a protein or a polynucleotide, such as in expression cassette or a vector. The crRNA is delivered in the form of a polynucleotide.

Hence, the invention also provides a host cell comprising a dCas13 protein, crRNA, polynucleotide, expression cassette or vector described herein. The dCas13 protein, a crRNA, polynucleotide, expression cassette or vector may be introduced transiently or permanently into the host cell, allowing expression of an oligonucleotide or conjugated oligonucleotide from the expression cassette or vector. The cell may be a eukaryotic cell, such as a mammalian cell (e.g., a rodent cell, a human cell, a non-human primate cell). Suitable cells include naturally-occurring cells; genetically modified cells (e.g., cells genetically modified in a laboratory); and cells manipulated in vitro in any way. In some cases, the cell is isolated.

### Composition

The invention provides a composition comprising a dCas13 protein, crRNA, polynucleotide, expression cassette or vector described herein. The composition may comprise a combination of one or more of the crRNAs of the invention. Each crRNA may be targeted to a different (but possibly overlapping) sequence in the same target RNA. Alternatively, each crRNA may be targeted to a different target RNA.

The composition may be a pharmaceutical composition. The pharmaceutical composition may further comprise a carrier (e.g., water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other materials well known to those skilled in the art. Such materials are typically non-toxic and does not interfere with the efficacy of the active ingredient. The precise nature of the carrier or other material may be determined by the skilled person according to the route of administration.

The pharmaceutical composition may further comprise one or more pharmaceutically acceptable salts (e.g., a mineral acid salt such as a hydrochloride, a hydrobromide, a phosphate, a sulphate, etc.) and the salts of organic acids (e.g., acetates, propionates, malonates, benzoates, etc.).

The pharmaceutical composition may comprise a delivery system, such as liposomes and emulsions. In certain embodiments, organic solvents such as dimethyl sulfoxide are used.

The pharmaceutical composition may comprise one or more tissue-specific delivery molecules designed to deliver a dCas13 protein, crRNA, polynucleotide, expression cassette or vector described herein to specific tissues or cell types. For example, the delivery molecule may comprise liposomes coated with a tissue-specific antibody.

For delayed release, a vector may be included in a pharmaceutical composition which is formulated for slow release, such as in microcapsules formed from biocompatible polymers or in liposomal carrier systems according to methods known in the art.

Pharmaceutical compositions of the invention may comprise additional active agents, for example a drug or a pro-drug.

The pharmaceutical composition may be formulated to be administered by any administration route, e.g., as described herein. The pharmaceutical composition is typically administered by injection. In such embodiments, the pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives).

### Method and Use

The invention also relates to the methods and uses of a dCas13 protein, crRNA, polynucleotide, expression cassette, vector or host cell described herein. The methods and uses of the invention may be non-therapeutic or therapeutic, as explained further below.

The methods and uses of the invention may comprise modulating the function of a regulatory element in a target RNA. In particular, the dCas13 protein, crRNA, polynucleotide, expression cassette, vector or host cell are used in a method of modulating the function of a regulatory element in a target RNA.

The methods and uses of the invention may be in vitro, ex vivo or in vivo. For example, the invention also provides an in vitro method of modulating the function of a regulatory element in a target RNA, comprising delivering to a cell a catalytically inactive Cas13 protein (dCas13) and a CRISPR RNA (crRNA), wherein the crRNA recruits the dCas13 protein to the regulatory element, such that the dCas13 protein sterically blocks the regulatory element, optionally wherein the Cas13 protein is a member of the Cas13b family, Cas13X family, Cas13Y family or Cas13bt family.

Hence, in some embodiments, the method or use of the invention is not a treatment of the human or animal body by surgery or therapy and is not a diagnostic method practised on the human or animal body.

The methods and uses of the invention may comprise modulating (e.g., increasing, decreasing, or restoring) the availability, expression and/or activity of a nucleic acid or protein of interest by modulating the function of a regulatory element in a target RNA, wherein the target RNA encodes or regulates the nucleic acid or protein of interest.

The regulatory element may be an expanded repeat sequence, e.g., in a target RNA which causes expansion repeat diseases, e.g., DM1. In such embodiments, modulating of the function of the regulatory element may result in increasing, decreasing or restoring the availability, expression and/or activity of a protein regulated by the target RNA.

The regulatory element may be a splicing element, a start codon of an open reading frame, or a start codon of an upstream open reading frame (uORF). Hence, the invention also relates to blocking ribosomal attachment or translation, comprising modulating the function of a regulatory element in a nucleic acid according to the method of the invention, or modulating the availability, expression and/or activity of a nucleic acid or protein of interest according to the method of the invention, wherein the crRNA comprises a spacer that is complementary to a start codon of an open reading frame or a start codon of an upstream open reading frame (uORF). In such embodiments, modulating of the function of the regulatory element may result in increasing, decreasing or restoring the expression and/or activity of a protein encoded by the target RNA.

The regulatory element may be a splicing element. Hence, the invention also relates to inducing splice switching, comprising modulating the function of a regulatory element in a nucleic acid according to the method of the invention, or modulating the availability, expression and/or activity of a nucleic acid or protein of interest according to the method of the invention, wherein the crRNA comprises a spacer that is complementary to a splicing element, such as an RNP-binding site.

The regulatory element may be a nucleic acid (e.g., miRNA) binding site. In such embodiments, modulating the function of the regulatory element may result in increasing, decreasing or restoring the expression and/or activity of a nucleic acid and/or protein regulated by the target RNA. Hence, the invention also relates to blocking a miRNA-binding site, comprising modulating the function of a regulatory element in a nucleic acid according to the method of the invention, or modulating the availability, expression and/or activity of a nucleic acid or protein of interest according to the method of the invention, wherein the crRNA comprises a spacer that is complementary to a miRNA-binding site.

In the embodiments of the invention where the availability of a nucleic acid or protein is decreased, the availability, e.g., as determined by the unbound level of the nucleic acid or protein, may be decreased by ≥50% (i.e., 50% or more), ≥60%, ≥70%, ≥80%, ≥90% or 100% compared to the availability of the nucleic acid or protein in cells which have not been in contact with a dCas13 protein or crRNA described herein.

In the embodiments of the invention where the availability of a nucleic acid or protein is restored, the availability, e.g., as determined by the unbound level of the nucleic acid or protein, may be restored by ≥50% (i.e., 50% or more), ≥60%, ≥70%, ≥80%, ≥90% or 100% compared to the availability of the nucleic acid or protein in cells which have not been in contact with a dCas13 protein or crRNA described herein.

In the embodiments of the invention where the availability of a nucleic acid or protein is increased, the availability, e.g., as determined by the unbound level of the nucleic acid or protein, may be increased by ≥50% (i.e., 50% or more), ≥60%, ≥70%, ≥80%, ≥90% or 100% compared to the availability of the nucleic acid or protein in cells which have not been in contact with a dCas13 protein or crRNA described herein.

The invention also relates to increasing, decreasing or restoring the expression and/or activity of a nucleic acid or protein, comprising a method of modulating the function of a regulatory element in a target RNA as described herein.

In the embodiments of the invention where expression and/or activity of a nucleic acid or protein is increased, the expression and/or activity may be increased by ≥50% (i.e., 50% or more), ≥60%, ≥70%, ≥80%, ≥90%, ≥100% or ≥200% compared to the expression and/or activity in cells which have not been in contact with a dCas13 protein or crRNA described herein.

In the embodiments of the invention where expression and/or activity of a nucleic acid or protein is reduced, the expression and/or activity may be reduced by ≥50% (i.e., 50% or more), ≥60%, ≥70%, ≥80%, ≥90% or 100% compared to the expression and/or activity in cells which have not been in contact with a dCas13 protein or crRNA described herein.

In the embodiments of the invention where expression and/or activity of a nucleic acid or protein is restored, the expression and/or activity may be restored by ≥15% (i.e., 15% or more), ≥20%, ≥30%, 40%, ≥50%, ≥60%, ≥70%, ≥80%, ≥90% or 100% compared to the expression and/or activity in cells which have not been in contact with a dCas13 protein or crRNA described herein.

The methods and uses of the invention may include a step of determining: (i) the expression and/or activity level of the target RNA, (ii) the expression and/or activity level of the protein encoded or regulated by the target RNA, and/or (iii) the amount and/or activity level of a protein or nucleic acid that would have been bound to the target RNA; in a sample from a subject. Methods of determining the expression and/or activity levels of nucleic acids and proteins are known in the art. For example, RNA from a sample may be isolated and tested by hybridisation or PCR techniques as known in the art. Alternatively, protein expression assays can be performed in vivo, in situ, i.e., directly upon tissue sections (fixed and/or frozen) of patient tissue obtained from biopsies or resections, such that no nucleic acid purification is necessary. Immunoassays may also be used, e.g., Western Blot or ELISA.

The invention also relates to a dCas13 protein, crRNA, polynucleotide, expression cassette, vector or host cell described herein for use in a method of therapy practiced on the human or animal body. The invention further relates to the use of a dCas13 protein, crRNA, polynucleotide, expression cassette, vector or host cell described herein in the manufacture of a medicament for a method for treatment. The invention also relates to the use of a dCas13 protein, crRNA, polynucleotide, expression cassette, vector or host cell described herein for a method for treatment. The invention relates to a method of treating or preventing a disease comprising administering to the subject a therapeutically effective amount of a dCas13 protein, crRNA, polynucleotide, expression cassette, vector or host cell described herein.

The methods and uses of the invention may comprise inhibiting the disease state, e.g., arresting its development; and/or relieving the disease state, e.g., causing regression of the disease state until a desired endpoint is reached. The methods and uses of the invention may comprise the amelioration or the reduction of the severity, duration or frequency of a symptom of the disease state (e.g., lessen the pain or discomfort), and such amelioration may or may not be directly affecting the disease.

The methods and uses of the invention relate to delivering a dCas13 protein, crRNA, polynucleotide, expression cassette, vector or host cell described herein to a cell.

The cell may be a eukaryotic cell (e.g., a human cell). The cell may be from non-human animals such as mice, rats, rabbits, sheep, pigs, cows, cats, or dogs is also contemplated.

In particular, the methods and uses of the invention may involve delivering to a cell a vector (e.g., a viral vector such as AAV9) comprising an expression cassette comprising a polynucleotide encoding a dCas13 protein (e.g., dCas13b protein) and a crRNA comprising dCas13b-specific direct repeats. The dCas13b protein may be Psp-dCas13b, e.g., SEQ ID NO: 1, 2, or 3

The delivery may be either via a single dose or multiple doses.

Typically, the invention relates to methods and uses for a human subject in need thereof. However, non-human animal subjects, such as mice, rats, rabbits, sheep, pigs, cows, cats, or dogs, is also contemplated.

The invention relates to analysing samples from subjects. The sample may be tissues, cells and biological fluids isolated from a subject, as well as tissues, cells and fluids present within a subject. The sample may be blood and a fraction or component of blood including blood serum, blood plasma, or lymph.

The detection assays may be performed in situ, in which case the sample is a tissue section (fixed and/or frozen) of the tissue obtained from biopsies or resections from a subject.

The dCas13 protein, crRNA, polynucleotide, expression cassette, vector or host cell described herein may be administered subcutaneously, intravenously, intradermally, orally, intranasally, intramuscularly, intracranially, intrathecally, intracerebroventricularly, intravitreally, or topically (e.g., in the form of a cream for skin).

Dosages and dosage regimes appropriate for use with the invention can be determined within the normal skill of the medical practitioner responsible for administration of the composition. For example, for treatment purposes, a therapeutically effective amount of the dCas13 protein, crRNA, polynucleotide, expression cassette, vector or host cell described herein would be administered to such a subject. A therapeutically effective amount is an amount which is effective to ameliorate one or more symptoms of the disorder.

The person skilled in the art understands that dosage may be determined according to various parameters, especially according to the age, weight and condition of the patient to be treated; the vector choice, the target cell, organism, or tissue, the degree of transformation/modification sought, the administration route, the administration mode, the type of transformation/modification sought, etc. A physician will be able to determine the required route of administration and dosage for any particular patient.

For example, a polynucleotide encoding a dCas13 protein and a crRNA described herein may be administered at a dose of between about 10 microgram/kg and about 300 milligram/kg bodyweight, such as about 50 mg/kg, by intramuscular injection or intravascular injection. In embodiments where the polynucleotide encoding a dCas13 protein and a crRNA described herein is delivered in a viral vector (e.g., AAV9), the viral vector may be administered at a dose between about 1×10⁸ to about 1×10¹⁵ vector genomes (vg) per kilogram of body weight. The dosage may be adjusted to balance the therapeutic benefit against any side effects. Other effective dosages can be readily established by one of ordinary skill in the art through routine trials establishing dose response curves.

RNA delivery is a useful method of in vivo delivery. An RNA encoding dCas13 and crRNA described herein may be delivered into cells using liposomes, nanoparticles, microvesicles, or exosomes. For example, dCas13 mRNA and crRNA can be packaged into liposomal particles for delivery in vivo. Liposomal transection reagents such as lipofectamine from Life Technologies and other reagents on the market can effectively deliver RNA molecules into the liver.

The methods and uses of the invention may relate to treating or preventing repeat expansion diseases (e.g., type I myotonic dystrophy (DM1)), diseases associated with miRNA dysregulation, diseases associated with dysregulated translation, or diseases associated with abnormal splicing (e.g., spinal muscular atrophy (SMA) and Duchenne muscular dystrophy (DMD)). These diseases are explained further below.

### Repeat Expansion Diseases

The methods and uses of the invention may relate to treating or preventing repeat expansion diseases. The methods and uses of the invention are particularly useful in treating or preventing repeat expansion diseases where repeat expansion occurs in the non-coding region; and where somatic instability/RBP sequestration/RAN translation plays some role in disease pathology. The inventors have demonstrated that Psp-dCas13b can be programmed using crRNA to target, and sterically block, the expanded CTG repeat at the post-transcriptional level. This leads to de-sequestration of RBPs involved in splicing and subsequent reversal of the spliceopathy observed in DM1 patient-derived cells and in the cells of a DM1 mouse model. Based on the mechanism of action of Psp-dCas13b and the known DM1 disease mechanisms, the observed therapeutic effects would apply to other repeat expansion diseases, such as those where repeat expansion occurs in the non-coding region; and where somatic instability/RBP sequestration/RAN translation plays some role in disease pathology. Examples of such repeat expansion diseases are provided in Tables 2 and 3.

Hence, the invention provides a method of treating or preventing a disease listed in Table 2 or 3, comprising administering to the subject a therapeutically effective amount of the dCas13 protein and crRNA described herein. The invention also provides a dCas13 protein described herein for use in a method of treating or preventing a disease listed in Table 2 or 3, wherein the method comprises administering to the subject a therapeutically effective amount of the dCas13 protein and a crRNA. The invention also provides a crRNA described herein for use in a method of treating or preventing a disease listed in Table 2 or 3, wherein the method comprises administering to the subject a therapeutically effective amount of a dCas13 protein and the crRNA. Similarly, the invention also provides the use of a dCas13 protein described herein in the preparation of a medicament for a method of treating or preventing a disease listed in Table 2 or 3, wherein the method comprises administering to the subject a therapeutically effective amount of the dCas13 protein and a crRNA. The invention also provides the use of a crRNA described herein in the preparation of a medicament for a method of treating or preventing a disease listed in Table 2 or 3, wherein the method comprises administering to the subject a therapeutically effective amount of a dCas13 protein and the crRNA. The method may comprise modulating the function of a regulatory element in a nucleic acid according to the method of the invention, or modulating the availability, expression and/or activity of a nucleic acid or protein of interest according to the method of the invention. The crRNA may comprise a spacer that is complementary to a repeat expanded sequence in a target RNA of a gene listed in Table 2 or 3 associated with the disease to be treated or prevented.

The repeat expanded disease may be Unverricht-Lundborg disease, Baratela-Scott syndrome, Glutaminase deficiency, Spinocerebellar ataxia type 12, Fragile XE syndrome, Jacobsen syndrome, Intellectual disability associated with fragile site FRA2A, Intellectual disability associated with fragile site FRA12A, Fragile X syndrome, Fragile X-associated primary ovarian insufficiency, Fragile X-associated tremor/ataxia syndrome, Neuronal intranuclear inclusion disease, Oculopharyngodistal myopathy 1, Oculopharyngodistal myopathy 2, Oculopharyngeal myopathy with leukoencephalopathy, Cerebellar ataxia, neuropathy and vestibular areflexia syndrome, Spinocerebellar ataxia type 10, X-linked dystonia parkinsonism, Myotonic dystrophy type 2, Autism spectrum disorder associated with fragile site FRA7A, Fuchs endothelial corneal dystrophy, Friedreich ataxia, Spinocerebellar ataxia type 36, C9orf72 amyotrophic lateral sclerosis and/or frontotemporal dementia, Spinocerebellar ataxia type 31, Familial adult myoclonic epilepsy 1, Familial adult myoclonic epilepsy 2, Familial adult myoclonic epilepsy 3, Familial adult myoclonic epilepsy 4, Familial adult myoclonic epilepsy 6, Familial adult myoclonic epilepsy 7, Spinocerebellar ataxia type 37, Dentatorubropallidoluysian atrophy, Huntington disease, Spinal and bulbar muscular atrophy, Spinocerebellar ataxia type 1, Spinocerebellar ataxia type 2, Spinocerebellar ataxia type 3, Spinocerebellar ataxia type 6, Spinocerebellar ataxia type 7, Spinocerebellar ataxia type 17, Pseudoachondroplasia and multiple epiphyseal dysplasia, Blepharophimosis, ptosis and epicanthus inversus syndrome, Cleidocranial dysplasia, Congenital central hypoventilation syndrome, Hand-foot-genital syndrome, Holoprosencephaly 5, Oculopharyngeal muscular dystrophy, Synpolydactyly 1, X-linked hypopituitarism, X-linked intellectual disability, Spinocerebellar ataxia type 8, Myotonic dystrophy type 1, Huntington disease-like 2.

The repeat expanded disease may be a neurological disease, such as Huntington disease-like 2, C9orf72 amyotrophic lateral sclerosis and/or frontotemporal dementia, fragile X-associated tremor/ataxia syndrome, fragile X-associated primary ovarian insufficiency, spinocerebellar ataxia type 10, spinocerebellar ataxia type 31, or spinocerebellar ataxia type 36.

The repeat expanded disease may be myotonic dystrophy type 1, Fuchs endothelial corneal dystrophy, Myotonic dystrophy type 2, Huntington disease-like 2, C9orf72 amyotrophic lateral sclerosis and/or frontotemporal dementia, Fragile X-associated tremor/ataxia syndrome, Fragile X-associated primary ovarian insufficiency, Spinocerebellar ataxia type 36, Spinocerebellar ataxia type 31, or Spinocerebellar ataxia type 10. The inventors found that treatment of these diseases may be particularly effective using a dCas13b protein, such as Psp-dCas13b, as described herein. Hence, the invention provides a method of treating or preventing myotonic dystrophy type 1, Fuchs endothelial corneal dystrophy, Myotonic dystrophy type 2, Huntington disease-like 2, C9orf72 amyotrophic lateral sclerosis and/or frontotemporal dementia, Fragile X-associated tremor/ataxia syndrome, Fragile X-associated primary ovarian insufficiency, Spinocerebellar ataxia type 36, Spinocerebellar ataxia type 31, or Spinocerebellar ataxia type 10, comprise administering to the subject a therapeutically effective amount of the dCas13b protein (e.g., Psp-dCas13b) and a crRNA specific for the dCas13b protein, as described herein.

The repeat expanded disease may be myotonic dystrophy type 1, myotonic dystrophy type 2 or Fuchs endothelial corneal dystrophy. The inventors found that treatment of these diseases may be particularly effective using a dCas13b protein, such as Psp-dCas13b, as described herein.

The repeat expanded disease may be myotonic dystrophy type 1. The inventors found that treatment of these diseases may be particularly effective using a dCas13b protein, such as Psp-dCas13b, as described herein.

The repeat expansion disease may be type I myotonic dystrophy (DM1), the repeat expanded sequences may be (CTG)n, the target RNA may be DMPK1.

Hence, the invention provides a method of treating or preventing DM1, comprising administering to a subject a therapeutically effective amount of the dCas13 protein (e.g., dCas13b, such as Psp-dCas13b) and crRNA described herein, wherein the crRNA comprises a spacer that is complementary to a repeat expanded sequence (CTG)n in DMPK1.

The invention also provides a dCas13 protein (e.g., dCas13b, such as Psp-dCas13b) described herein for use in a method of treating or preventing DM1, wherein the method comprises administering to the subject a therapeutically effective amount of the dCas13 protein (e.g., dCas13b, such as Psp-dCas13b) and a crRNA, wherein the crRNA comprises a spacer that is complementary to a repeat expanded sequence (CTG)n in DMPK1. Similarly, the invention also provides the use of a dCas13 protein (e.g., dCas13b, such as Psp-dCas13b) described herein in the preparation of a medicament for a method of treating or preventing DM1, wherein the method comprises administering to the subject a therapeutically effective amount of the dCas13 protein (e.g., dCas13b, such as Psp-dCas13b) and a crRNA, wherein the crRNA comprises a spacer that is complementary to a repeat expanded sequence (CTG)n in DMPK1.

The invention also provides a crRNA described herein for use in a method of treating or preventing DM1, wherein the method comprises administering to the subject a therapeutically effective amount of a dCas13 protein (e.g., dCas13b, such as Psp-dCas13b) and the crRNA, wherein the crRNA comprises a spacer that is complementary to a repeat expanded sequence (CTG)n in DMPK1. Similarly, the invention also provides the use of a crRNA described herein in the preparation of a medicament for a method of treating or preventing DM1, wherein the method comprises administering to the subject a therapeutically effective amount of a dCas13 protein (e.g., dCas13b, such as Psp-dCas13b) and the crRNA, wherein the crRNA comprises a spacer that is complementary to a repeat expanded sequence (CTG)n in DMPK1.

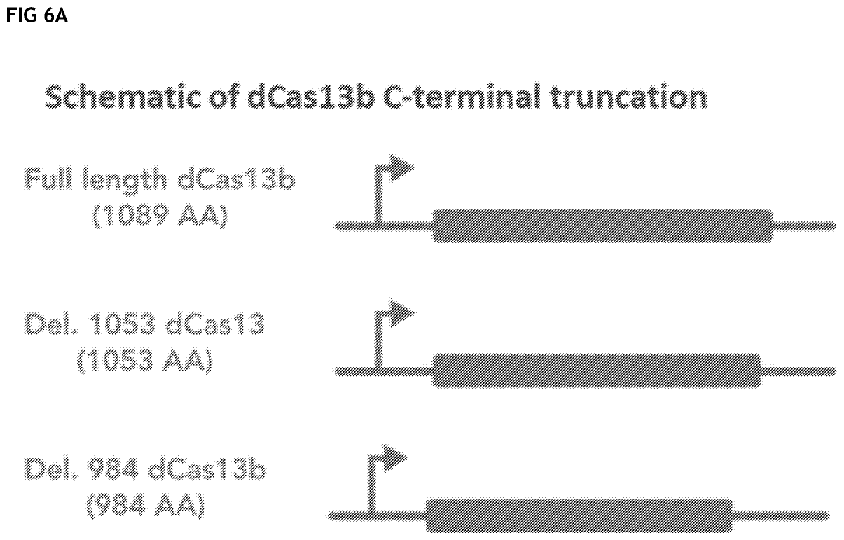

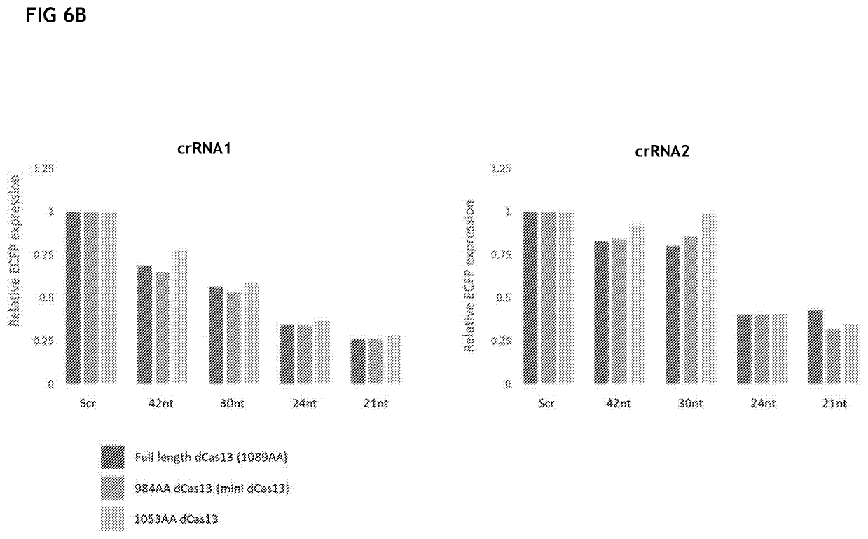

The dCas13 protein may comprise or consist of SEQ ID NOs: 1, 2, or 3. The crRNA may comprise of consist of any one of SEQ ID NOs: 8 to 11 (e.g., see FIGS. 4 and 6).

miRNA Dysregulation-Related Diseases

The methods and uses of the invention may relate to treating or preventing diseases associated with miRNA dysregulation. The inventors have demonstrated that Psp-dCas13b can be programmed using crRNA to block the target sites of miR-17, miR-92a, and miR-222 in a fluorescent reporter system in human cells. Hence, the dCas13 protein and crRNA described herein are particularly useful in blocking the interaction between miRNA and its target binding site, such that miRNA-mediated silencing of target RNA is reduced, thereby treating or preventing diseases associated with miRNA dysregulation.

It has been previously reported that several miRNA-targeted therapeutics (antimiRs) have reached clinical development, including short antisense oligonucleotides targeted at miR-122, which reached phase II trials for treating hepatitis C. The dCas13 and crRNA described herein is more advantageous. Rather than targeting the miRNA, the dCas13 and crRNA described herein prevents the interaction between the miRNA and its binding site by sterically blocking the miRNA binding site within the target RNA, enabling a more precise perturbation compared to miRNA-targeting therapeutics.

Hence, the invention provides a method of treating or preventing a disease associated with miRNA dysregulation, comprising administering to the subject a therapeutically effective amount of the dCas13 protein (e.g., a dCas13b protein, such as Psp-dCas13b) and crRNA described herein. The invention also provides a dCas13 protein described herein for use in a method of treating or preventing a disease associated with miRNA dysregulation, wherein the method comprises administering to the subject a therapeutically effective amount of the dCas13 protein (e.g., a dCas13b protein, such as Psp-dCas13b) and a crRNA. The invention also provides a crRNA described herein for use in a method of treating or preventing a disease associated with miRNA dysregulation, wherein the method comprises administering to the subject a therapeutically effective amount of a dCas13 protein (e.g., a dCas13b protein, such as Psp-dCas13b) and the crRNA. Similarly, the invention also provides the use of a dCas13 protein (e.g., a dCas13b protein, such as Psp-dCas13b) described herein in the preparation of a medicament for a method of treating or preventing a disease associated with miRNA dysregulation, wherein the method comprises administering to the subject a therapeutically effective amount of the dCas13 protein (e.g., a dCas13b protein, such as Psp-dCas13b) and a crRNA. The invention also provides the use of a crRNA described herein in the preparation of a medicament for a method of treating or preventing a disease associated with miRNA dysregulation, wherein the method comprises administering to the subject a therapeutically effective amount of a dCas13 protein (e.g., a dCas13b protein, such as Psp-dCas13b) and the crRNA. The method may comprise modulating the function of a regulatory element in a nucleic acid according to the method of the invention, or modulating the availability, expression and/or activity of a nucleic acid or protein of interest according to the method of the invention. The crRNA may comprise a spacer that is fully complementary or partially complementary to a miRNA-binding site in a target RNA. The crRNA may comprise a spacer that binds to a miRNA neighbouring sequence to disrupt the miRNA-binding site.

The disease associated with miRNA dysregulation may be hepatitis C (dCas13 protein can be used to treat hepatitis C by blocking miR122::HCV interactions), melanoma (dCas13 protein can be used to treat melanoma by blocking miR16::TYRP1), breast cancer (dCas13 protein can be used to treat breast cancer by blocking miR21::PDCD4 and miR21::PTEN), and cardiac failure (dCas13 protein can be used to treat cardiac failure by blocking miR21::Spry1).

### Diseases Treatable by Translational Inhibition

The methods and uses of the invention may relate to treating or preventing diseases associated with dysregulated translation and/or treating or preventing diseases treatable by inhibiting translation. The inventors have demonstrated that Psp-dCas13b can be used to block the translation of fluorescent reporter protein in human cells. Hence, the dCas13 protein and crRNA described herein are particularly useful in inhibiting the translation in aberrantly expressed genes with a toxic gain of function, thereby treating or preventing diseases associated with dysregulated translation and/or treating or preventing diseases treatable by inhibiting translation.

Hence, the invention provides a method of treating or preventing a disease associated with dysregulated translation and/or treating or preventing a disease treatable by inhibiting translation, comprising administering to the subject a therapeutically effective amount of the dCas13 protein (e.g., a dCas13b protein, such as Psp-dCas13b) and crRNA described herein. The invention also provides a dCas13 protein described herein for use in a method of treating or preventing a disease associated with dysregulated translation and/or treating or preventing a disease treatable by inhibiting translation, wherein the method comprises administering to the subject a therapeutically effective amount of the dCas13 protein (e.g., a dCas13b protein, such as Psp-dCas13b) and a crRNA. The invention also provides a crRNA described herein for use in a method of treating or preventing a disease associated with dysregulated translation and/or treating or preventing a disease treatable by inhibiting translation, wherein the method comprises administering to the subject a therapeutically effective amount of a dCas13 protein (e.g., a dCas13b protein, such as psp-dCas13b) and the crRNA. Similarly, the invention also provides the use of a dCas13 protein (e.g., a dCas13b protein, such as Psp-dCas13b) described herein in the preparation of a medicament for a method of treating or preventing a disease associated with dysregulated translation and/or treating or preventing a disease treatable by inhibiting translation, wherein the method comprises administering to the subject a therapeutically effective amount of the dCas13 protein (e.g., a dCas13b protein, such as Psp-dCas13b) and a crRNA. The invention also provides the use of a crRNA described herein in the preparation of a medicament for a method of treating or preventing a disease associated with dysregulated translation and/or treating or preventing a disease treatable by inhibiting translation, wherein the method comprises administering to the subject a therapeutically effective amount of a dCas13 protein (e.g., a dCas13b protein, such as Psp-dCas13b) and the crRNA. The method may comprise modulating the function of a regulatory element in a nucleic acid according to the method of the invention, or modulating the availability, expression and/or activity of a nucleic acid or protein of interest according to the method of the invention. The crRNA may comprise a spacer that is complementary to translation regulatory element, such as a Kozak sequence, a 5′UTR sequence upstream of a start codon of an open reading frame, a start codon of an open reading frame, or a start codon of an upstream open reading frame (uORF) in a target RNA which exhibits a toxic gain of function.

The disease associated with dysregulated translation may be Huntington's disease or amyotrophic lateral sclerosis. The disease treatable by inhibiting translation may be familial hypercholesterolemia.

### Diseases Treatable by Splicing Modulation

The methods and uses of the invention may relate to treating or preventing diseases associated with abnormal splicing and/or treating or preventing diseases treatable by modulating splicing.

The inventors have demonstrated that Psp-dCas13b can be programmed using crRNA to target specific RNA sequences and sterically block the target RNA from interacting with: (1) ribosomes; (2) the miRNA silencing complex; and (3) RNA-binding proteins (see Examples). These data demonstrate that Psp-dCas13b has a sufficiently strong affinity to block the interaction between RNA molecules and various RNA-binding proteins and/or ribonucleoprotein complexes.

RNA splicing is a process where small nuclear ribonucleoproteins (snRNP) bind to a pre-mRNA, form an intronic loop, and remove the intron to produce a mature RNA transcript. Previous studies have demonstrated that splice switching can be induced by sterically blocking an snRNP binding site, for example, with antisense oligonucleotide. Hence, the dCas13 protein and the crRNA described herein may be used to target and sterically block an snRNP binding site can block the interaction between the snRNP and the pre-mRNA, thus inducing splice switching.

Hence, the invention provides a method of treating or preventing a disease associated abnormal splicing and/or a treating or preventing a disease treatable by splicing modulation, comprising administering to the subject a therapeutically effective amount of the dCas13 protein (e.g., a dCas13b protein, such as Psp-dCas13b) and crRNA described herein. The invention also provides a dCas13 protein described herein for use in a method of treating or preventing a disease associated with abnormal splicing and/or a treating or preventing a disease treatable by splicing modulation, wherein the method comprises administering to the subject a therapeutically effective amount of the dCas13 protein (e.g., a dCas13b protein, such as Psp-dCas13b) and a crRNA. The invention also provides a crRNA described herein for use in a method of treating or preventing a disease associated with abnormal splicing and/or a treating or preventing a disease treatable by splicing modulation, wherein the method comprises administering to the subject a therapeutically effective amount of a dCas13 protein (e.g., a dCas13b protein, such as Psp-dCas13b) and the crRNA. Similarly, the invention also provides the use of a dCas13 protein (e.g., a dCas13b protein, such as Psp-dCas13b) described herein in the preparation of a medicament for a method of treating or preventing a disease associated with abnormal splicing and/or a treating or preventing a disease treatable by splicing modulation, wherein the method comprises administering to the subject a therapeutically effective amount of the dCas13 protein (e.g., a dCas13b protein, such as Psp-dCas13b) and a crRNA. The invention also provides the use of a crRNA described herein in the preparation of a medicament for a method of treating or preventing a disease associated with abnormal splicing and/or a treating or preventing a disease treatable by splicing modulation, wherein the method comprises administering to the subject a therapeutically effective amount of a dCas13 protein (e.g., a dCas13b protein, such as Psp-dCas13b) and the crRNA. The method may comprise modulating the function of a regulatory element in a nucleic acid according to the method of the invention, or modulating the availability, expression and/or activity of a nucleic acid or protein of interest according to the method of the invention. The crRNA may comprise a spacer that is complementary to a splicing element, such as a snRNP binding site, exonic splicing enhancer (ESE), and exonic splicing silencer (ESS).

The diseases associated with abnormal splicing may be spinal muscular atrophy (SMA). The disease treatable by modulating splicing may be DMD.

The invention also provides kits and articles of manufacture for use with the invention. The kit may comprise a dCas13 protein, a crRNA, a polynucleotide, a vector, a delivery vehicle, a host cell, a composition or a pharmaceutical composition described herein and instructions for use. The kit may further comprise one or more additional reagents, such as buffers necessary for the makeup and delivery of the dCas13 protein, crRNA, polynucleotide, vector, delivery vehicle, host cell, composition or pharmaceutical composition. The kit may further comprise package inserts with instructions for use.

### EMBODIMENTS

1. A method of modulating the function of a regulatory element in a target RNA, comprising delivering to a cell:
   a catalytically inactive Cas13 protein (dCas13), wherein the Cas13 protein is a member of the Cas13b family, Cas13X family, Cas13Y family or Cas13bt family; and
   a CRISPR RNA (crRNA) capable of recruiting the dCas13 protein to the regulatory element, such that the function of the regulatory element is modulated.
2. The method of embodiment 1, wherein the regulatory element is: (a) a cis-regulatory element, such as a splice element, or (b) a trans-regulatory element, such as a protein binding site or a nucleic acid binding site.
3. The method of embodiment 2, wherein:
   (a) the protein binding site is an expanded repeat sequence, a start codon of an open reading frame, a start codon of an upstream open reading frame (uORF), or a snRNP binding site; or
   (b) the nucleic acid binding site is a miRNA binding site.
4. The method of any one of the preceding embodiments, wherein the Cas13 protein is:
   (a) PspCas13b, Pgu13b, PgiCas13b, Cas13X.1, Cas13X.2, PinCas13b, Cas13Y.1, Cas13Y.2, Cas13Y.3, Cas13bt1, or Cas13bt3;
   (b) PspCas13b, PguCas13b, PgiCas13b, Cas13X.1, Cas13X.2, PinCas13b, Cas13Y.1, Cas13Y.2, Cas13bt1, or Cas13bt3; or
   (c) PspCas13b.
5. The method of embodiment 4, wherein the Cas13 protein is PspCas13b.
6. The method of any one of the preceding embodiments, wherein the dCas13 protein comprises mutation in one or more amino acid residues in a RxxxxH motif of an HEPN-1 domain and a RxxxxH motif of an HEPN-2 domain relative to a corresponding unmodified Cas13 protein.
7. The method of any one of the preceding embodiments, wherein the dCas13 protein comprises or consists of an amino acid sequence having a sequence identity of ≥85% to SEQ ID NO: 1, provided that the amino acid residues corresponding to the amino acid residues at positions 133 and 1058 of SEQ ID NO: 1 are not histidine, e.g., they are both alanine.
8. The method of any one of the preceding embodiments, wherein the dCas13 protein is truncated by <110 amino acid residues from the C-terminus compared to the unmodified protein, and optionally wherein the Cas13 protein of the dCas13 protein is a member of the Cas13b family, such as PspCas13b.
9. The method of embodiment 8, wherein the dCas13 protein comprises or consists of an amino acid sequence having a sequence identity of ≥85% to SEQ ID NO: 2 or 3, provided that the amino acid residue corresponding to the amino acid residue at position 133 of SEQ ID NO: 2 or 3 is not histidine, e.g., it is alanine.
10. The method of any one of the preceding embodiments, wherein the crRNA comprises a dCas13-specific direct repeat and a spacer which is capable of specifically hybridizing with the target RNA sequence.
11. The method of embodiment 10, wherein the crRNA comprises a dCas13b-specific direct repeat and the spacer has a length of between 12 to 36 nucleotides, 12 to 27 nucleotides, or 18 to 24 nucleotides, e.g., the crRNA comprises or consists of any of SEQ ID NOs: 4, 7 to 11, or 73 to 80.
12. A method of modulating the availability, expression and/or activity of a nucleic acid or protein of interest, comprising modulating the function of a regulatory element in a target RNA according to the method of any one of the preceding embodiments, wherein the target RNA encodes or regulates the nucleic acid or protein of interest, such that the availability, expression and/or activity of the nucleic acid or protein of interest is increased or decreased.
13. A method of blocking a miRNA-binding site, comprising modulating the function of a regulatory element in a target RNA according to the method of any one of embodiments 1 to 11, or modulating the availability, expression and/or activity of a nucleic acid or protein of interest according to the method of embodiment 12, wherein the crRNA comprises a spacer that is complementary to the miRNA-binding site, such that miRNA-mediated silencing of the target RNA is reduced.
14. A method of blocking ribosomal attachment or translation of a target RNA, comprising modulating the function of a regulatory element in a nucleic acid according to the method of any one of embodiments 1 to 11, or modulating the availability, expression and/or activity of a nucleic acid or protein of interest according to the method of embodiment 12, wherein the crRNA comprises a spacer that is complementary to a start codon of an open reading frame or a start codon of an upstream open reading frame (uORF), such that translation of the target RNA is reduced.
15. A method of inducing splice switching, comprising modulating the function of a regulatory element in a target RNA according to the method of any one of embodiments 1 to 11, or modulating the availability, expression and/or activity of a nucleic acid or protein of interest according to the method of embodiment 12, wherein the crRNA comprises a spacer that is complementary to a splicing element, such as a RNP-binding site, such that splicing of the target RNA is modulated.
16. A crRNA specific for dCas13b comprising: (i) a dCas13b-specific direct repeat, and (ii) a spacer which is capable of specifically hybridizing with the target RNA sequence and having length of between 18 to 24 nucleotides.
17. A dCas13b protein consisting of an amino acid sequence having a sequence identity of ≥85% to SEQ ID NO: 2 or 3, provided that the amino acid residues corresponding to the amino acid residue at position 133 of SEQ ID NO: 2 or 3 is alanine.
18. A polynucleotide or vector encoding the crRNA according to embodiment 16 or the dCas13b protein according to embodiment 17, optionally wherein the vector is AAV or lentivirus.
19. A delivery vehicle comprising the crRNA according to embodiment 16, the dCas13b protein according to embodiment 17, or the polynucleotide or vector according to embodiment 18.
20. A pharmaceutical composition comprising: (i) the crRNA according to embodiment 16 or the dCas13b protein according to embodiment 17, (ii) and a pharmaceutically acceptable carrier.
21. The crRNA according to embodiment 16 or the dCas13b protein according to embodiment 17, for use in a method of therapy practised on the human or animal body.
22. The crRNA according to embodiment 16, for use in a method of treating a repeat expansion disease, optionally wherein the expansion disease is type 1 myotonic dystrophy (DM1), myotonic dystrophy type 2 or Fuchs endothelial corneal dystrophy.
23. A method of treating or preventing a repeat expansion disease in a subject, wherein the method comprises administering to a subject a therapeutically effective amount of a dCas13 protein and crRNA, wherein the method comprises modulating the function of a regulatory element in a nucleic acid according to the method of any one of embodiments 1 to 11, or modulating the availability, expression and/or activity of a nucleic acid or protein of interest according to the method of embodiment 12, wherein the crRNA comprises a spacer complementary to an expanded repeat sequence, and optionally wherein the expansion disease is type 1 myotonic dystrophy (DM1), myotonic dystrophy type 2 or Fuchs endothelial corneal dystrophy.
24. A dCas13 protein for use in a method of treating a repeat expansion disease, wherein the method comprises administering to a subject therapeutically effective amount of the dCas13 protein and a crRNA, wherein the method comprises modulating the function of a regulatory element in a nucleic acid according to the method of any one of embodiments 1 to 11, or modulating the availability, expression and/or activity of a nucleic acid or protein of interest according to the method of embodiment 12, wherein the crRNA comprises a spacer complementary to an expanded repeat sequence, and optionally wherein the expansion disease is type 1 myotonic dystrophy (DM1), myotonic dystrophy type 2 or Fuchs endothelial corneal dystrophy.
25. The crRNA for use according to embodiment 22, the method of embodiment 23 or the dCas13 protein for use according to embodiment 24, wherein the Cas13 protein is a member of the Cas13b family, and optionally wherein the dCas13 protein comprises or consists of an amino acid sequence having a sequence identity of ≥85% to SEQ ID NO: 2 or 3, provided that the amino acid residue corresponding to the amino acid residue at position 133 of SEQ ID NO: 2 or 3 is alanine.

### Other

It is to be understood that different applications of the dCas13b and crRNA described herein may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural references unless the content clearly dictates otherwise. Thus, for example, reference to “a regulatory element” includes two or more “regulatory elements”.

Furthermore, when referring to “≥x” herein, this means equal to or greater than x. When referred to “≤x” herein, this means less than or equal to x.

For the purpose of this invention, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotide or amino acid residues at each position are then compared. When a position in the first sequence is occupied by the same nucleotide or amino acid as the corresponding position in the second sequence, then the nucleotides or amino acids are identical at that position. The percent identity between the two sequences is a function of the number of identical positions shared by the sequences (i.e., % identity=number of identical positions/total number of positions in the reference sequence×100).

Typically, the sequence comparison is carried out over the length of the reference sequence. For example, if the user wished to determine whether a given (“test”) sequence is 95% identical to SEQ ID NO: 3, SEQ ID NO: 3 would be the reference sequence. To assess whether a sequence is at least 95% identical to SEQ ID NO: 3 (an example of a reference sequence), the skilled person would carry out an alignment over the length of SEQ ID NO: 3, and identify how many positions in the test sequence were identical to those of SEQ ID NO: 3. If at least 95% of the positions are identical, the test sequence is at least 95% identical to SEQ ID NO: 3. If the sequence is shorter than SEQ ID NO: 3, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

All publications, patents and patent applications cited herein, whether supra or infra, are hereby incorporated by reference in their entirety.

The following examples illustrate the invention.

### EXAMPLES

### Example 1

### Material and Methods

Generation of dCas13-Encoding Vectors

All dCas13-encoding vectors were generated by ligating PCR amplicons containing the appropriate dCas13 orthologues, localization signals, and fluorescent markers into a vector containing either an EF1-alpha promoter or a CMV promoter. Amplification conditions were as follows: 98° C. for 60 s, 40 cycles of 98° C. for 10 s, optimal annealing conditions as determined by the NEB Tm calculator for 30 s, 72° C. for 60-180 s, then 5 min at 72° C.

Generation of crRNA-Encoding Vectors

All crRNA-encoding vectors were generated by annealing two single-stranded oligonucleotides and ligating the resulting double-stranded oligonucleotides into a vector containing a hU6 promoter and a pol III stop signal. Custom single-stranded oligonucleotides were synthesized and used in an oligonucleotide annealing reaction. For all oligonucleotide annealing reactions, 10 μL containing 10 M each of the forward and reverse oligonucleotides, 1×T4 DNA ligase buffer, and five units of T4 Polynucleotide Kinase were incubated at 37° C. for 30 min, ramped to 95° C. for 5 min then cooled to 25° C. at a rate of 0.1° C./s. The annealed oligonucleotides were then used as inserts for cloning reactions where applicable.

### Transfection of HEK293T Cells

For each well, 500 ng of dCas13-encoding plasmid, 1 μg of crRNA-encoding plasmid, and 50 ng of bidirectional reporter plasmid were mixed and brought up to 50 l in Opti-MEM™ containing 1.5 μg PEI. The solutions were then mixed and added to the cells. Cells were then incubated for 24 h at 37° C. 5% CO₂. Transfection media was then removed and replaced with fresh DMEM+10% FBS and the cells left for an additional 24 h (48 h from the start of transfection), before being harvested for the flow cytometry analyses.

### Flow Cytometry

Adherent HEK293T cells were harvested using 0.05% Trypsin-EDTA. Following trypsinization, cells were washed with PBS and resuspended in growth medium and were passed through a 70 m cell strainer before flow cytometry analyses were performed. Flow cytometry was performed using BD LSRFortessa™ cell analyzer. ECFP was measured following 405 nm excitation with a 450/50 bandpass filter. EGFP was measured using a 488 nm excitation with a 530/30 bandpass filter. mIFP was measured using a 640 nm excitation with a 670/14 bandpass filter.

### Electroporation and Differentiation of Human Myoblast Cells

First, human myoblast cells were harvested using 0.05% Trypsin-EDTA and were electroporated with Neon Transfection System kit according to the manufacturer's instruction. For every 150,000 cells, 100 ng of Super PiggyBac Transposase Expression Vector and 600 ng of dCas13-crRNA vector were used. The electroporation settings were 1750 V, 10 ms, and 3 pulses. Electroporated cells were then plated and cultured to allow the differentiation of the myoblast cells into myotubes.

### Fluorescent-Activated Cell Sorting (FACS) of Human Myotube Cells

Adherent human myotube cells were harvested using 0.05% Trypsin-EDTA. Following trypsinization, cells were washed with PBS and resuspended in SkMC growth medium supplemented with its SupplementMix and 0.1 g/mL DAPI. Cell suspensions were then passed through a 70 μm cell strainer. Cell sorting experiments were performed using BD FACSAria™ Fusion cell sorter. DAPI was measured following 405 nm excitation with a 450/50 bandpass filter. EGFP was measured using a 488 nm excitation with a 530/30 bandpass filter. Cells were sorted into SkMC growth medium and then immediately brought to the subsequent RNA extraction step.

### RNA Extraction and Semi-Quantitative PCR Analyses

Total RNAs are extracted either from the human myotube tissue cultures or from HSA^{LR} mice. For in vivo experiments, TA muscles harvested from HSA^{LR} mice were homogenized in 1-thioglycerol/homogenization solution for 2×1 min. Subsequently, total RNAs were isolated. Collected total RNAs, either from in vitro or in vivo experiments, were then treated to remove residual plasmid contamination. For each sample, cDNA was generated from 75 ng of total RNA. Subsequently, 1 μL of cDNA preparation was used in a semi-quantitative PCR analysis. Between 1-10 μL of PCR products were then ran on a 2% agarose gel and nucleic acids visualized with GelRed® (Biotium). Gel images were taken using a BioRad GelDoc™ XR+ imager, with exposure times just below what would give saturated pixels (˜0.75 s). Image processing was performed using ImageJ. First, background subtraction was performed, and upper-band and lower-band density were measured for each sample. Then, relative inclusion level (PSI, Ψ) on the exon-skipping events was calculated as the proportion of the upper-band density to the total band density on each gene of interest.

Synthesis of dCas13-Encoding AAV, Transgenic Mice, and AAV Injections

An ultra-purified AAV9 encoding either full length Psp-dCas13b and its 23 nt crRNA; truncated Psp-dCas13b and its 21 nt crRNA; or EGFP were synthesized at VectorBuilder. All mice used in this study are either the FVB-WT strain or the HSA^{LR} transgenic mice with >200 repeats. For TA injections, AAV9 containing either full-length Psp-dCas13b and its 23 nt crRNA or truncated Psp-dCas13b and its 21 nt was diluted in sterile phosphate-buffered saline (PBS) and injected (30 L) into the TA muscle of HSA^{LR} anaesthetized mice (aged 5-9 weeks).

### Example 2

Psp-dCas13b can be Repurposed into a Potent RNA Steric Blocker

The inventors first sought to identify the best Cas13 orthologue to be catalytically inactivated and repurposed as an RNA steric blocker.

LwaCas13a, PspCas13b and RfxCas13d, representing members of each major Cas13 family were selected. Catalytically inactive LwaCas13a (Lwa-dCas13a) was designed by substituting the arginine residues of both RxxxxH motifs of LwaCas13a with alanine. Catalytically inactive PspCas13b (Psp-dCas13b) was designed by substituting the histidine residues of both RxxxxH motifs of PspCas13b with alanine. Catalytically inactive RfxCas13d (Rfx-dCas13d) was designed by substituting the first and last residues of both RxxxxH motifs of RfCas13d with alanine.

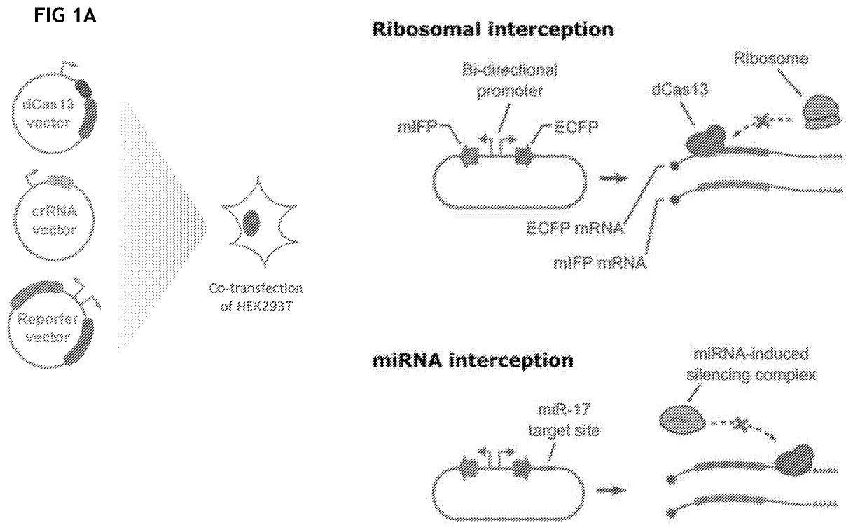

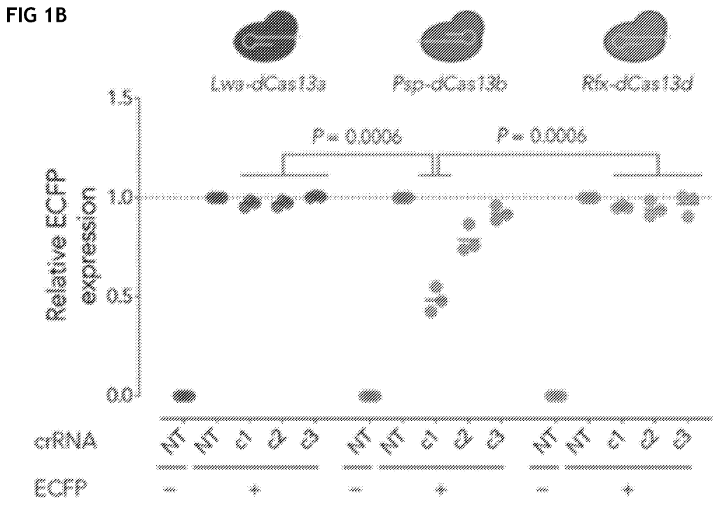

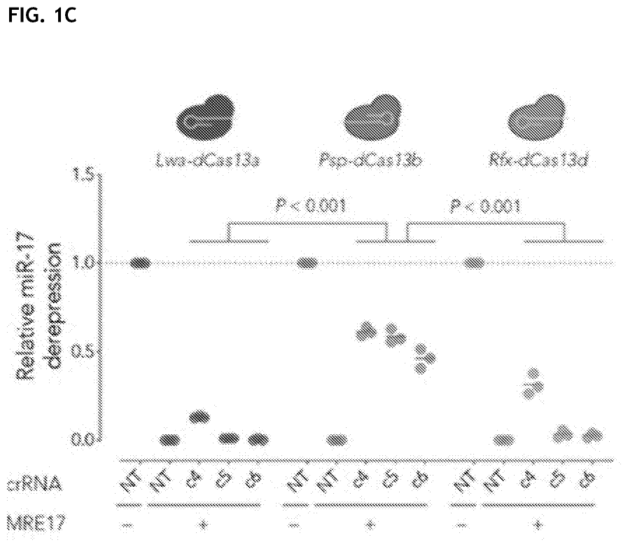

Lwa-dCas13a, Psp-dCas13b, and Rfx-dCas13d were individually cloned into an expression vector containing a sequence encoding an N-terminus nuclear export signal (NES). Then, HEK293T cells were co-transfected with (a) expression vectors encoding NES-Lwa-dCas13a, NES-Psp-dCas13b or NES-Rfx-dCas13d; (b) expression vectors encoding the corresponding crRNA; and (c) expression vectors encoding a bidirectional fluorescent reporter expressing ECFP and mIFP (see FIG. 1A). The relative expression level of ECFP to mIFP was quantified by flow cytometry to determine the blocking efficiency of each dCas13 orthologue.

To compare the ribosomal-blocking efficiency of Lwa-dCas13a, Psp-dCas13b and Rfx-dCas13d, these dCas13 proteins were programmed using their corresponding crRNAs to bind and sterically block the 5′ UTR or translational start site (TSS) of the ECFP transgene (see FIG. 1A, top right). Meanwhile, to compare the miRNA-blocking efficiency of Lwa-dCas13a, Psp-dCas13b and Rfx-dCas13d, an miR-17 binding site was cloned at the 3′ UTR of the ECFP transgene, which would enable miR-17-induced ECFP downregulation relative to mIFP. The dCas13 proteins were then programmed using their corresponding crRNAs to bind and sterically block the miR-17 target site (FIG. 1A, bottom right).

The inventors observed that Psp-dCas13b exhibited the highest blocking efficiency, leading to ˜50% ECFP repression and miR-17 de-repression in ribosomal- and miRNA-blocking experiments, respectively (FIGS. 1B and C).

### Example 3

Shortening the crRNA Spacer Improves the Steric Blocking Efficiency of Psp-dCas13b

To further improve the steric blocking efficiency of Psp-dCas13b, the inventors tested different lengths of crRNA spacer. The inventors hypothesized that longer spacers which extend Watson-Crick base pairing beyond the native length of 30 nt found in the Prevotella sp. P5-125 transcriptome would increase binding affinity and thus increase blocking efficiency. Surprisingly, shorter spacers of 18-24 nt mediated a remarkably higher blocking efficiency, achieving complete repression of ECFP in the ribosomal-blocking experiment (FIG. 2A).

To confirm that the observed steric blocking effects were mediated by the interaction between Psp-dCas13b and the crRNA ribonucleoprotein (RNP) complex, rather than crRNA:mRNA hybridisation, the inventors performed a florescent reporter experiment that omits either Psp-dCas13b or the crRNA. The result of this experiment showed that both Psp-dCas13b and the crRNA are essential for blocking ECFP translation (FIG. 2B), indicating that crRNA:mRNA hybridisation, alone, is unlikely to play a major role in blocking ribosomal attachment.

### Example 4

Psp-dCas13b Blocks miRNA Interactions in a Highly Specific Manner

To explore the general applicability of Psp-dCas13b for blocking miRNA functions, the inventors individually cloned three bidirectional reporter vectors which express ECFP and mIFP, and contain the binding site of either miR-17, miR-92a or miR-222 at the 3′ UTR of the ECFP transgene. miR-17, miR-92a and miR-222 are known to be highly active in HEK293T cells and were found to repress ECFP expression relative to mIFP (data not shown). Subsequently, a total of 45 crRNA vectors were designed to tile the whole 3′ UTR of the ECFP transcript.

The inventors observed that co-expression of Psp-dCas13b and its corresponding crRNA led to de-repression of ECFP expression in all three miRNA reporters, indicating a generalizable nature of Psp-dCas13b as a miRNA blocker. In particular, the inventors demonstrated that de-repression was only observed when Psp-dCas13b was guided by crRNAs that are designed to partially or completely cover the miRNA binding site.

Meanwhile, when Psp-dCas13b was targeted by crRNAs comprising spacers that are complementary to the neighbouring region of the miRNA binding sites, no consistent de-repression was observed (FIG. 3A).

The inventors further measured the extent of de-repression achieved by the Psp-dCas13b blocker by comparing the relative ECFP expression level with one that is achieved with miRNA-blocking antisense oligonucleotide (antagomiR) treatment (FIG. 3B). The inventors observed that blocking the miR-17 target site with Psp-dCas13b phenocopies the de-repression level achieved with antagomiR-17, causing a complete blocking of miRNA activity against ECFP mRNA.

### Example 5

Targeting the Repeat Expansion Sequence in DMPK1 mRNA with Psp-dCas13b Reverses Disease Pathology

Upon establishing the applicability of Psp-dCas13b to block ribosomal- and miRNA-functions, the inventors explored the utility of the system to treat an RNA-dominant disease. Type 1 myotonic dystrophy (DM1) is an inherited neuromuscular condition caused by an expansion of the (CTG)n repeat sequence at the 3′ UTR of the DMPK1 gene. The expanded sequence is subsequently transcribed into a toxic RNA that sequesters cellular splicing factors and produces repeat-associated small peptides (FIG. 4A). Symptoms and signs of DM1 primarily arise from a widespread spliceopathy due to splicing factor sequestration (11, 12). Previous studies have shown that mis-splicing of ATP2A1 and SOS1 mRNA leads to muscle stiffness and myotonia, which constitute the hallmark symptoms of the disease. Mis-splicing of other gene transcripts, such as INSR, cTNT, DMD, MBNL1 and MBNL2, are also known to contribute to the development of other symptoms, such as insulin resistance, cardiac conduction defects, learning difficulties and infertility.

The inventors hypothesized that Psp-dCas13b can be used to treat DM1 by sterically blocking the toxic repeat sequence in the DMPK1 mRNA, therefore de-sequestering the splicing factors and inhibiting repeat associated non-AUG (RAN) translation. To explore this possibility, the inventors electroporated wild-type (WT) or DM1 patient-derived myoblasts with piggyBac plasmids encoding NLS-dCas13b-NLS-P2A-EGFP and its corresponding crRNAs. Five different crRNAs were designed for this experiment which encode a scrambled non-targeting control (Scr), 23 nt-(crRNA1), 31 nt-(crRNA2), 21 nt-(crRNA3) or 24 nt-(crRNA4) repeat targeting sequence (FIG. 4B). Electroporated myoblasts were then differentiated into myotubes for 6 days, after which EGFP(+) cells were sorted. Total RNAs from EGFP(+) cells were then collected and the splicing pattern of six biomarker exons were assessed with RT-PCR. The inventors observed a consistent reversal of the disease-associated splicing pattern across all biomarker transcripts, which include SOS1, INSR, MBNL1, MBNL2, ATP2A1 and DMD. The inventors also observed a varying level of splicing correction associated with different spacer lengths on the crRNA. In line with the inventors' observations described in “Shortening the crRNA spacer improves steric blocking efficiency of Psp-dCas13b”, short spacers of 21 nucleotides (crRNA3), 23 nucleotides (crRNA1) and 24 nucleotides (crRNA4) mediate a complete reversal of splicing, while the 31-nt spacer (crRNA2) only mediates partial corrections (FIGS. 4C and D). The ability of crRNA3 to reverse pathological splicing patterns was further evidenced by RNAseq (FIGS. 4E and F).

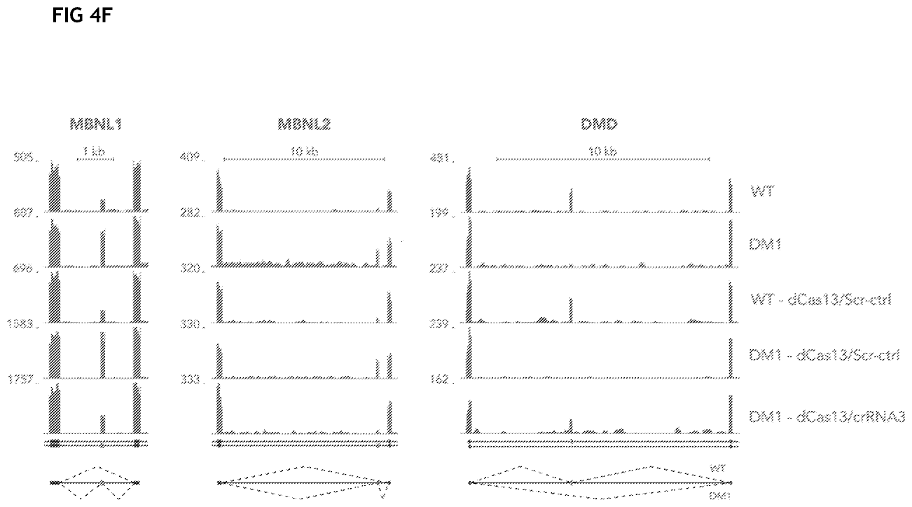

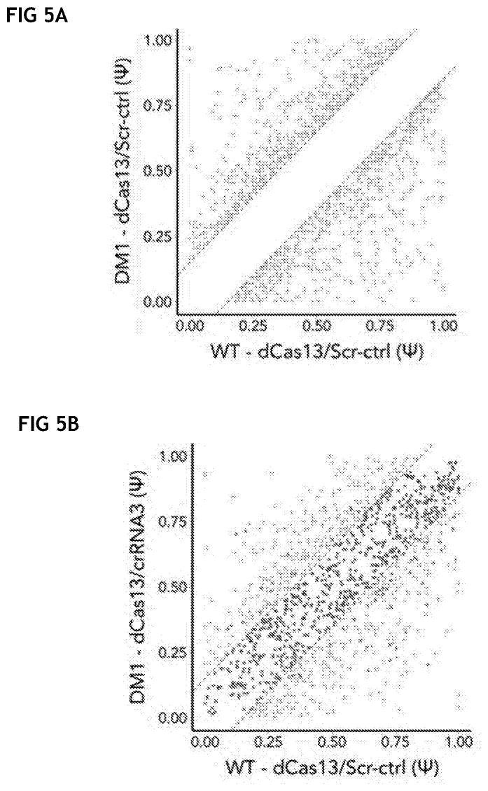

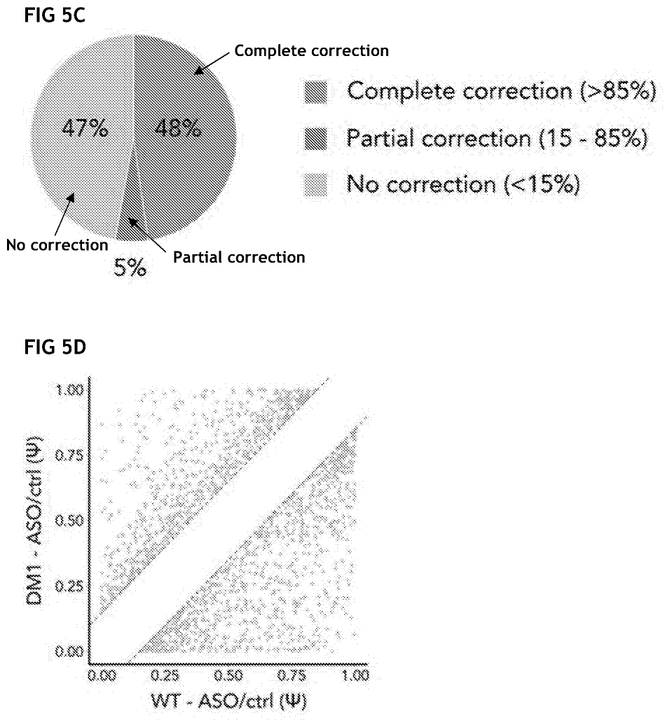

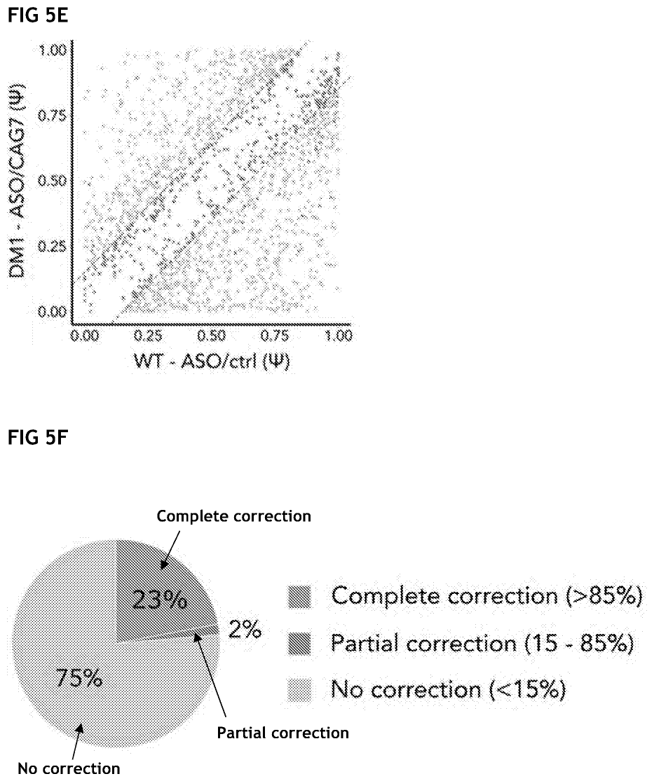

The inventors further compared the extent of reversal of pathological splicing achieved by the dCas13 system with the extend of reversal of pathological splicing achieved by antisense oligonucleotide CAG7. Using RNAseq, the inventors found that the dCas13 system significantly outperformed antisense oligonucleotides. In particular, Psp-dCas13b/crRNA3 treatment of DM1 patient-derived myoblasts with Psp-dCas13b/crRNA3 completely corrected 48% of DM1-related mis-splicing events, whereas treatment with CAG7 only achieved 23% correction (FIG. 5).

### Example 6

C-Terminal Truncation of Psp-dCas13b does not Affect its Steric Blocking Efficiency

To further explore the utility of Psp-dCas13b as a therapeutic modality, the inventors sought to deliver an AAV9-encoded Psp-dCas13b to a mouse model of DM1. While AAV is known to be an efficient vehicle for gene therapy, its packaging capacity is limited to ˜4.7 kb. Therefore, the relatively large size of Psp-dCas13b leaves little room for the inclusion of a promoter or other regulatory sequences to control the expression pattern of Psp-dCas13b. To address this important issue, the inventors designed two C-terminally truncated variants of Psp-dCas13b and benchmarked their steric blocking efficiency against the full-length protein in the fluorescent reporter context (FIG. 6A). The inventors observed that both of the truncated variants of Psp-dCas13b (one of 1053 amino acid residues in length [1053-aa] and one of 984 amino acid residues in length [984-aa]) have comparable ribosomal-blocking efficiencies and sensitivities to spacer length with the full-length variant (FIG. 6B).

The 984-aa variant is henceforth referred to as mini-Psp-dCas13b and used in the subsequent in vivo experiments.

### Example 7

### Psp-dCas13b Reverses Splicing Deregulation in a DM1 Mouse Model

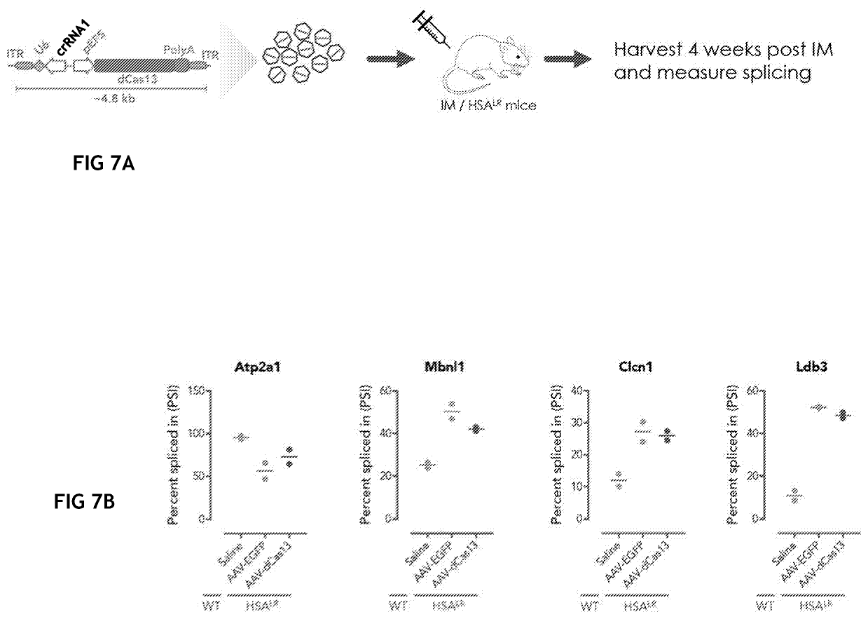

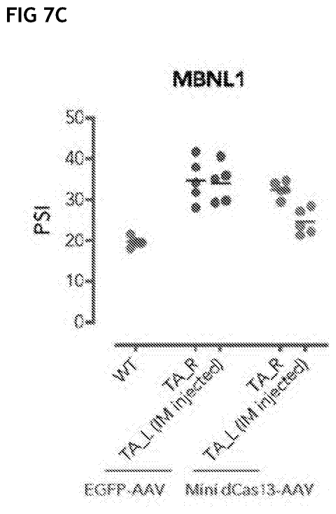

Next, the inventors packaged a therapeutic AAV9 vector encoding: (1) pEFS-driven full-length Psp-dCas13b or pCMV-driven mini-Psp-dCas13b; and (2) phU6-driven 21-nt CAG repeat-containing crRNA. A control AAV9 vector encoding pCMV-driven EGFP was also generated. Experiments were conducted by injecting 8-week-old HSA^{LR} mice with ˜1×10¹² vector genomes (vg) of either the therapeutic or control AAV9 vector in the left tibialis anterior (TA) muscle in two independent experiments (n=5; FIG. 7A). TA muscle tissue was then collected 4 weeks after injection.

The inventors observed a moderate reversal of splicing deregulation of Atp2a1, Mbnl1, C1cn1 and Ldb3 transcripts in mice injected with pEFS-driven full-length Psp-dCas13b. The inventors also observed that the pCMV-driven mini-Psp-dCas13b led to a stronger reversal of splicing deregulation of Mbnl1 transcripts than the pEFS-driven full-length Psp-dCas13b (FIG. 7B), which likely reflects the improved expression level of mini-Psp-dCas13b driven by the CMV promoter.

Intracellular dosage of dCas13 plays a major role in reversing spliceopathy in vitro. Indeed, when the expression level of dCas13 is too high or too low, the therapeutic efficacy of the dCas13 is reduced. Accordingly, the optimal dosage of mini-Psp-dCas13b can be identified by testing a wide range of dosages. For example, a dosage range wherein there is a 1,000-fold difference between the lowest and highest dosage can be tested. At the optimum dosage, the pCMV-driven mini-Psp-dCas13b is expected to cause even stronger reversal of splicing deregulation of Mbnl1, Atp2a1, Cln1 and Ldb3 transcripts in mice as observed in vitro (Example 5 and FIG. 4).

### Example 8

### Psp-dCas13b May Reverse Splicing Deregulation in a Myotonic Dystrophy Type 2 Mouse Model

Given that Psp-dCas13b can reverse splicing deregulation in a DM1 mouse model, it is expected that Psp-dCas13b can also have use in treating other repeat expansion diseases where: (1) repeat expansion occurs in the non-coding region; and (2) somatic instability/RBP sequestration/RAN translation plays some role in disease pathology.

Myotonic dystrophy type 2 (DM2) results from an unstable CCTG tetranucleotide repeat expansion in intron 1 of the CNBP gene. In healthy range alleles, the (CCTG)n repeat tract is generally interrupted by one or more GCTG, TCTG or ACTG motifs. However, in expanded alleles, the repeat tract is typically uninterrupted. Like DM1, the repeat expansion is transcribed into a toxic RNA that sequesters cellular splicing factors (resulting in mis-splicing of various RNA transcripts such as CLCN1, INSR, LDB3, MAPT, TNNT3; reference 13) and produces repeat-associated small peptides.

To investigate the ability of Psp-dCas13b to reverse splicing deregulation in DM2, therapeutic AAV9 can be generated encoding: (1) pCMV-driven mini-Psp-dCas13b; and (2) phU6-driven 24-nt CCTG repeat-containing crRNA. AAV9 vector encoding pCMV-driven EGFP can also be generated. After packaging these AAV9 vectors, experiments can be conducted by giving a DM2 mouse model an intravascular injection with 1×10¹⁰ to 1×10¹² vector genomes (vg) of either the therapeutic or control AAV9 vector. Tissue can then be collected between 6-8 weeks after injection.

Total RNAs from the samples can then be collected and the splicing pattern of biomarkers, e.g., CLCN1, INSR, LDB3, MAPT, and/or TNNT3, can be assessed with RT-PCR. It is expected that only the samples derived from mice injected with the therapeutic vector would show consistent reversal of splicing deregulation.

The AAV9 vector encoding Psp-dCas13b and crRNA would be more advantageous than FDA-approved antisense oligonucleotides because: (a) delivering an AAV9-encoded transgene allows a higher intracellular concentration of the therapeutic molecule to be achieved, whereas delivering a sufficient amount of ASOs into the intracellular compartment has always been challenging; (b) delivering an AAV9-encoded transgene allows the design of a one-off therapeutic strategy, whereas ASO therapy requires routine injection; and (c) the Psp-dCas13b would produce fewer off-target effects due to the sensitivities of the Cas13 system to mismatches compared to ASOs. This would be in line with the comparative data shown in FIG. 5.

### Example 9

### Psp-dCas13b May Promote SMN2 Exon 7 Inclusion in an SMA Mouse Model

Psp-dCas13b has been found to sterically block target RNA sequences from interacting with ribonucleoprotein complexes including ribosomes and miRNA silencing complexes. Therefore, Psp-dCas13b is also likely to have an ability to promote splice switching by blocking interaction between pre-mRNA transcripts and small nuclear ribonucleoproteins (snRNPs).

Spinal muscular atrophy (SMA) results from mutations or deletions of the Survival Motor Neuron 1 (SMN1) gene coupled with predominant skipping of SMN2 exon 7. It is known in the art that a 15-nt sequence in SMN2 intron 7, termed the intronic splicing silencer N1 (ISS-N1) can be targeted using antisense-oligonucleotides to treat SMA.

To investigate the ability of Psp-dCas13b to promote retention of SMN2 exon 7, therapeutic AAV9 vectors can be generated encoding: (1) pCMV-driven mini-Psp-dCas13b; and (2) phU6-driven crRNA with a spacer comprising a sequence complementary to the ISS-N1 of SMN2 intron 7. AAV9 vector encoding pCMV-driven EGFP can also be generated. After packaging these AAV9 vectors, experiments can be conducted by giving an intrathecal injection into an SMA mouse model showing predominant skipping of SMN2 exon 7 with ˜1×10¹⁰-1×10¹² vector genomes (vg) of either the therapeutic or control AAV9 vector. The spinal cord and brain tissue can then be collected 6-8 weeks after injection.

Total RNAs from the samples can then be collected and the splicing pattern of SMN2 can be assessed with RT-PCR. It is expected that only the samples derived from mice injected with the therapeutic vector would show consistent retention of SMN2 exon 7.

The therapeutic vector encoding Psp-dCas13b and cRNA would be more advantageous than FDA-approved antisense oligonucleotides because: (a) delivering an AAV9-encoded transgene allows a higher intracellular concentration of the therapeutic molecule to be achieved, whereas delivering a sufficient amount of ASOs into the intracellular compartment has always been challenging; (b) delivering an AAV9-encoded transgene allows the design of a one-off therapeutic strategy, whereas ASO therapy requires routine injection; and (c) the Psp-dCas13b would produce fewer off-target effects due to the sensitivities of the Cas13 system to mismatches compared to ASOs. This would be in line with the comparative data shown in FIG. 5.

### Example 10

### Psp-dCas13b May Reverse RNA-Binding Protein (RBP) Sequestration and Block RAN Translation in Cellular Model of Fuchs Endothelial Corneal Dystrophy (FECD)

Given the ability of Psp-dCas13b in blocking RNP attachment and reversing RBP sequestration in DM1 cellular and mice model, it is likely that this modality can also mediate the reversal of RBP sequestration and block RAN translation in Fuchs Endothelial Corneal Dystrophy (FECD).

FECD is an ophthalmological disease characterised by progressive loss of corneal endothelial cells, thickening of Descement's membrane, and deposition of extracellular matrix in the form of guttae. It is caused by an expansion of (CTG)n repeat in intron 3 of TCF4 gene. FECD is inherited in an autosomal dominant mode, and similarly with DM1, RBP sequestration and RAN translation have central roles in the pathology of the disease.

To investigate the ability of Psp-dCas13b to reverse RBP sequestration in FECD, therapeutic AAV9 can be generated encoding: (1) pCMV-driven mini-Psp-dCas13b; and (2) phU6-driven 21-nt CTG repeat-targeting crRNA. AAV9 vector encoding pCMV-driven EGFP can also be generated as control. After packaging these AAV9 vectors, experiments can be conducted by transducing patient-derived corneal cell in culture. Around 2-4 days after transduction, cells will be fixed and stained to visualise nuclear foci indicating the presence of RBP sequestration. It is expected that only the samples derived from mini-dCas13b-treated cells would show consistent reversal of RBP sequestration.

### REFERENCES

1 Zhang et al. Front Genet. 11:594576 (2020).
2 Cox et al. Science 358:6366 (2017).
3 Emeric et al., bioRxiv preprint DOI:10.1101/2021.05.26.445687 (2007)
4 Cunningham et al. Science 244, 1081-1085 (1989).
5 Sambrook et al., Molecular Cloning: A Laboratory Manual, CSH Press 1989, pp. 15.3-15.108.
6 Konermann et al., Cell 173:665-676, 2018.
7 Cokol et al., EMBO Rep. 1(5):411-415 (2000).
8 Freitas and Cunha, Curr Genomics 10(8): 550-557 (2009).
9 Slaymaker, I. M. et al. Cell Rep. 26, 3741-3751.e5 (2019).
10 Ausubel (ed), Wiley Interscience, New York and the Maniatis Manual produced by Cold Spring Harbor Publishing (1999).
11 Nakamori et al. Ann Neurol. 74(6):862-72 (2013)
12 Savkur et al. Nat Genet. 29(1):40-7 (2001)
13 Vihola et al. Acta Neuropathol. 119(4): 465-479 (2010)

TABLE 1
Examples of Cas13 proteins.

| | | | Protein Accession/ | Human cell | Nuclease activity |
|---|---|---|---|---|---|
| Family | Orthologue name | Species/Source of sequence | Contig Accession | compatibility | in human cells |
| Cas13a | LshCas13a (SEQ ID NO: 12) | Leptotrichia shahii | WP_018451595.1 | Yes | ++ |
| | LwaCas13a (SEQ ID NO: 13) | Leptotrichia wadei | WP_021746774.1 | Yes | +++ |
| | LseCas13a (SEQ ID NO: 14) | Listeria seeligeri | WP_01298547 7.1 | Unclear | + |
| | LbmCas13a (SEQ ID NO: 15) | Lachnospiraceae bacterium | WP_04492118 8.1 | Yes | +++ |
| | | MA2020 | | | |
| | LbnCas13a (SEQ ID NO: 16) | Lachnospiraceae bacterium | WP_02278544 3.1 | Yes | ++ |
| | | NK4A179 | | | |
| | CamCas13a (SEQ ID NO: 17) | Clostridium aminophilum DSM | WP_03147334 6.1 | Unclear | + |
| | | 10710 | | | |
| | CgaCas13a (SEQ ID NO: 18) | Carnobacterium gallinarum DSM | WP_03456016 3.1 | Unclear | − |
| | | 4847 | | | |
| | Cga2Cas13a (SEQ ID NO: 19) | Carnobacterium gallinarum DSM | WP_03456384 2.1 | Unclear | + |
| | | 4847 | | | |
| | PprCas13a (SEQ ID NO: 20) | Paludibacter propionicigenes | WP_01344371 0.1 | Unclear | + |
| | | WB4 | | | |
| | LweCas13a (SEQ ID NO: 21) | Listeria weihenstephanen sis FSL | WP_03605918 5.1 | Unclear | + |
| | | R9-0317 | | | |
| | Lwa2Cas13a (SEQ ID NO: 22) | Leptotrichia wadei F0279 | WP_02174677 4.1 | Not known | Not known |
| | LbfCas 13a (SEQ ID NO: 23) | Listeriaceae bacterium FSL M6- | WP_03609100 2.1 | Yes | +++ |
| | | 0635 (Listeria newyorkensis) | | | |
| | RcsCas13a (SEQ ID NO: 24) | Rhodobacter capsulatus SB 1003 | WP_01306772 8.1 | Yes | ++ |
| | RcrCas13a (SEQ ID NO: 25) | Rhodobacter capsulatus R121 | WP_02391150 7.1 | Yes | ++ |
| | RcdCas13a (SEQ ID NO: 26) | Rhodobacter capsulatus DE442 | WP_02391150 7.1 | Yes | ++ |
| | LbuCas13a (SEQ ID NO: 27) | Leptotrichia buccalis C-1013-b | WP_015770004.1 | Yes | +++ |
| | HheCas13a (SEQ ID NO: 28) | Herbinix hemicellulosilytica | CRZ35554.1 | Yes | +++ |
| | EreCas13a (SEQ ID NO: 29) | Eubacterium rectale | WP_055061018.1 | Not known | Not known |
| | EbaCas13a (SEQ ID NO: 30) | Eubacteriaceae bacterium | WP_090127496.1 | Unclear | + |
| | | CHKCI004 | | | |
| | BmaCas13a (SEQ ID NO: 31) | Blautia sp. Marseille-P2398 | WP_062808098.1 | Unclear | + |
| | LspCas13a (SEQ ID NO: 32) | Leptotrichia sp. oral taxon 879 | WP_021744063.1 | Yes | ++ |
| | | str. F0557 | | | |
| | LbhCas13a | | | Not known | Not known |
| Cas13b | BzoCas13b (SEQ ID NO: 33) | Bergeyella zoohelcum | WP_002664492 | Yes | ++ |
| | PinCas 13b (SEQ ID NO: 34) | Prevotella intermedia | WP_036860899 | Yes | +++ |
| | PbuCas13b (SEQ ID NO: 35) | Prevotella buccae | WP_004343973 | Yes | ++ |
| | AspCas13b (SEQ ID NO: 36) | Alistipes sp. ZOR0009 | WP_047447901 | Unclear | + |
| | PsmCas13b (SEQ ID NO: 37) | Prevotella sp. MA2016 | WP_036929175 | Yes | ++ |
| | RanCas13b (SEQ ID NO: 38) | Riemerella anatipestifer | WP_004919755 | Yes | ++++ |
| | PauCas 13b (SEQ ID NO: 39) | Prevotella aurantiaca | WP_025000926 | Yes | ++ |
| | PsaCas 13b (SEQ ID NO: 40) | Prevotella saccharolytica | WP_051522484 | Unclear | + |
| | Pin2Cas13b (SEQ ID NO: 41) | Prevotella intermedia | WP_061868553 | Yes | ++ |
| | CcaCas13b (SEQ ID NO: 42) | Capnocytophaga canimorsus | WP_013997271 | Unclear | + |
| | PguCas13b (SEQ ID NO: 43) | Porphyromonas gulae | WP_039434803 | Yes | ++++ |
| | PspCas13b (SEQ ID NO: 44) | Prevotella sp. P5-125 | WP_044065294 | Yes | ++++ |
| | FbrCas13b (SEQ ID NO: 45) | Flavobacterium branchiophilum | WP_014084666 | Unclear | + |
| | PgiCas13b (SEQ ID NO: 46) | Porphyromonas gingivalis | WP_053444417 | Yes | ++++ |
| | Pin3Cas13b (SEQ ID NO: 47) | Prevotella intermedia | WP_050955369 | Yes | ++ |
| Cas13bt | Cas13btl (SEQ ID NO: 48) | [JGI Metagenome] Atlantic deep | Ga0114919_10002421 | Yes | ++ |
| | | subsurface | | | |
| | Cas13bt2 (SEQ ID NO: 49) | [JGI Metagenome] Marine | Ga0209427_10000033 | No | − |
| | | sediment | | | |
| | Cas13bt3 (SEQ ID NO: 50) | [NCBI WGS Metagenome] | QNBS01000103.1 | Yes | ++ |
| | | Planctomycetes bacterium isolate | | | |
| | | B28_G16 (marine sediment) | | | |
| | Cas13bt4 (SEQ ID NO: 51) | [JGI Metagenome] Freshwater | Ga0315296_10033793 | Not known | Not known |
| | | lake sediment | | | |
| | Cas13bt5 (SEQ ID NO: 52) | [JGI Metagenome] Salt marsh | Ga0315532_1006943 | Not known | Not known |
| | | sediment | | | |
| | Cas13bt6 (SEQ ID NO: 53) | [JGI Metagenome] Salt marsh | Ga0315541_1003536 | Not known | Not known |
| | | sediment | | | |
| | Cas13bt7 (SEQ ID NO: 54) | [JGI Metagenome] Groundwater | Ga0246100_107590 | Not known | Not known |
| | Cas13bt8 (SEQ ID NO: 55) | [JGI Metagenome] Salt marsh | Ga0315533_1000464 | Not known | Not known |
| | | sediment | | | |
| | Cas13bt9 (SEQ ID NO: 56) | [JGI Metagenome] Freshwater | Ga0315294_10038294 | Not known | Not known |
| | | lake sediment | | | |
| | Cas13bt10 (SEQ ID NO: 57) | [JGI Metagenome] Salt marsh | Ga0315554_1005387 | Not known | Not known |
| | | sediment | | | |
| | Cas13bt11 (SEQ ID NO: 58) | [JGI Metagenome] Freshwater | Ga0315285_10018775 | Not known | Not known |
| | | lake sediment | | | |
| | Cas13bt12 (SEQ ID NO: 59) | [JGI Metagenome] Salt marsh | Ga0307443_1009138 | Not known | Not known |
| | | sediment | | | |
| | Cas13bt13 | [JGI Metagenome] Salt marsh | Ga0315552_1001799 | Not known | Not known |
| | | sediment | | | |
| | Cas13bt14 (SEQ ID NO: 60) | [JGI Metagenome] Salt marsh | Ga0307431_1000754 | Not known | Not known |
| | | sediment | | | |
| | Cas13bt15 (SEQ ID NO: 61) | [JGI Metagenome] Salt marsh | Ga0315552_1002530 | Not known | Not known |
| | | sediment | | | |
| | Cas13bt16 (SEQ ID NO: 62) | [JGI Metagenome] Salt marsh | Ga0315532_1010951 | Not known | Not known |
| | | sediment | | | |
| Cas13c | FnsCas13c (SEQ ID NO: 63) | Fusobacterium necrophorum | WP_005959231.1 | Unclear | + |
| | | subsp. funduliforme ATCC | | | |
| | | 51357 contig00003 | | | |
| | FndCas13c (SEQ ID NO: 64) | Fusobacterium necrophorum DJ- | WP_035906563.1 | Not known | Not known |
| | | 2 contig0065, whole genome | | | |
| | | shotgun sequence | | | |
| | FnbCas13c (SEQ ID NO: 65) | Fusobacterium necrophorum | WP_035935671.1 | Yes | ++ |
| | | BFTR1 contig0068 | | | |
| | FnfCas13c (SEQ ID NO: 66) | Fusobacterium necrophorum | EHO19081.1 | Unclear | + |
| | | subsp. funduliforme 1_1_36S | | | |
| | | cont1.14 | | | |
| | FpeCas13c (SEQ ID NO: 67) | Fusobacterium perfoetens ATCC | WP_027128616.1 | Not known | Not known |
| | | 29250 | | | |
| | | T364DRAFT_scaffold00009.9_C | | | |
| | FulCas13c (SEQ ID NO: 68) | Fusobacterium ulcerans ATCC | WP_040490876.1 | Not known | Not known |
| | | 49185 cont2.38 | | | |
| | AspCas13c (SEQ ID NO: 69) | Anaerosalibacter sp. ND1 | WP_042678931.1 | Yes | ++ |
| | | genome assembly | | | |
| | | Anaerosalibacter massiliensis | | | |
| | | ND1 | | | |
| Cas13d | EsCas13d | Eubacterium siraeum | n/a | Unclear | + |
| | RffCas13d | Ruminoccocus flavefaciens FD1 | n/a | Unclear | + |
| | UrCas13d | Uncultured Ruminococcus sp. | n/a | Yes | ++ |
| | RaCas13d | Ruminococcus albus | n/a | Unclear | + |
| | P1E0Cas13d | Gut metagenome assembly | n/a | Yes | ++ |
| | | P1E0-k21 | | | |
| | AdmCas13d | Anaerobic digester metagenome | n/a | Yes | +++ |
| | | 15706 | | | |
| | RfxCas13d | Ruminoccocus flavefaciens | n/a | Yes | ++++ |
| | | XPD3002 | | | |
| Cas13x | Cas13X.1 | [JGI Metagenome] marine | Ga0180301_10008484 | Yes | ++++ |
| | | sediment | | | |
| | Cas13X.2 | [JGI Metagenome] salt marsh | Ga0307443_1000411 | Yes | +++ |
| Cas13y | Cas13Y.1 | [JGI Metagenome] salt marsh | Ga0307438_1006815 | Yes | +++ |
| | Cas13Y.2 | [JGI Metagenome] salt marsh | Ga0307438_1004067 | Yes | +++ |
| | Cas13Y.3 | [JGI Metagenome] salt marsh | Ga0307431_1000754 | Yes | ++ |
| | Cas13Y.4 | [JGI Metagenome] Hypersaline | Ga0180434_10014215 | Yes | + |
| | | lake sediment | | | |
| | Cas13Y.5 | [JGI Metagenome] salt marsh | Ga0307443_1000012 | Yes | ++ |

TABLE 2
Repeat expansion diseases and example crRNA spacer sequences

| | Host | Repeat | | Example |
|---|---|---|---|---|
| Disease | gene | motif | Location | crRNA spacer sequence |
| Myotonic dystrophy type 1 | DMPK | CTG | 3′ UTR | 5′-GCAGCAGCAGCAGCAGCAGCA-3′ |
| | | | | (SEQ ID NO: 73) |
| Fuchs endothelial corneal | TCF4 | CTG | Intron | 5′-GCAGCAGCAGCAGCAGCAGCA-3′ |
| dystrophy | | | | (SEQ ID NO: 73) |
| Myotonic dystrophy type 2 | CNBP | CCTG | Intron | 5′-GCAGGCAGGCAGGCAGGCAGG-3′ |
| | | | | (SEQ ID NO: 74) |
| Huntington disease-like 2 | JPH3 | CTG | 3′ UTR | 5′-GCAGCAGCAGCAGCAGCAGCA-3′ |
| | | | | (SEQ ID NO: 75) |
| C9orf72 amyotrophic lateral | C9orf72 | GGGGCC | Intron | 5′-GGCCCCGGCCCCGGCCCCGGCC-3′ |
| sclerosis and/or frontotemporal | | | | (SEQ ID NO: 76) |
| dementia | | | | |
| Fragile X-associated | FMR1 | CGG | 5′ UTR | 5′-GCCGCCGCCGCCGCCGCCGCC-3′ |
| tremor/ataxia syndrome | | | | (SEQ ID NO: 77) |
| Fragile X-associated primary | FMR1 | CGG | 5′ UTR | 5′-GCCGCCGCCGCCGCCGCCGCC-3′ |
| ovarian insufficiency | | | | (SEQ ID NO: 77) |
| Spinocerebellar ataxia type 36 | NOP56 | GGCCTG | Intron | 5′-GCCCAGGCCCAGGCCCAGGCC-3′ |
| | | | | (SEQ ID NO: 78) |
| Spinocerebellar ataxia type 31 | BEAN1/ | TGGAA/ | Intron | 5′-GTTAGGTTAGGTTAGGTTAGG-3′ |
| | TK2b | TTCCA | | (SEQ ID NO: 79) |
| Spinocerebellar ataxia type 10 | ATXN10 | ATTCT | Intron | 5′-GAATAGAATAGAATAGAATAG-3′ |
| | | | | (SEQ ID NO: 80) |

TABLE 3
Repeat expansion diseases

| Disease | Host gene | Repeat motif | Location |
|---|---|---|---|
| Unverricht-Lundborg disease | CSTB | CCCCGCCCCGCG | Promoter |
| | | (SEQ ID NO: 81) | |
| Baratela-Scott syndrome | XYLT1 | CGG | Promoter, |
| | | | 5′ UTR |
| Glutaminase deficiency | GLS | CAG | 5′ UTR |
| Spinocerebellar ataxia type 12 | PPP2R2B | CAG | 5′ UTR |
| Fragile XE syndrome | AFF2 | CCG | 5′ UTR |
| Jacobsen syndrome | CBL2 | CCG | 5′ UTR |
| Intellectual disability associated | AFF3 | CGG | 5′ UTR |
| with fragile site FRA2A | | | |
| Intellectual disability associated | DIP2B | CGG | 5′ UTR |
| with fragile site FRA12A | | | |
| Fragile X syndrome | FMR1 | CGG | 5′ UTR |
| Fragile X-associated primary | FMR1 | CGG | 5′ UTR |
| ovarian insufficiency | | | |
| Fragile X-associated tremor/ataxia | FMR1 | CGG | 5′ UTR |
| syndrome | | | |
| Neuronal intranuclear inclusion | NOTCH2NLC | CGG | 5′ UTR |
| disease | | | |
| Oculopharyngodistal myopathy 1 | LRP12 | CGG | 5′ UTR |
| Oculopharyngodistal myopathy 2 | GIPC1 | CGG | 5′ UTR |
| Oculopharyngeal myopathy with | LOC642361 and | CGG/CCG | lncRNA |
| leukoencephalopathy | NUTM2B-AS1b | | |
| Cerebellar ataxia, neuropathy and | RFC1 | AAGGG | Intron |
| vestibular areflexia syndrome | | | |
| Spinocerebellar ataxia type 10 | ATXN10 | ATTCT | Intron |
| X-linked dystonia parkinsonism | TAF1 | CCCTCT | Intron |
| Myotonic dystrophy type 2 | CNBP | CCTG | Intron |
| Autism spectrum disorder | ZNF713 | CGG | Intron |
| associated with fragile site | | | |
| FRA7A | | | |
| Fuchs endothelial corneal | TCF4 | CTG | Intron |
| dystrophy | | | |
| Friedreich ataxia | FXN | GAA | Intron |
| Spinocerebellar ataxia type 36 | NOP56 | GGCCTG | Intron |
| C9orf72 amyotrophic lateral | C9orf72 | GGGGCC | Intron |
| sclerosis and/or frontotemporal | | | |
| dementia | | | |
| Spinocerebellar ataxia type 31 | BEAN1/TK2b | TGGAA/TTCCA | Intron |
| Familial adult myoclonic epilepsy | SAMD12 | TTTCA | Intron |
| 1 | | | |
| Familial adult myoclonic epilepsy | STARD7 | TTTCA | Intron |
| 2 | | | |
| Familial adult myoclonic epilepsy | Mar-06 | TTTCA | Intron |
| 3 | | | |
| Familial adult myoclonic epilepsy | YEATS2 | TTTCA | Intron |
| 4 | | | |
| Familial adult myoclonic epilepsy | TNRC6A | TTTCA | Intron |
| 6 | | | |
| Familial adult myoclonic epilepsy | RAPGEF2 | TTTCA | Intron |
| 7 | | | |
| Spinocerebellar ataxia type 37 | DAB1 | TTTCA | Intron |
| Dentatorubropallidoluysian | ATN1 | CAG | CDS |
| atrophy | | | |
| Huntington disease | HTT | CAG | CDS |
| Spinal and bulbar muscular | AR | CAG | CDS |
| atrophy | | | |
| Spinocerebellar ataxia type 1 | ATXN1 | CAG | CDS |
| Spinocerebellar ataxia type 2 | ATXN2 | CAG | CDS |
| Spinocerebellar ataxia type 3 | ATXN3 | CAG | CDS |
| Spinocerebellar ataxia type 6 | CACNA1A | CAG | CDS |
| Spinocerebellar ataxia type 7 | ATXN7 | CAG | CDS |
| Spinocerebellar ataxia type 17 | TBP | CAG | CDS |
| Pseudoachondroplasia and | COMP | GAC | CDS |
| multiple epiphyseal dysplasia | | | |
| Blepharophimosis, ptosis and | FOXL2 | GCN | CDS |
| epicanthus inversus syndrome | | | |
| Cleidocranial dysplasia | RUNX2 | GCN | CDS |
| Congenital central | PHOX2B | GCN | CDS |
| hypoventilation syndrome | | | |
| Hand-foot-genital syndrome | HOXA13 | GCN | CDS |
| Holoprosencephaly 5 | ZIC2 | GCN | CDS |
| Oculopharyngeal muscular | PABPN1 | GCN | CDS |
| dystrophy | | | |
| Synpolydactyly 1 | HOXD13 | GCN | CDS |
| X-linked hypopituitarism | SOX3 | GCN | CDS |
| X-linked intellectual disability | ARX | GCN | CDS |
| Spinocerebellar ataxia type 8 | ATXN8OS and | CTG/CAG | 3′ UTR and |
| | ATXN8b | | CDS |
| Myotonic dystrophy type 1 | DMPK | CTG | 3′ UTR |
| Huntington disease-like 2 | JPH3 | CTG | 3′ UTR |

1. A method of modulating the function of a regulatory element in a target RNA, comprising delivering to a cell a catalytically inactive Cas13 protein (dCas13) and a CRISPR RNA (crRNA), wherein the crRNA recruits the dCas13 protein to the regulatory element, such that the function of the regulatory element is modulated.

2. The method of claim 1, wherein the Cas13 protein is a member of the Cas13b family, Cas13a family, Cas13d family, Cas13X family, Cas13Y family, or Cas13bt family.

3. The method of claim 2, wherein the Cas13 protein is:
(a) PspCas13b, RfxCas13d, LwaCas13a, Pgu13b, PgiCas13b, Cas13X.1, Cas13X.2, PinCas13b, Cas13Y.1, Cas13Y.2, Cas13Y.3, AdmCas13d, LbmCas13a, LbfCas13a, LbuCas13a, HheCas13a, Cas13bt1, or Cas13bt3;
(b) PspCas13b, RfxCas13d, LwaCas13a, PguCas13b, PgiCas13b, Cas13X.1, Cas13X.2, PinCas13b, Cas13Y.1, Cas13Y.2, AdmCas13d, LbmCas13a, LbfCas13a, LbuCas13a, HheCas13a, Cas13bt1, or Cas13bt3; or
(c) PspCas13b.

4. (canceled)

5. The method of claim 1, wherein the dCas13 protein comprises mutation in one or more amino acid residues in a RxxxxH motif of an HEPN-1 domain and a RxxxxH motif of an HEPN-2 domain relative to a corresponding unmodified Cas13 protein.

6. The method of claim 1, wherein the dCas13 protein comprises or consists of an amino acid sequence having a sequence identity of 85% to SEQ ID NO: 1, provided that the amino acid residues corresponding to the amino acid residues at positions 133 and 1058 of SEQ ID NO: 1 are not histidine.

7. The method of claim 1, wherein the dCas13 protein is truncated by ≤110 amino acid residues from the C-terminus compared to the unmodified protein, wherein the Cas13 protein of the dCas13 protein is a member of the Cas13b family.

8. The method of claim 7, wherein the dCas13 protein comprises or consists of an amino acid sequence having a sequence identity of ≥85% to SEQ ID NO: 2 or 3, provided that the amino acid residue corresponding to the amino acid residue at position 133 of SEQ ID NO: 2 or 3 is not histidine.

9. The method of claim 1, wherein the crRNA comprises a dCas13-specific direct repeat and a spacer which is capable of specifically hybridizing with the target RNA sequence.

10. The method of claim 9, wherein the crRNA comprises a dCas13b-specific direct repeat and the spacer has a length of between 12 to 36 nucleotides, 12 to 27 nucleotides, or 18 to 24 nucleotides.

11. The method of claim 1, wherein the regulatory element is: (a) a cis-regulatory element, or (b) a trans-regulatory element.

12. The method of claim 11, wherein:
(a) the protein binding site is an expanded repeat sequence, a start codon of an open reading frame, a start codon of an upstream open reading frame (uORF), or a snRNP binding site; or
(b) the nucleic acid binding site is a miRNA binding site.

13. The method of claim 1, wherein the method is for modulating the availability, expression and/or activity of a nucleic acid or protein of interest, wherein the target RNA encodes or regulates the nucleic acid or protein of interest, such that the availability, expression and/or activity of the nucleic acid or protein of interest is increased or decreased, or the method is for blocking an miRNA-binding site, wherein the crRNA comprises a spacer that is complementary to the miRNA-binding site, such that miRNA-mediated silencing of the target RNA is reduced, or the method is for blocking ribosomal attachment or translation, wherein the crRNA comprises a spacer that is complementary to a start codon of an open reading frame or a start codon of an upstream open reading frame (uORF), such that translation of the target RNA is reduced, or the method is for inducing splice switching, wherein the crRNA comprises a spacer that is complementary to a splicing element wherein splicing of the target RNA is modulated.

14-16. (canceled)

17. A crRNA specific for dCas13b comprising: (i) a dCas13b-specific direct repeat, and (ii) a spacer which is capable of specifically hybridizing with the target RNA sequence and having length of between 18 to 24 nucleotides.

18. A dCas13b protein consisting of an amino acid sequence having a sequence identity of ≥85% to SEQ ID NO: 2 or 3, provided that the amino acid residues corresponding to the amino acid residue at position 133 of SEQ ID NO: 2 or 3 is alanine.

19. A polynucleotide or vector encoding a crRNA comprising (i) a dCas13b-specific direct repeat, and (ii) a spacer which is capable of specifically hybridizing with the target RNA sequence and having length of between 18 to 24 nucleotides, or a dCas13b protein consisting of an amino acid sequence having a sequence identity of ≥85% to SEQ ID NO: 2 or 3, provided that the amino acid residues corresponding to the amino acid residue at position 133 of SEQ ID NO: 2 or 3 is alanine, optionally wherein the vector is AAV or lentivirus.

20. A delivery vehicle comprising a crRNA comprising (i) a dCas13b-specific direct repeat, and (ii) a spacer which is capable of specifically hybridizing with the target RNA sequence and having length of between 18 to 24 nucleotides, a dCas13b protein consisting of an amino acid sequence having a sequence identity of ≥85% to SEQ ID NO: 2 or 3, provided that the amino acid residues corresponding to the amino acid residue at position 133 of SEQ ID NO: 2 or 3 is alanine, or the polynucleotide or vector according to claim 19.

21. A pharmaceutical composition comprising: (i) a crRNA comprising (a) a dCas13b-specific direct repeat, and (b) a spacer which is capable of specifically hybridizing with the target RNA sequence and having length of between 18 to 24 nucleotides, or a dCas13b protein consisting of an amino acid sequence having a sequence identity of ≥85% to SEQ ID NO: 2 or 3, provided that the amino acid residues corresponding to the amino acid residue at position 133 of SEQ ID NO: 2 or 3 is alanine, (ii) and a pharmaceutically acceptable carrier.

22-23. (canceled)

24. A method of treating or preventing a repeat expansion disease in a subject, wherein the method comprises administering to a subject a therapeutically effective amount of a dCas13 protein and crRNA, wherein the method comprises delivering to a cell a catalytically inactive Cas13 protein (dCas13) and a CRISPR RNA (crRNA), wherein the crRNA recruits the dCas13 protein to the regulatory element, such that the function of the regulatory element is modulated, or modulating the availability, expression and/or activity of a nucleic acid or protein of interest according to the method of claim 13, wherein the crRNA comprises a spacer complementary to an expanded repeat sequence, and optionally wherein the expansion disease is type 1 myotonic dystrophy (DM1), myotonic dystrophy type 2 or Fuchs endothelial corneal dystrophy.

25. (canceled)

26. The method of claim 24, wherein the Cas13 protein is a member of the Cas13b family, and optionally wherein the dCas13 protein comprises or consists of an amino acid sequence having a sequence identity of ≥85% to SEQ ID NO: 2 or 3, provided that the amino acid residue corresponding to the amino acid residue at position 133 of SEQ ID NO: 2 or 3 is alanine.


SEQUENCE LISTING (82 sequences)

SEQ ID NO: 1 - protein, 1090 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMK

SEQ ID NO: 2 - protein, 985 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIM

SEQ ID NO: 3 - protein, 1054 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRN

SEQ ID NO: 4 - RNA, 46 nt, synthetic construct
  source 1..46: mol_type = other RNA
nnnnnnnnnngttgtggaaggtccagttttagggggctattacaac

SEQ ID NO: 5 - DNA, 123 bp, synthetic construct
  source 1..123: mol_type = other DNA
aaacctctacaaatgtggtatggctgattatgatccggatccaaacaccgnnnnnnnnnnnnnnnnnnnnnnngttttacctgtcagctagcactcacgtgctaccccctacacattgatcct

SEQ ID NO: 6 - DNA, 10 bp, synthetic construct
  source 1..10: mol_type = other DNA
aaaaaaaaaa

SEQ ID NO: 7 - RNA, 80 nt, synthetic construct
  source 1..80: mol_type = other RNA
ggagacgaataacgttatatcctgattcacgtaatatcgtctcagttgtggaaggtccagttttagggggctattacaac

SEQ ID NO: 8 - RNA, 59 nt, synthetic construct
  source 1..59: mol_type = other RNA
gagcagcagcagcagcagcagcagttgtggaaggtccagttttagggggctattacaac

SEQ ID NO: 9 - RNA, 67 nt, synthetic construct
  source 1..67: mol_type = other RNA
gagcagcagcagcagcagcagcagcagcagcgttgtggaaggtccagttttagggggctattacaac

SEQ ID NO: 10 - RNA, 21 nt, synthetic construct
  source 1..21: mol_type = other RNA
gcagcagcagcagcagcagca

SEQ ID NO: 11 - RNA, 60 nt, synthetic construct
  source 1..60: mol_type = other RNA
gcagcagcagcagcagcagcagcagttgtggaaggtccagttttagggggctattacaac

SEQ ID NO: 12 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 13 - protein, 1152 aa, Leptotrichia wadei
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 14 - protein, 1120 aa, Listeria seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 15 - protein, 1340 aa, Lachnospira sp.
MQISKVNHKHVAVGQKDRERITGFIYNDPVGDEKSLEDVVAKRANDTKVLFNVFNTKDLYDSQESDKSEKDKEIISKGAKFVAKSFNSAITILKKQNKIYSTLTSQQVIKELKDKFGGARIYDDDIEEALTETLKKSFRKENVRNSIKVLIENAAGIRSSLSKDEEELIQEYFVKQLVEEYTKTKLQKNVVKSIKNQNMVIQPDSDSQVLSLSESRREKQSSAVSSDTLVNCKEKDVLKAFLTDYAVLDEDERNSLLWKLRNLVNLYFYGSESIRDYSYTKEKSVWKEHDEQKANKTLFIDEICHITKIGKNGKEQKVLDYEENRSRCRKQNINYYRSALNYAKNNTSGIFENEDSNHFWIHLIENEVERLYNGIENGEEFKFETGYISEKVWKAVINHLSIKYIALGKAVYNYAMKELSSPGDIEPGKIDDSYINGITSFDYEIIKAEESLQRDISMNVVFATNYLACATVDTDKDFLLFSKEDIRSCTKKDGNLCKNIMQFWGGYSTWKNFCEEYLKDDKDALELLYSLKSMLYSMRNSSFHFSTENVDNGSWDTELIGKLFEEDCNRAARIEKEKFYNNNLHMFYSSSLLEKVLERLYSSHHERASQVPSFNRVFVRKNFPSSLSEQRITPKFTDSKDEQIWQSAVYYLCKEIYYNDFLQSKEAYKLFREGVKNLDKNDINNQKAADSFKQAVVYYGKAIGNATLSQVCQAIMTEYNRQNNDGLKKKSAYAEKQNSNKYKHYPLFLKQVLQSAFWEYLDENKEIYGFISAQIHKSNVEIKAEDFIANYSSQQYKKLVDKVKKTPELQKWYTLGRLINPRQANQFLGSIRNYVQFVKDIQRRAKENGNPIRNYYEVLESDSIIKILEMCTKLNGTTSNDIHDYFRDEDEYAEYISQFVNFGDVHSGAALNAFCNSESEGKKNGIYYDGINPIVNRNWVLCKLYGSPDLISKIISRVNENMIHDFHKQEDLIREYQIKGICSNKKEQQDLRTFQVLKNRVELRDIVEYSEIINELYGQLIKWCYLRERDLMYFQLGFHYLCLNNASSKEADYIKINVDDRNISGAILYQIAAMYINGLPVYYKKDDMYVALKSGKKASDELNSNEQTSKKINYFLKYGNNILGDKKDQLYLAGLELFENVAEHENIIIFRNEIDHFHYFYDRDRSMLDLYSEVFDRFFTYDMKLRKNVVNMLYNILLDHNIVSSFVFETGEKKVGRGDSEVIKPSAKIRLRANNGVSSDVFTYKVGSKDELKIATLPAKNEEFLLNVARLIYYPDMEAVSENMVREGVVKVEKSNDKKGKISRGSNTRSSNQSKYNNKSKNRMNYSMGSIFEKMDLKFD

SEQ ID NO: 16 - protein, 1437 aa, Lachnospira sp.
MKISKVREENRGAKLTVNAKTAVVSENRSQEGILYNDPSRYGKSRKNDEDRDRYIESRLKSSGKLYRIFNEDKNKRETDELQWFLSEIVKKINRRNGLVLSDMLSVDDRAFEKAFEKYAELSYTNRRNKVSGSPAFETCGVDAATAERLKGIISETNFINRIKNNIDNKVSEDIIDRIIAKYLKKSLCRERVKRGLKKLLMNAFDLPYSDPDIDVQRDFIDYVLEDFYHVRAKSQVSRSIKNMNMPVQPEGDGKFAITVSKGGTESGNKRSAEKEAFKKFLSDYASLDERVRDDMLRRMRRLVVLYFYGSDDSKLSDVNEKFDVWEDHAARRVDNREFIKLPLENKLANGKTDKDAERIRKNTVKELYRNQNIGCYRQAVKAVEEDNNGRYFDDKMLNMFFIHRIEYGVEKIYANLKQVTEFKARTGYLSEKIWKDLINYISIKYIAMGKAVYNYAMDELNASDKKEIELGKISEEYLSGISSFDYELIKAEEMLQRETAVYVAFAARHLSSQTVELDSENSDFLLLKPKGTMDKNDKNKLASNNILNFLKDKETLRDTILQYFGGHSLWTDFPFDKYLAGGKDDVDFLTDLKDVIYSMRNDSFHYATENHNNGKWNKELISAMFEHETERMTVVMKDKFYSNNLPMFYKNDDLKKLLIDLYKDNVERASQVPSFNKVFVRKNFPALVRDKDNLGIELDLKADADKGENELKFYNALYYMFKEIYYNAFLNDKNVRERFITKATKVADNYDRNKERNLKDRIKSAGSDEKKKLREQLQNYIAENDFGQRIKNIVQVNPDYTLAQICQLIMTEYNQQNNGCMQKKSAARKDINKDSYQHYKMLLLVNLRKAFLEFIKENYAFVLKPYKHDLCDKADFVPDFAKYVKPYAGLISRVAGSSELQKWYIVSRFLSPAQANHMLGFLHSYKQYVWDIYRRASETGTEINHSIAEDKIAGVDITDVDAVIDLSVKLCGTISSEISDYFKDDEVYAEYISSYLDFEYDGGNYKDSLNRFCNSDAVNDQKVALYYDGEHPKLNRNIILSKLYGERRFLEKITDRVSRSDIVEYYKLKKETSQYQTKGIFDSEDEQKNIKKFQEMKNIVEFRDLMDYSEIADELQGQLINWIYLRERDLMNFQLGYHYACLNNDSNKQATYVTLDYQGKKNRKINGAILYQICAMYINGLPLYYVDKDSSEWTVSDGKESTGAKIGEFYRYAKSFENTSDCYASGLEIFENISEHDNITELRNYIEHFRYYSSFDRSFLGIYSEVFDRFFTYDLKYRKNVPTILYNILLQHFVNVRFEFVSGKKMIGIDKKDRKIAKEKECARITIREKNGVYSEQFTYKLKNGTVYVDARDKRYLQSIIRLLFYPEKVNMDEMIEVKEKKKPSDNNTGKGYSKRDRQQDRKEYDKYKEKKKKEGNFLSGMGGNINWDEINAQLKN

SEQ ID NO: 17 - protein, 1385 aa, Clostridium aminophilum
MKFSKVDHTRSAVGIQKATDSVHGMLYTDPKKQEVNDLDKRFDQLNVKAKRLYNVFNQSKAEEDDDEKRFGKVVKKLNRELKDLLFHREVSRYNSIGNAKYNYYGIKSNPEEIVSNLGMVESLKGERDPQKVISKLLLYYLRKGLKPGTDGLRMILEASCGLRKLSGDEKELKVFLQTLDEDFEKKTFKKNLIRSIENQNMAVQPSNEGDPIIGITQGRFNSQKNEEKSAIERMMSMYADLNEDHREDVLRKLRRLNVLYFNVDTEKTEEPTLPGEVDTNPVFEVWHDHEKGKENDRQFATFAKILTEDRETRKKEKLAVKEALNDLKSAIRDHNIMAYRCSIKVTEQDKDGLFFEDQRINRFWIHHIESAVERILASINPEKLYKLRIGYLGEKVWKDLLNYLSIKYIAVGKAVFHFAMEDLGKTGQDIELGKLSNSVSGGLTSFDYEQIRADETLQRQLSVEVAFAANNLFRAVVGQTGKKIEQSKSEENEEDFLLWKAEKIAESIKKEGEGNTLKSILQFFGGASSWDLNHFCAAYGNESSALGYETKFADDLRKAIYSLRNETFHFTTLNKGSFDWNAKLIGDMFSHEAATGIAVERTRFYSNNLPMFYRESDLKRIMDHLYNTYHPRASQVPSFNSVFVRKNFRLFLSNTLNTNTSFDTEVYQKWESGVYYLFKEIYYNSFLPSGDAHHLFFEGLRRIRKEADNLPIVGKEAKKRNAVQDFGRRCDELKNLSLSAICQMIMTEYNEQNNGNRKVKSTREDKRKPDIFQHYKMLLLRTLQEAFAIYIRREEFKFIFDLPKTLYVMKPVEEFLPNWKSGMFDSLVERVKQSPDLQRWYVLCKFLNGRLLNQLSGVIRSYIQFAGDIQRRAKANHNRLYMDNTQRVEYYSNVLEVVDFCIKGTSRFSNVFSDYFRDEDAYADYLDNYLQFKDEKIAEVSSFAALKTFCNEEEVKAGIYMDGENPVMQRNIVMAKLFGPDEVLKNVVPKVTREEIEEYYQLEKQIAPYRQNGYCKSEEDQKKLLRFQRIKNRVEFQTITEFSEIINELLGQLISWSFLRERDLLYFQLGFHYLCLHNDTEKPAEYKEISREDGTVIRNAILHQVAAMYVGGLPVYTLADKKLAAFEKGEADCKLSISKDTAGAGKKIKDFFRYSKYVLIKDRMLTDQNQKYTIYLAGLELFENTDEHDNITDVRKYVDHFKYYATSDENAMSILDLYSEIHDRFFTYDMKYQKNVANMLENILLRHFVLIRPEFFTGSKKVGEGKKITCKARAQIEIAENGMRSEDFTYKLSDGKKNISTCMIAARDQKYLNTVARLLYYPHEAKKSIVDTREKKNNKKTNRGDGTFNKQKGTARKEKDNGPREFNDTGFSNTPFAGFDPFRNS

SEQ ID NO: 18 - protein, 1175 aa, Carnobacterium gallinarum
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHGMILKLKIDENHKNFEIESLIPKEIIHLKDKAIKTNQVSEEYCQLVLALLTTNPGNQLN

SEQ ID NO: 19 - protein, 1164 aa, Carnobacterium gallinarum
MRMTKVKINGSPVSMNRSKLNGHLVWNGTTNTVNILTKKEQSFAASFLNKTLVKADQVKGYKVLAENIFIIFEQLEKSNSEKPSVYLNNIRRLKEAGLKRFFKSKYHEEIKYTSEKNQSVPTKLNLIPLFFNAVDRIQEDKFDEKNWSYFCKEMSPYLDYKKSYLNRKKEILANSIQQNRGFSMPTAEEPNLLSKRKQLFQQWAMKFQESPLIQQNNFAVEQFNKEFANKINELAAVYNVDELCTAITEKLMNFDKDKSNKTRNFEIKKLWKQHPHNKDKALIKLFNQEGNEALNQFNIELGKYFEHYFPKTGKKESAESYYLNPQTIIKTVGYQLRNAFVQYLLQVGKLHQYNKGVLDSQTLQEIGMYEGFQTKFMDACVFASSSLRNIIQATTNEDILTREKFKKELEKNVELKHDLFFKTEIVEERDENPAKKIAMTPNELDLWAIRGAVQRVRNQIFHQQINKRHEPNQLKVGSFENGDLGNVSYQKTIYQKLFDAEIKDIEIYFAEKIKSSGALEQYSMKDLEKLFSNKELTLSLGGQVVAFAPSYKKLYKQGYFYQNEKTIELEQFTDYDFSNDVFKANYYLIKLIYHYVFLPQFSQANNKLFKDTVHYVIQQNKELNTTEKDKKNNKKIRKYAFEQVKLMKNESPEKYMQYLQREMQEERTIKEAKKTNEEKPNYNFEKLLIQIFIKGFDTFLRNFDLNLNPAEELVGTVKEKAEGLRKRKERIAKILNVDEQIKTGDEEIAFWIFAKLLDARHLSELRNEMIKFKQSSVKKGLIKNGDLIEQMQPILELCILSNDSESMEKESFDKIEVFLEKVELAKNEPYMQEDKLTPVKFRFMKQLEKYQTRNFIENLVIENPEFKVSEKIVLNWHEEKEKIADLVDKRTKLHEEWASKAREIEEYNEKIKKNKSKKLDKPAEFAKFAEYKIICEAIENFNRLDHKVRLTYLKNLHYLMIDLMGRMVGFSVLFERDFVYMGRSYSALKKQSIYLNDYDTFANIRDWEVNENKHLFGTSSSDLTFQETAEFKNLKKPMENQLKALLGVTNHSFEIRNNIAHLHVLRNDGKGEGVSLLSCMNDLRKLMSYDRKLKNAVTKAIIKILDKHGMILKLTNNDHTKPFEIESLKPKKIIHLEKSNHSFPMDQVSQEYCDLVKKMLVFTN

SEQ ID NO: 20 - protein, 1154 aa, Paludibacter propionicigenes
MRVSKVKVKDGGKDKMVLVHRKTTGAQLVYSGQPVSNETSNILPEKKRQSFDLSTLNKTIIKFDTAKKQKLNVDQYKIVEKIFKYPKQELPKQIKAEEILPFLNHKFQEPVKYWKNGKEESFNLTLLIVEAVQAQDKRKLQPYYDWKTWYIQTKSDLLKKSIENNRIDLTENLSKRKKALLAWETEFTASGSIDLTHYHKVYMTDVLCKMLQDVKPLTDDKGKINTNAYHRGLKKALQNHQPAIFGTREVPNEANRADNQLSIYHLEVVKYLEHYFPIKTSKRRNTADDIAHYLKAQTLKTTIEKQLVNAIRANIIQQGKTNHHELKADTTSNDLIRIKTNEAFVLNLTGTCAFAANNIRNMVDNEQTNDILGKGDFIKSLLKDNTNSQLYSFFFGEGLSTNKAEKETQLWGIRGAVQQIRNNVNHYKKDALKTVFNISNFENPTITDPKQQTNYADTIYKARFINELEKIPEAFAQQLKTGGAVSYYTIENLKSLLTTFQFSLCRSTIPFAPGFKKVFNGGINYQNAKQDESFYELMLEQYLRKENFAEESYNARYFMLKLIYNNLFLPGFTTDRKAFADSVGFVQMQNKKQAEKVNPRKKEAYAFEAVRPMTAADSIADYMAYVQSELMQEQNKKEEKVAEETRINFEKFVLQVFIKGFDSFLRAKEFDFVQMPQPQLTATASNQQKADKLNQLEASITADCKLTPQYAKADDATHIAFYVFCKLLDAAHLSNLRNELIKFRESVNEFKFHHLLEIIEICLLSADVVPTDYRDLYSSEADCLARLRPFIEQGADITNWSDLFVQSDKHSPVIHANIELSVKYGTTKLLEQIINKDTQFKTTEANFTAWNTAQKSIEQLIKQREDHHEQWVKAKNADDKEKQERKREKSNFAQKFIEKHGDDYLDICDYINTYNWLDNKMHFVHLNRLHGLTIELLGRMAGFVALFDRDFQFFDEQQIADEFKLHGFVNLHSIDKKLNEVPTKKIKEIYDIRNKIIQINGNKINESVRANLIQFISSKRNYYNNAFLHVSNDEIKEKQMYDIRNHIAHFNYLTKDAADFSLIDLINELRELLHYDRKLKNAVSKAFIDLFDKHGMILKLKLNADHKLKVESLEPKKIYHLGSSAKDKPEYQYCTNQVMMAYCNMCRSLLEMKK

SEQ ID NO: 21 - protein, 970 aa, Listeria weihenstephanensis
MLALLHQEVPSQKLHNLKSLNTESLTKLFKPKFQNMISYPPSKGAEHVQFCLTDIAVPAIRDLDEIKPDWGIFFEKLKPYTDWAESYIHYKQTTIQKSIEQNKIQSPDSPRKLVLQKYVTAFLNGEPLGLDLVAKKYKLADLAESFKVVDLNEDKSANYKIKACLQQHQRNILDELKEDPELNQYGIEVKKYIQRYFPIKRAPNRSKHARADFLKKELIESTVEQQFKNAVYHYVLEQGKMEAYELTDPKTKDLQDIRSGEAFSFKFINACAFASNNLKMILNPECEKDILGKGDFKKNLPNSTTQSDVVKKMIPFFSDEIQNVNFDEAIWAIRGSIQQIRNEVYHCKKHSWKSILKIKGFEFEPNNMKYTDSDMQKLMDKDIAKIPDFIEEKLKSSGIIRFYSHDKLQSIWEMKQGFSLLTTNAPFVPSFKRVYAKGHDYQTSKNRYYDLGLTTFDILEYGEEDFRARYFLTKLVYYQQFMPWFTADNNAFRDAANFVLRLNKNRQQDAKAFINIREVEEGEMPRDYMGYVQGQIAIHEDSTEDTPNHFEKFISQVFIKGFDSHMRSADLKFIKNPRNQGLEQSEIEEMSFDIKVEPSFLKNKDDYIAFWTFCKMLDARHLSELRNEMIKYDGHLTGEQEIIGLALLGVDSRENDWKQFFSSEREYEKIMKGYVGEELYQREPYRQSDGKTPILFRGVEQARKYGTETVIQRLFDASPEFKVSKCNITEWERQKETIEETIERRKELHNEWEKNPKKPQNNAFFKEYKECCDAIDAYNWHKNKTTLVYVNELHHLLIEILGRYVGYVAIADRDFQCMANQYFKHSGITERVEYWGDNRLKSIKKLDTFLKKEGLFVSEKNARNHIAHLNYLSLKSECTLLYLSERLREIFKYDRKLKNAVSKSLIDILDRHGMSVVFANLKENKHRLVIKSLEPKKLRHLGEKKIDNGYIETNQVSEEYCGIVKRLLEI

SEQ ID NO: 22 - protein, 1152 aa, Leptotrichia wadei; identical to SEQ ID NO: 13

SEQ ID NO: 23 - protein, 1051 aa, Listeria newyorkensis
MKITKMRVDGRTIVMERTSKEGQLGYEGIDGNKTTEIIFDKKKESFYKSILNKTVRKPDEKEKNRRKQAINKAINKEITELMLAVLHQEVPSQKLHNLKSLNTESLTKLFKPKFQNMISYPPSKGAEHVQFCLTDIAVPAIRDLDEIKPDWGIFFEKLKPYTDWAESYIHYKQTTIQKSIEQNKIQSPDSPRKLVLQKYVTAFLNGEPLGLDLVAKKYKLADLAESFKLVDLNEDKSANYKIKACLQQHQRNILDELKEDPELNQYGIEVKKYIQRYFPIKRAPNRSKHARADFLKKELIESTVEQQFKNAVYHYVLEQGKMEAYELTDPKTKDLQDIRSGEAFSFKFINACAFASNNLKMILNPECEKDILGKGNFKKNLPNSTTRSDVVKKMIPFFSDELQNVNFDEAIWAIRGSIQQIRNEVYHCKKHSWKSILKIKGFEFEPNNMKYADSDMQKLMDKDIAKIPEFIEEKLKSSGVVRFYRHDELQSIWEMKQGFSLLTTNAPFVPSFKRVYAKGHDYQTSKNRYYNLDLTTFDILEYGEEDFRARYFLTKLVYYQQFMPWFTADNNAFRDAANFVLRLNKNRQQDAKAFINIREVEEGEMPRDYMGYVQGQIAIHEDSIEDTPNHFEKFISQVFIKGFDRHMRSANLKFIKNPRNQGLEQSEIEEMSFDIKVEPSFLKNKDDYIAFWIFCKMLDARHLSELRNEMIKYDGHLTGEQEIIGLALLGVDSRENDWKQFFSSEREYEKIMKGYVVEELYQREPYRQSDGKTPILFRGVEQARKYGTETVIQRLFDANPEFKVSKCNLAEWERQKETIEETIKRRKELHNEWAKNPKKPQNNAFFKEYKECCDAIDAYNWHKNKTTLAYVNELHHLLIEILGRYVGYVAIADRDFQCMANQYFKHSGITERVEYWGDNRLKSIKKLDTFLKKEGLFVSEKNARNHIAHLNYLSLKSECTLLYLSERLREIFKYDRKLKNAVSKSLIDILDRHGMSVVFANLKENKHRLVIKSLEPKKLRHLGGKKIDGGYIETNQVSEEYCGIVKRLLEM

SEQ ID NO: 24 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRTSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 25 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 26 - protein, 1285 aa, Rhodobacter capsulatus; identical to SEQ ID NO: 25

SEQ ID NO: 27 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 28 - protein, 1285 aa, Herbinix hemicellulosilytica
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 29 - protein, 1344 aa, Eubacterium rectale
MLRRDKEVKKLYNVFNQIQVGTKPKKWNNDEKLSPEENERRAQQKNIKMKNYKWREACSKYVESSQRIINDVIFYSYRKAKNKLRYMRKNEDILKKMQEAEKLSKFSGGKLEDFVAYTLRKSLVVSKYDTQEFDSLAAMVVFLECIGKNNISDHEREIVCKLLELIRKDFSKLDPNVKGSQGANIVRSVRNQNMIVQPQGDRFLFPQVYAKENETVTNKNVEKEGLNEFLLNYANLDDEKRAESLRKLRRILDVYFSAPNHYEKDMDITLSDNIEKEKFNVWEKHECGKKETGLFVDIPDVLMEAEAENIKLDAVVEKRERKVLNDRVRKQNIICYRYTRAVVEKYNSNEPLFFENNAINQYWIHHIENAVERILKNCKAGKLFKLRKGYLAEKVWKDAINLISIKYIALGKAVYNFALDDIWKDKKNKELGIVDERIRNGITSFDYEMIKAHENLQRELAVDIAFSVNNLARAVCDMSNLGNKESDFLLWKRNDIADKLKNKDDMASVSAVLQFFGGKSSWDINIFKDAYKGKKKYNYEVRFIDDLRKAIYCARNENFHFKTALVNDEKWNTELFGKIFERETEFCLNVEKDRFYSNNLYMFYQVSELRNMLDHLYSRSVSRAAQVPSYNSVIVRTAFPEYITNVLGYQKPSYDADTLGKWYSACYYLLKEIYYNSFLQSDRALQLFEKSVKTLSWDDKKQQRAVDNFKDHFSDIKSACTSLAQVCQIYMTEYNQQNNQIKKVRSSNDSIFDQPVYQHYKVLLKKAIANAFADYLKNNKDLFGFIGKPFKANEIREIDKEQFLPDWTSRKYEALCIEVSGSQELQKWYIVGKFLNARSLNLMVGSMRSYIQYVTDIKRRAASIGNELHVSVHDVEKVEKWVQVIEVCSLLASRTSNQFEDYFNDKDDYARYLKSYVDFSNVDMPSEYSALVDFSNEEQSDLYVDPKNPKVNRNIVHSKLFAADHILRDIVEPVSKDNIEEFYSQKAEIAYCKIKGKEITAEEQKAVLKYQKLKNRVELRDIVEYGEIINELLGQLINWSFMRERDLLYFQLGFHYDCLRNDSKKPEGYKNIKVDENSIKDAILYQIIGMYVNGVTVYAPEKDGDKLKEQCVKGGVGVKVSAFHRYSKYLGLNEKTLYNAGLEIFEVVAEHEDIINLRNGIDHFKYYLGDYRSMLSIYSEVFDRFFTYDIKYQKNVLNLLQNILLRHNVIVEPILESGFKTIGEQTKPGAKLSIRSIKSDTFQYKVKGGTLITDAKDERYLETIRKILYYAENEEDNLKKSVVVTNADKYEKNKESDDQNKQKEKKNKDNKGKKNEETKSDAEKNNNERLSYNPFANLNFKLSN

SEQ ID NO: 30 - protein, 1373 aa, Eubacterium sp.
MKISKESHKRTAVAVMEDRVGGVVYVPGGSGIDLSNNLKKRSMDTKSLYNVFNQIQAGTAPSEYEWKDYLSEAENKKREAQKMIQKANYELRRECEDYAKKANLAVSRIIFSKKPKKIFSDDDIISHMKKQRLSKFKGRMEDFVLIALRKSLVVSTYNQEVFDSRKAATVFLKNIGKKNISADDERQIKQLMALIREDYDKWNPDKDSSDKKESSGTKVIRSIEHQNMVIQPEKNKLSLSKISNVGKKTKTKQKEKAGLDAFLKEYAQIDENSRMEYLKKLRRLLDTYFAAPSSYIKGAAVSLPENINFSSELNVWERHEAAKKVNINFVEIPESLLNAEQNNNKINKVEQEHSLEQLRTDIRRRNITCYHFANALAADERYHTLFFENMAMNQFWIHHMENAVERILKKCNVGTLFKLRIGYLSEKVWKDMLNLLSIKYIALGKAVYHFALDDIWKADIWKDASDKNSGKINDLTLKGISSFDYEMVKAQEDLQREMAVGVAFSTNNLARVTCKMDDLSDAESDFLLWNKEAIRRHVKYTEKGEILSAILQFFGGRSLWDESLFEKAYSDSNYELKFLDDLKRAIYAARNETFHFKTAAIDGGSWNTRLFGSLFEKEAGLCLNVEKNKFYSNNLVLFYKQEDLRVFLDKLYGKECSRAAQIPSYNTILPRKSFSDFMKQLLGLKEPVYGSAILDQWYSACYYLFKEVYYNLFLQDSSAKALFEKAVKALKGADKKQEKAVESFRKRYWEISKNASLAEICQSYITEYNQQNNKERKVRSANDGMFNEPIYQHYKMLLKEALKMAFASYIKNDKELKFVYKPTEKLFEVSQDNFLPNWNSEKYNTLISEVKNSPDLQKWYIVGKFMNARMLNLLLGSMRSYLQYVSDIQKRAAGLGENQLHLSAENVGQVKKWIQVLEVCLLLSVRISDKFTDYFKDEEEYASYLKEYVDFEDSAMPSDYSALLAFSNEGKIDLYVDASNPKVNRNIIQAKLYAPDMVLKKVVKKISQDECKEFNEKKEQIMQFKNKGDEVSWEEQQKILEYQKLKNRVELRDLSEYGELINELLGQLINWSYLRERDLLYFQLGFHYSCLMNESKKPDAYKTIRRGTVSIENAVLYQIIAMYINGFPVYAPEKGELKPQCKTGSAGQKIRAFCQWASMVEKKKYELYNAGLELFEVVKEHDNIIDLRNKIDHFKYYQGNDSILALYGEIFDRFFTYDMKYRNNVLNHLQNILLRHNVIIKPIISKDKKEVGRGKMKDRAAFLLEEVSSDRFTYKVKEGERKIDAKNRLYLETVRDILYFPNRAVNDKGEDVIICSKKAQDLNEKKADRDKNHDKSKDTNQKKEGKNQEEKSENKEPYSDRMTWKPFAGIKLE

SEQ ID NO: 31 - protein, 1276 aa, Blautia sp.
MKISKVDHVKSGIDQKLSSQRGMLYKQPQKKYEGKQLEEHVRNLSRKAKALYQVFPVSGNSKMEKELQIINSFIKNILLRLDSGKTSEEIVGYINTYSVASQISGDHIQELVDQHLKESLRKYTCVGDKRIYVPDIIVALLKSKFNSETLQYDNSELKILIDFIREDYLKEKQIKQIVHSIENNSTPLRIAEINGQKRLIPANVDNPKKSYIFEFLKEYAQSDPKGQESLLQHMRYLILLYLYGPDKITDDYCEEIEAWNFGSIVMDNEQLFSEEASMLIQDRIYVNQQIEEGRQSKDTAKVKKNKSKYRMLGDKIEHSINESVVKHYQEACKAVEEKDIPWIKYISDHVMSVYSSKNRVDLDKLSLPYLAKNTWNTWISFIAMKYVDMGKGVYHFAMSDVDKVGKQDNLIIGQIDPKFSDGISSFDYERIKAEDDLHRSMSGYIAFAVNNFARAICSDEFRKKNRKEDVLTVGLDEIPLYDNVKRKLLQYFGGASNWDDSIIDIIDDKDLVACIKENLYVARNVNFHFAGSEKVQKKQDDILEEIVRKETRDIGKHYRKVFYSNNVAVFYCDEDIIKLMNHLYQREKPYQAQIPSYNKVISKTYLPDLIFMLLKGKNRTKISDPSIMNMFRGTFYFLLKEIYYNDFLQASNLKEMFCEGLKNNVKNKKSEKPYQNFMRRFEELENMGMDFGEICQQIMTDYEQQNKQKKKTATAVMSEKDKKIRTLDNDTQKYKHFRTLLYIGLREAFIIYLKDEKNKEWYEFLREPVKREQPEEKEFVNKWKLNQYSDCSELILKDSLAAAWYVVAHFINQAQLNHLIGDIKNYIQFISDIDRRAKSTGNPVSESTEIQIERYRKILRVLEFAKFFCGQITNVLTDYYQDENDFSTHVGHYVKFEKKNMEPAHALQAFSNSLYACGKEKKKAGFYYDGMNPIVNRNITLASMYGNKKLLENAMNPVTEQDIRKYYSLMAELDSVLKNGAVCKSEDEQKNLRHFQNLKNRIELVDVLTLSELVNDLVAQLIGWVYIRERDMMYLQLGLHYIKLYFTDSVAEDSYLRTLDLEEGSIADGAVLYQIASLYSFNLPMYVKPNKSSVYCKKHVNSVATKFDIFEKEYCNGDETVIENGLRLFENINLHKDMVKFRDYLAHFKYFAKLDESILELYSKAYDFFFSYNIKLKKSVSYVLTNVLLSYFINAKLSFSTYKSSGNKTVQHRTTKISVVAQTDYFTYKLRSIVKNKNGVESIENDDRRCEVVNIAARDKEFVDEVCNVINYNSDK

SEQ ID NO: 32 - protein, 1385 aa, Leptotrichia sp.
MGNLFGHKRWYEVRDKKDFKIKRKVKVKRNYDGNKYILNINENNNKEKIDNNKFIGEFVNYKKNNNVLKEFKRKFHAGNILFKLKGKEEIIRIENNDDFLETEEVVLYIEVYGKSEKLKALEITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDNKIDVILTNFMEIREKIKSNLEIMGFVKFYLNVSGDKKKSENKKMFVEKILNTNVDLTVEDIVDFIVKELKFWNITKRIEKVKKFNNEFLENRRNRTYIKSYVLLDKHEKFKIERENKKDKIVKFFVENIKNNSIKEKIEKILAEFKINELIKKLEKELKKGNCDTEIFGIFKKHYKVNFDSKKFSNKSDEEKELYKIIYRYLKGRIEKILVNEQKVRLKKMEKIEIEKILNESILSEKILKRVKQYTLEHIMYLGKLRHNDIVKMTVNTDDFSRLHAKEELDLELITFFASTNMELNKIFNGKEKVTDFFGFNLNGQKITLKEKVPSFKLNILKKLNFINNENNIDEKLSHFYSFQKEGYLLRNKILHNSYGNIQETKNLKGEYENVEKLIKELKVSDEEISKSLSLDVIFEGKVDIINKINSLKIGEYKDKKYLPSFSKIVLEITRKFREINKDKLFDIESEKIILNAVKYVNKILYEKITSNEENEFLKTLPDKLVKKSNNKKENKNLLSIEEYYKNAQVSSSKGDKKAIKKYQNKVTNAYLEYLENTFTEIIDFSKFNLNYDEIKTKIEERKDNKSKIIIDSISTNINITNDIEYIISIFALLNSNTYINKIRNRFFATSVWLEKQNGTKEYDYENIISILDEVLLINLLRENNITDILDLKNAIIDAKIVENDETYIKNYIFESNEEKLKKRLFCEELVDKEDIRKIFEDENFKFKSFIKKNEIGNFKINFGILSNLECNSEVEAKKIIGKNSKKLESFIQNIIDEYKSNIRTLFSSEFLEKYKEEIDNLVEDTESENKNKFEKIYYPKEHKNELYIYKKNLFLNIGNPNFDKIYGLISKDIKNVDTKILFDDDIKKNKISEIDAILKNLNDKLNGYSNDYKAKYVNKLKENDDFFAKNIQNENYSSFGEFEKDYNKVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFRLIGNNDILERLMKPKKVSVLELESYNSDYIKNLIIELLTKIENTNDTL

SEQ ID NO: 33 - protein, 1224 aa, Bergeyella zoohelcum
MENKTSLGNNIYYNPFKPQDKSYFAGYFNAAMENTDSVFRELGKRLKGKEYTSENFFDAIFKENISLVEYERYVKLLSDYFPMARLLDKKEVPIKERKENFKKNFKGIIKAVRDLRNFYTHKEHGEVEITDEIFGVLDEMLKSTVLTVKKKKVKTDKTKEILKKSIEKQLDILCQKKLEYLRDTARKIEEKRRNQRERGEKELVAPFKYSDKRDDLIAAIYNDAFDVYIDKKKDSLKESSKAKYNTKSDPQQEEGDLKIPISKNGVVFLLSLFLTKQEIHAFKSKIAGFKATVIDEATVSEATVSHGKNSICFMATHEIFSHLAYKKLKRKVRTAEINYGEAENAEQLSVYAKETLMMQMLDELSKVPDVVYQNLSEDVQKTFIEDWNEYLKENNGDVGTMEEEQVIHPVIRKRYEDKFNYFAIRFLDEFAQFPTLRFQVHLGNYLHDSRPKENLISDRRIKEKITVFGRLSELEHKKALFIKNTETNEDREHYWEIFPNPNYDFPKENISVNDKDFPIAGSILDREKQPVAGKIGIKVKLLNQQYVSEVDKAVKAHQLKQRKASKPSIQNIIEEIVPINESNPKEAIVFGGQPTAYLSMNDIHSILYEFFDKWEKKKEKLEKKGEKELRKEIGKELEKKIVGKIQAQIQQIIDKDTNAKILKPYQDGNSTAIDKEKLIKDLKQEQNILQKLKDEQTVREKEYNDFIAYQDKNREINKVRDRNHKQYLKDNLKRKYPEAPARKEVLYYREKGKVAVWLANDIKRFMPTDFKNEWKGEQHSLLQKSLAYYEQCKEELKNLLPEKVFQHLPFKLGGYFQQKYLYQFYTCYLDKRLEYISGLVQQAENFKSENKVFKKVENECFKFLKKQNYTHKELDARVQSILGYPIFLERGFMDEKPTIIKGKTFKGNEALFADWFRYYKEYQNFQTFYDTENYPLVELEKKQADRKRKTKIYQQKKNDVFTLLMAKHIFKSVFKQDSIDQFSLEDLYQSREERLGNQERARQTGERNTNYIWNKTVDLKLCDGKITVENVKLKNVGDFIKYEYDQRVQAFLKYEENIEWQAFLIKESKEEENYPYVVEREIEQYEKVRREELLKEVHLIEEYILEKVKDKEILKKGDNQNFKYYILNGLLKQLKNEDVESYKVFNLNTEPEDVNINQLKQEATDLEQKAFVLTYIRNKFAHNQLPKKEFWDYCQEKYGKIEKEKTYAEYFAEVFKKEKEALIK

SEQ ID NO: 34 - protein, 1126 aa, Prevotella intermedia
MEDDKKTTDSIRYELKDKHFWAAFLNLARHNVYITVNHINKILEEGEINRDGYETTLKNTWNEIKDINKKDRLSKLIIKHFPFLEAATYRLNPTDTTKQKEEKQAEAQSLESLRKSFFVFIYKLRDLRNHYSHYKHSKSLERPKFEEGLLEKMYNIFNASIRLVKEDYQYNKDINPDEDFKHLDRTEEEFNYYFTKDNEGNITESGLLFFVSLFLEKKDAIWMQQKLRGFKDNRENKKKMTNEVFCRSRMLLPKLRLQSTQTQDWILLDMLNELIRCPKSLYERLREEDREKFRVPIEIADEDYDAEQEPFKNTLVRHQDRFPYFALRYFDYNEIFTNLRFQIDLGTYHFSIYKKQIGDYKESHHLTHKLYGFERIQEFTKQNRPDEWRKFVKTFNSFETSKEPYIPETTPHYHLENQKIGIRFRNDNDKIWPSLKTNSEKNEKSKYKLDKSFQAEAFLSVHELLPMMFYYLLLKTENTDNDNEIETKKKENKNDKQEKHKIEEIIENKITEIYALYDTFANGEIKSIDELEEYCKGKDIEIGHLPKQMIAILKDEHKVMATEAERKQEEMLVDVQKSLESLDNQINEEIENVERKNSSLKSGKIASWLVNDMMRFQPVQKDNEGKPLNNSKANSTEYQLLQRTLAFFGSEHERLAPYFKQTKLIESSNPHPFLKDTEWEKCNNILSFYRSYLEAKKNFLESLKPEDWEKNQYFLKLKEPKTKPKTLVQGWKNGFNLPRGIFTEPIRKWFMKHRENITVAELKRVGLVAKVIPLFFSEEYKDSVQPFYNYHFNVGNINKPDEKNFLNCEERRELLRKKKDEFKKMTDKEKEENPSYLEFKSWNKFERELRLVRNQDIVTWLLCMELFNKKKIKELNVEKIYLKNINTNTTKKEKNTEEKNGEEKNIKEKNNILNRIMPMRLPIKVYGRENFSKNKKKKIRRNTFFTVYIEEKGTKLLKQGNFKALERDRRLGGLFSFVKTPSKAESKSNTISKLRVEYELGEYQKARIEIIKDMLALEKTLIDKYNSLDTDNFNKMLTDWLELKGEPDKASFQNDVDLLIAVRNAFSHNQYPMRNRIAFANINPFSLSSANTSEEKGLGIANQLKDKTHKTIEKIIEIEKPIETKE

SEQ ID NO: 35 - protein, 1127 aa, Prevotella buccae
MQKQDKLFVDRKKNAIFAFPKYITIMENKEKPEPIYYELTDKHFWAAFLNLARHNVYTTINHINRRLEIAELKDDGYMMGIKGSWNEQAKKLDKKVRLRDLIMKHFPFLEAAAYEMTNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYNAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSAHNNLFPSLQTDKTCNGRSKFNLGTQFTAEAFLSVHELLPMMFYYLLLTKDYSRKESADKVEGIIRKEISNIYAIYDAFANNEINSIADLTRRLQNTNILQGHLPKQMISILKGRQKDMGKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNRLKPKKRQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKALVKDRRLNGLFSFAETTDLNLEEHPISKLSVDLELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 36 - protein, 953 aa, Alistipes sp.
MSNEIGAFREHQFAYAPGNEKQEEATFATYFNLALSNVEGMMFGEVESNPDKIEKSLDTLPPAILRQIASFIWLSKEDHPDKAYSTEEVKVIVTDLVRRLCFYRNYFSHCFYLDTQYFYSDELVDTTAIGEKLPYNFHHFITNRLFRYSLPEITLFRWNEGERKYEILRDGLIFFCCLFLKRGQAERFLNELRFFKRTDEEGRIKRTIFTKYCTRESHKHIGIEEQDFLIFQDIIGDLNRVPKVCDGVVDLSKENERYIKNRETSNESDENKARYRLLIREKDKFPYYLMRYIVDFGVLPCITFKQNDYSTKEGRGQFHYQDAAVAQEERCYNFVVRNGNVYYSYMPQAQNVVRISELQGTISVEELRNMVYASINGKDVNKSVEQYLYHLHLLYEKILTISGQTIKEGRVDVEDYRPLLDKLLLRPASNGEELRRELRKLLPKRVCDLLSNRFDCSEGVSAVEKRLKAILLRHEQLLLSQNPALHIDKIKSVIDYLYLFFSDDEKFRQQPTEKAHRGLKDEEFQMYHYLVGDYDSHPLALWKELEASGRLKPEMRKLTSATSLHGLYMLCLKGTVEWCRKQLMSIGKGTAKVEAIADRVGLKLYDKLKEYTPEQLEREVKLVVMHGYAAAATPKPKAQAAIPSKLTELRFYSFLGKREMSFAAFIRQDKKAQKLWLRNFYTVENIKTLQKRQAAADAACKKLYNLVGEVERVHTNDKVLVLVAQRYRERLLNVGSKCAVTLDNPERQQKLADVYEVQNAWLSIRFDDLDFTLTHVNLSNLRKAYNLIPRKHILAFKEYLDNRVKQKLCEECRNVRRKEDLCTCCSPRYSNLTSWLKENHSESSIEREAATMMLLDVERKLLSFLLDERRKAIIEYGKFIPFSALVKECRLADAGLCGIRNDVLHDNVISYADAIGKLSAYFPKEASEAVEYIRRTKEVREQRREELMANSSQ

SEQ ID NO: 37 - protein, 1322 aa, Prevotella sp.
MSKECKKQRQEKKRRLQKANFSISLTGKHVFGAYFNMARTNFVKTINYILPIAGVRGNYSENQINKMLHALFLIQAGRNEELTTEQKQWEKKLRLNPEQQTKFQKLLFKHFPVLGPMMADVADHKAYLNKKKSTVQTEDETFAMLKGVSLADCLDIICLMADTLTECRNFYTHKDPYNKPSQLADQYLHQEMIAKKLDKVVVASRRILKDREGLSVNEVEFLTGIDHLHQEVLKDEFGNAKVKDGKVMKTFVEYDDFYFKISGKRLVNGYTVTTKDDKPVNVNTMLPALSDFGLLYFCVLFLSKPYAKLFIDEVRLFEYSPFDDKENMIMSEMLSIYRIRTPRLHKIDSHDSKATLAMDIFGELRRCPMELYNLLDKNAGQPFFHDEVKHPNSHTPDVSKRLRYDDRFPTLALRYIDETELFKRIRFQLQLGSFRYKFYDKENCIDGRVRVRRIQKEINGYGRMQEVADKRMDKWGDLIQKREERSVKLEHEELYINLDQFLEDTADSTPYVTDRRPAYNIHANRIGLYWEDSQNPKQYKVFDENGMYIPELVVTEDKKAPIKMPAPRCALSVYDLPAMLFYEYLREQQDNEFPSAEQVIIEYEDDYRKFFKAVAEGKLKPFKRPKEFRDFLKKEYPKLRMADIPKKLQLFLCSHGLCYNNKPETVYERLDRLTLQHLEERELHIQNRLEHYQKDRDMIGNKDNQYGKKSFSDVRHGALARYLAQSMMEWQPTKLKDKEKGHDKLTGLNYNVLTAYLATYGHPQVPEEGFTPRTLEQVLINAHLIGGSNPHPFINKVLALGNRNIEELYLHYLEEELKHIRSRIQSLSSNPSDKALSALPFIHHDRMRYHERTSEEMMALAARYTTIQLPDGLFTPYILEILQKHYTENSDLQNALSQDVPVKLNPTCNAAYLITLFYQTVLKDNAQPFYLSDKTYTRNKDGEKAESFSFKRAYELFSVLNNNKKDTFPFEMIPLFLTSDEIQERLSAKLLDGDGNPVPEVGEKGKPATDSQGNTIWKRRIYSEVDDYAEKLTDRDMKISFKGEWEKLPRWKQDKIIKRRDETRRQMRDELLQRMPRYIRDIKDNERTLRRYKTQDMVLFLLAEKMFTNIISEQSSEFNWKQMRLSKVCNEAFLRQTLTFRVPVTVGETTIYVEQENMSLKNYGEFYRFLTDDRLMSLLNNIVETLKPNENGDLVIRHTDLMSELAAYDQYRSTIFMLIQSIENLIITNNAVLDDPDADGFWVREDLPKRNNFASLLELINQLNNVELTDDERKLLVAIRNAFSHNSYNIDFSLIKDVKHLPEVAKGILQHLQSMLGVEITK

SEQ ID NO: 38 - protein, 1095 aa, Riemerella anatipestifer
MEKPLLPNVYTLKHKFFWGAFLNIARHNAFITICHINEQLGLKTPSNDDKIVDVVCETWNNILNNDHDLLKKSQLTELILKHFPFLTAMCYHPPKKEGKKKGHQKEQQKEKESEAQSQAEALNPSKLIEALEILVNQLHSLRNYYSHYKHKKPDAEKDIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYHKTPIRTVYIREEHTKALKMGNFKALVKDRRLNGLFSFIKEENDTQKHPISQLRLRRELEIYQSLRVDAFKETLSLEEKLLNKHTSLSSLENEFRALLEEWKKEYAASSMVTDEHIAFIASVRNAFCHNQYPFYKEALHAPIPLFTVAQPTTEEKDGLGIAEALLKVLREYCEIVKSQI

SEQ ID NO: 39 - protein, 1124 aa, Prevotella aurantiaca
MEDDKKTTGSISYELKDKHFWAAFLNLARHNVYITINHINKLLEIREIDNDEKVLDIKTLWQKGNKDLNQKARLRELMTKHFPFLETAIYTKNKEDKKEVKQEKQAEAQSLESLKDCLFLFLDKLQEARNYYSHYKYSEFSKEPEFEEGLLEKMYNIFGNNIQLVINDYQHNKDINPDEDFKHLDRKGQFKYSFADNEGNITESGLLFFVSLFLEKKDAIWMQQKLNGFKDNLENKKKMTHEVFCRSRILMPKLRLESTQTQDWILLDMLNELIRCPKSLYERLQGDDREKFKVPFDPADEDYNAEQEPFKNTLIRHQDRFPYFVLRYFDYNEIFKNLRFQIDLGTYHFSIYKKLIGGQKEDRHLTHKLYGFERIQEFAKQNRPDEWKAIVKDLDTYETSNKRYISETTPHYHLENQKIGIRFRNGNKEIWPSLKTNDENNEKSKYKLDKQYQAEAFLSVHELLPMMFYYLLLKKEKPNNDEINASIVEGFIKREIRNIFKLYDAFANGEINNIDDLEKYCADKGIPKRHLPKQMVAILYDEHKDMVKEAKRKQKEMVKDTKKLLATLEKQTQKEKEDDGRNVKLLKSGEIARWLVNDMMRFQPVQKDNEGKPLNNSKANSTEYQMLQRSLALYNNEEKPTRYFRQVNLIESNNPHPFLKWTKWEECNNILTFYYSYLTKKIEFLNKLKPEDWKKNQYFLKLKEPKTNRETLVQGWKNGFNLPRGIFTEPIREWFKRHQNNSKEYEKVEALDRVGLVTKVIPLFFKEEYFKDKEENFKEDTQKEINDCVQPFYNFPYNVGNIHKPKEKDFLHREERIELWDKKKDKFKGYKEKIKSKKLTEKDKEEFRSYLEFQSWNKFERELRLVRNQDIVTWLLCKELIDKLKIDELNIEELKKLRLNNIDTDTAKKEKNNILNRVMPMELPVTVYEIDDSHKIVKDKPLHTIYIKEAETKLLKQGNFKALVKDRRLNGLFSFVKTNSEAESKRNPISKLRVEYELGEYQEARIEIIQDMLALEEKLINKYKDLPTNKFSEMLNSWLEGKDEADKARFQNDVDFLIAVRNAFSHNQYPMHNKIEFANIKPFSLYTANNSEEKGLGIANQLKDKTKETTDKIKKIEKPIETKE

SEQ ID NO: 40 - protein, 1151 aa, Prevotella saccharolytica
MEDKPFWAAFFNLARHNVYLTVNHINKLLDLEKLYDEGKHKEIFEREDIFNISDDVMNDANSNGKKRKLDIKKIWDDLDTDLTRKYQLRELILKHFPFIQPAIIGAQTKERTTIDKDKRSTSTSNDSLKQTGEGDINDLLSLSNVKSMFFRLLQILEQLRNYYSHVKHSKSATMPNFDEDLLNWMRYIFIDSVNKVKEDYSSNSVIDPNTSFSHLIYKDEQGKIKPCRYPFTSKDGSINAFGLLFFVSLFLEKQDSIWMQKKIPGFKKASENYMKMTNEVFCRNHILLPKIRLETVYDKDWMLLDMLNEVVRCPLSLYKRLTPAAQNKFKVPEKSSDNANRQEDDNPFSRILVRHQNRFPYFVLRFFDLNEVFTTLRFQINLGCYHFAICKKQIGDKKEVHHLIRTLYGFSRLQNFTQNTRPEEWNTLVKTTEPSSGNDGKTVQGVPLPYISYTIPHYQIENEKIGIKIFDGDTAVDTDIWPSVSTEKQLNKPDKYTLTPGFKADVFLSVHELLPMMFYYQLLLCEGMLKTDAGNAVEKVLIDTRNAIFNLYDAFVQEKINTITDLENYLQDKPILIGHLPKQMIDLLKGHQRDMLKAVEQKKAMLIKDTERRLKLLDKQLKQETDVAAKNTGTLLKNGQIADWLVNDMMRFQPVKRDKEGNPINCSKANSTEYQMLQRAFAFYATDSCRLSRYFTQLHLIHSDNSHLFLSRFEYDKQPNLIAFYAAYLKAKLEFLNELQPQNWASDNYFLLLRAPKNDRQKLAEGWKNGFNLPRGLFTEKIKTWFNEHKTIVDISDCDIFKNRVGQVARLIPVFFDKKFKDHSQPFYRYDFNVGNVSKPTEANYLSKGKREELFKSYQNKFKNNIPAEKTKEYREYKNFSLWKKFERELRLIKNQDILIWLMCKNLFDEKIKPKKDILEPRIAVSYIKLDSLQTNTSTAGSLNALAKVVPMTLAIHIDSPKPKGKAGNNEKENKEFTVYIKEEGTKLLKWGNFKTLLADRRIKGLFSYIEHDDIDLKQHPLTKRRVDLELDLYQTCRIDIFQQTLGLEAQLLDKYSDLNTDNFYQMLIGWRKKEGIPRNIKEDTDFLKDVRNAFSHNQYPDSKKIAFRRIRKFNPKELILEEEEGLGIATQMYKEVEKVVNRIKRIELFD

SEQ ID NO: 41 - protein, 1120 aa, Prevotella intermedia
MEDDKKTTDSIRYELKDKHFWAAFLNLARHNVYITVNHINKILEEDEINRDGYENTLENSWNEIKDINKKDRLSKLIIKHFPFLEATTYRQNPTDTTKQKEEKQAEAQSLESLKKSFFVFIYKLRDLRNHYSHYKHSKSLERPKFEEDLQNKMYNIFDVSIQFVKEDYKHNTDINPKKDFKHLDRKRKGKFHYSFADNEGNITESGLLFFVSLFLEKKDAIWVQKKLEGFKCSNKSYQKMTNEVFCRSRMLLPKLRLESTQTQDWILLDMLNELIRCPKSLYERLQGVNRKKFYVSFDPADEDYDAEQEPFKNTLVRHQDRFPYFALRYFDYNEVFANLRFQIDLGTYHFSIYKKLIGGQKEDRHLTHKLYGFERIQEFDKQNRPDEWKAIVKDSDTFKKKEEKEEEKPYISETTPHYHLENKKIGIAFKNHNIWPSTQTELTNNKRKKYNLGTSIKAEAFLSVHELLPMMFYYLLLKTENTKNDNKVGGKKETKKQGKHKIEAIIESKIKDIYALYDAFANGEINSEDELKEYLKGKDIKIVHLPKQMIAILKNEHKDMAEKAEAKQEKMKLATENRLKTLDKQLKGKIQNGKRYNSAPKSGEIASWLVNDMMRFQPVQKDENGESLNNSKANSTEYQLLQRTLAFFGSEHERLAPYFKQTKLIESSNPHPFLNDTEWEKCSNILSFYRSYLKARKNFLESLKPEDWEKNQYFLMLKEPKTNRETLVQGWKNGFNLPRGFFTEPIRKWFMEHWKSIKVDDLKRVGLVAKVTPLFFSEKYKDSVQPFYNYPFNVGDVNKPKEEDFLHREERIELWDKKKDKFKGYKAKKKFKEMTDKEKEEHRSYLEFQSWNKFERELRLVRNQDIVTWLLCTELIDKLKIDELNIKELKKLRLKDINTDTAKKEKNNILNRVMPMELPVTVYKVNKGGYIIKNKPLHTIYIKEAETKLLKQGNFKALVKDRRLNGLFSFVKTPSEAESESNPISKLRVEYELGKYQNARLDIIEDMLALEKKLIDKYNSLDTDNFHNMLTGWLELKGEAKKARFQNDVKLLTAVRNAFSHNQYPMYDENLFGNIERFSLSSSNIIESKGLDIAAKLKEEVSKAAKKIQNEEDNKKEKET

SEQ ID NO: 42 - protein, 1199 aa, Capnocytophaga canimorsus
MKNIQRLGKGNEFSPFKKEDKFYFGGFLNLANNNIEDFFKEIITRFGIVITDENKKPKETFGEKILNEIFKKDISIVDYEKWVNIFADYFPFTKYLSLYLEEMQFKNRVICFRDVMKELLKTVEALRNFYTHYDHEPIKIEDRVFYFLDKVLLDVSLTVKNKYLKTDKTKEFLNQHIGEELKELCKQRKDYLVGKGKRIDKESEIINGIYNNAFKDFICKREKQDDKENHNSVEKILCNKEPQNKKQKSSATVWELCSKSSSKYTEKSFPNRENDKHCLEVPISQKGIVFLLSFFLNKGEIYALTSNIKGFKAKITKEEPVTYDKNSIRYMATHRMFSFLAYKGLKRKIRTSEINYNEDGQASSTYEKETLMLQMLDELNKVPDVVYQNLSEDVQKTFIEDWNEYLKENNGDVGTMEEEQVIHPVIRKRYEDKFNYFAIRFLDEFAQFPTLRFQVHLGNYLCDKRTKQICDTTTEREVKKKITVFGRLSELENKKAIFLNEREEIKGWEVFPNPSYDFPKENISVNYKDFPIVGSILDREKQPVSNKIGIRVKIADELQREIDKAIKEKKLRNPKNRKANQDEKQKERLVNEIVSTNSNEQGEPVVFIGQPTAYLSMNDIHSVLYEFLINKISGEALETKIVEKIETQIKQIIGKDATTKILKPYTNANSNSINREKLLRDLEQEQQILKTLLEEQQQREKDKKDKKSKRKHELYPSEKGKVAVWLANDIKRFMPKAFKEQWRGYHHSLLQKYLAYYEQSKEELKNLLPKEVFKHFPFKLKGYFQQQYLNQFYTDYLKRRLSYVNELLLNIQNFKNDKDALKATEKECFKFFRKQNYIINPINIQIQSILVYPIFLKRGFLDEKPTMIDREKFKENKDTELADWFMHYKNYKEDNYQKFYAYPLEKVEEKEKFKRNKQINKQKKNDVYTLMMVEYIIQKIFGDKFVEENPLVLKGIFQSKAERQQNNTHAATTQERNLNGILNQPKDIKIQGKITVKGVKLKDIGNFRKYEIDQRVNTFLDYEPRKEWMAYLPNDWKEKEKQGQLPPNNVIDRQISKYETVRSKILLKDVQELEKIISDEIKEEHRHDLKQGKYYNFKYYILNGLLRQLKNENVENYKVFKLNTNPEKVNITQLKQEATDLEQKAFVLTYIRNKFAHNQLPKKEFWDYCQEKYGKIEKEKTYAEYFAEVFKREKEALIK

SEQ ID NO: 43 - protein, 1175 aa, Porphyromonas gulae
MTEQSERPYNGTYYTLEDKHFWAAFLNLARHNAYITLTHIDRQLAYSKADITNDQDVLSFKALWKNFDNDLERKSRLRSLILKHFSFLEGAAYGKKLFESKSSGNKSSKNKELTKKEKEELQANALSLDNLKSILFDFLQKLKDFRNYYSHYRHSGSSELPLFDGNMLQRLYNVFDVSVQRVKIDHEHNDEVDPHYHFNHLVRKGKKDRYGHNDNPSFKHHFVDGEGMVTEAGLLFFVSLFLEKRDAIWMQKKIRGFKGGTETYQQMTNEVFCRSRISLPKLKLESLRMDDWMLLDMLNELVRCPKPLYDRLREDDRACFRVPVDILPDEDDTDGGGEDPFKNTLVRHQDRFPYFALRYFDLKKVFTSLRFHIDLGTYHFAIYKKMIGEQPEDRHLTRNLYGFGRIQDFAEEHRPEEWKRLVRDLDYFETGDKPYISQTSPHYHIEKGKIGLRFMPEGQHLWPSPEVGTTRTGRSKYAQDKRLTAEAFLSVHELMPMMFYYFLLREKYSEEVSAERVQGRIKRVIEDVYAVYDAFARDEINTRDELDACLADKGIRRGHLPRQMIAILSQEHKDMEEKIRKKLQEMMADTDHRLDMLDRQTDRKIRIGRKNAGLPKSGVIADWLVRDMMRFQPVAKDASGKPLNNSKANSTEYRMLQRALALFGGEKERLTPYFRQMNLTGGNNPHPFLHETRWESHTNILSFYRSYLRARKAFLERIGRSDRVENRPFLLLKEPKTDRQTLVAGWKGEFHLPRGIFTEAVRDCLIEMGHDEVASYKEVGFMAKAVPLYFERACEDRVQPFYDSPFNVGNSLKPKKGRFLSKEERAEEWERGKERFRDLEAWSYSAARRIEDAFAGIEYASPGNKKKIEQLLRDLSLWEAFESKLKVRADRINLAKLKKEILEAQEHPYHDFKSWQKFERELRLVKNQDIITWMMCRDLMEENKVEGLDTGTLYLKDIRPNVQEQGSLNVLNRVKPMRLPVVVYRADSRGHVHKEEAPLATVYIEERDTKLLKQGNFKSFVKDRRLNGLFSFVDTGGLAMEQYPISKLRVEYELAKYQTARVCVFELTLRLEESLLTRYPHLPDESFREMLESWSDPLLAKWPELHGKVRLLIAVRNAFSHNQYPMYDEAVFSSIRKYDPSSPDAIEERMGLNIAHRLSEEVKQAKETVERIIQA

SEQ ID NO: 44 - protein, 1090 aa, Prevotella sp.
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMK

SEQ ID NO: 45 - protein, 1150 aa, Flavobacterium branchiophilum
MENLNKILDKENEICISKIFNTKGIAAPITEKALDNIKSKQKNDLNKEARLHYFSIGHSFKQIDTKKVFDYVLIEELKDEKPLKFITLQKDFFTKEFSIKLQKLINSIRNINNHYVHNFNDINLNKIDSNVFHFLKESFELAIIEKYYKVNKKYPLDNEIVLFLKELFIKDENTALLNYFTNLSKDEAIEYILTFTITENKIWNINNEHNILNIEKGKYLTFEAMLFLITIFLYKNEANHLLPKLYDFKNNKSKQELFTFFSKKFTSQDIDAEEGHLIKFRDMIQYLNHYPTAWNNDLKLESENKNKIMTTKLIDSIIEFELNSNYPSFATDIQFKKEAKAFLFASNKKRNQTSFSNKSYNEEIRHNPHIKQYRDEIASALTPISFNVKEDKFKIFVKKHVLEEYFPNSIGYEKFLEYNDFTEKEKEDFGLKLYSNPKTNKLIERIDNHKLVKSHGRNQDRFMDFSMRFLAENNYFGKDAFFKCYKFYDTQEQDEFLQSNENNDDVKFHKGKVTTYIKYEEHLKNYSYWDCPFVEENNSMSVKISIGSEEKILKIQRNLMIYFLENALYNENVENQGYKLVNNYYRELKKDVEESIASLDLIKSNPDFKSKYKKILPKRLLHNYAPAKQDKAPENAFETLLKKADFREEQYKKLLKKAEHEKNKEDFVKRNKGKQFKLHFIRKACQMMYFKEKYNTLKEGNAAFEKKDPVIEKRKNKEHEFGHHKNLNITREEFNDYCKWMFAFNGNDSYKKYLRDLFSEKHFFDNQEYKNLFESSVNLEAFYAKTKELFKKWIETNKPTNNENRYTLENYKNLILQKQVFINVYHFSKYLIDKNLLNSENNVIQYKSLENVEYLISDFYFQSKLSIDQYKTCGKLFNKLKSNKLEDCLLYEIAYNYIDKKNVHKIDIQKILTSKIILTINDANTPYKISVPFNKLERYTEMIAIKNQNNLKARFLIDLPLYLSKNKIKKGKDSAGYEIIIKNDLEIEDINTINNKIINDSVKFTEVLMELEKYFILKDKCILSKNYIDNSEIPSLKQFSKVWIKENENEIINYRNIACHFHLPLLETFDNLLLNVEQKFIKEELQNVSTINDLSKPQEYLILLFIKFKHNNFYLNLFNKNESKTIKNDKEVKKNRVLQKFINQVILKKK

SEQ ID NO: 46 - protein, 1119 aa, Porphyromonas gingivalis
MTEQNEKPYNGTYYTLEDKHFWAAFLNLARHNAYITLAHIDRQLAYSKADITNDEDILFFKGQWKNLDNDLERKARLRSLILKHFSFLEGAAYGKKLFESQSSGNKSSKKKELSKKEKEELQANALSLDNLKSILFDFLQKLKDFRNYYSHYRHPESSELPLFDGNMLQRLYNVFDVSVQRVKRDHEHNDKVDPHRHFNHLVRKGKKDKYGNNDNPFFKHHFVDREGTVTEAGLLFFVSLFLEKRDAIWMQKKIRGFKGGTEAYQQMTNEVFCRSRISLPKLKLESLRTDDWMLLDMLNELVRCPKSLYDRLREEDRARFRVPVDILSDEDDTDGTEEDPFKNTLVRHQDRFPYFALRYFDLKKVFTSLRFHIDLGTYHFAIYKKNIGEQPEDRHLTRNLYGFGRIQDFAEEHRPEEWKRLVRDLDYFETGDKPYITQTTPHYHIEKGKIGLRFVPEGQHLWPSPEVGATRTGRSKYAQDKRLTAEAFLSVHELMPMMFYYFLLREKYSEEVSAEKVQGRIKRVIEDVYAVYDAFARDEINTRDELDACLADKGIRRGHLPRQMIAILSQEHKDMEEKVRKKLQEMIADTDHRLDMLDRQTDRKIRIGRKNAGLPKSGVVADWLVRDMMRFQPVAKDTSGKPLNNSKANSTEYRMLQRALALFGGEKERLTPYFRQMNLTGGNNPHPFLHETRWESHTNILSFYRSYLEARKAFLQSIGRSDRVENHRFLLLKEPKTDRQTLVAGWKGEFHLPRGIFTEAVRDCLIEMGYDEVGSYKEVGFMAKAVPLYFERASKDRVQPFYDYPFNVGNSLKPKKGRFLSKEKRAEEWESGKERFRLAKLKKEILEAKEHPYHDFKSWQKFERELRLVKNQDIITWMMCRDLMEENKVEGLDTGTLYLKDIRTDVQEQGSLNVLNRVKPMRLPVVVYRADSRGHVHKEQAPLATVYIEERDTKLLKQGNFKSFVKDRRLNGLFSFVDTGALAMEQYPISKLRVEYELAKYQTARVCAFEQTLELEESLLTRYPHLPDKNFRKMLESWSDPLLDKWPDLHGNVRLLIAVRNAFSHNQYPMYDETLFSSIRKYDPSSPDAIEERMGLNIAHRLSEEVKQAKEMVERIIQA

SEQ ID NO: 47 - protein, 1132 aa, Prevotella intermedia
MEDDKKTKESTNMLDNKHFWAAFLNLARHNVYITVNHINKVLELKNKKDQDIIIDNDQDILAIKTHWEKVNGDLNKTERLRELMTKHFPFLETAIYTKNKEDKEEVKQEKQAKAQSFDSLKHCLFLFLEKLQEARNYYSHYKYSESTKEPMLEKELLKKMYNIFDDNIQLVIKDYQHNKDINPDEDFKHLDRTEEEFNYYFTTNKKGNITASGLLFFVSLFLEKKDAIWMQQKLRGFKDNRESKKKMTHEVFCRSRMLLPKLRLESTQTQDWILLDMLNELIRCPKSLYERLQGEYRKKFNVPFDSADEDYDAEQEPFKNTLVRHQDRFPYFALRYFDYNEIFTNLRFQIDLGTYHFSIYKKLIGGQKEDRHLTHKLYGFERIQEFAKQNRTDEWKAIVKDFDTYETSEEPYISETAPHYHLENQKIGIRFRNDNDEIWPSLKTNGENNEKRKYKLDKQYQAEAFLSVHELLPMMFYYLLLKKEEPNNDKKNASIVEGFIKREIRDIYKLYDAFANGEINNIDDLEKYCEDKGIPKRHLPKQMVAILYDEHKDMAEEAKRKQKEMVKDTKKLLATLEKQTQGEIEDGGRNIRLLKSGEIARWLVNDMMRFQPVQKDNEGNPLNNSKANSTEYQMLQRSLALYNKEEKPTRYFRQVNLINSSNPHPFLKWTKWEECNNILSFYRSYLTKKIEFLNKLKPEDWEKNQYFLKLKEPKTNRETLVQGWKNGFNLPRGIFTEPIREWFKRHQNDSEEYEKVETLDRVGLVTKVIPLFFKKEDSKDKEEYLKKDAQKEINNCVQPFYGFPYNVGNIHKPDEKDFLPSEERKKLWGDKKYKFKGYKAKVKSKKLTDKEKEEYRSYLEFQSWNKFERELRLVRNQDIVTWLLCTELIDKLKVEGLNVEELKKLRLKDIDTDTAKQEKNNILNRVMPMQLPVTVYEIDDSHNIVKDRPLHTVYIEETKTKLLKQGNFKALVKDRRLNGLFSFVDTSSETELKSNPISKSLVEYELGEYQNARIETIKDMLLLEETLIEKYKTLPTDNFSDMLNGWLEGKDEADKARFQNDVKLLVAVRNAFSHNQYPMRNRIAFANINPFSLSSADTSEEKKLDIANQLKDKTHKIIKRIIEIEKPIETKE

SEQ ID NO: 48 - protein, 804 aa, unidentified
MEFENIKKTSNKEVYSIEQYEGEKKWCFAIVLNRAQTNLEENPKLFEQTLTRFEKIMKQDWFNEETKKLIYEKEEENKVKEEIQIAASERLKNLRNYFSHYLHAPDCLIFNRNDTIRIIMEKAYEKSRFEAKKKQQEDISIEFPELFEEEDKITSAGVVFFVSFFIERRFLNRLMGYVQGFRKTEGEYNITRQVFSKYCLKDSYSVQAQDHDAVMFRDILGYLSRVPTEIYQHIKLTRKRSQDQLSERKTDKFILFALKYLEDYGLKDLADYTACFARSKIKRENEDTKETDGNKHKFHREKPVVEIHFDKEKQDQFYIKRNNVILKAQKKGGQSNVFRMGVYELKYLVLLSLLGKAEEAIQRIDRYISSLKKQLPYLDKISNEEIQKSINFLPRFVRSRLGLLQVDDEKRLKTRLEYVKAKWTDKKEGSRKLELHRKGRDILRYINERCDRPLSRKEYNNILKFIVNKDFAGFYNELEELKRTRRLDKNIIQKLSGHTTLNALHERVCDLVLQELGSLQSENLKEYIGLIPKEEKEVTFREKVDRILEQPVVYKGFLRYEFFKEDKKSFARLVEEAIKTKWSDFDIPLGEEYYNIPSLDRFDRTNKKLYETLAMDRLCLMMARQYYLRLNEKLAEKAQHIYWKKEDGREVIIFKFQNPKEQKKSFSIRFSILDYTKMYVMDDPEFLSRLWEYFIPKEAKEIDYHKHYARAFDKYTNLQKEGIDAILKLEGRIIERRKIKPAKNYIEFQEIMNRSGYNNDQQVALKRVRNALLHYNLNFEREHLKRFYGVVKREGIEKKWSLIV

SEQ ID NO: 49 - protein, 801 aa, unidentified
MQVENIKKGSSQGMYSIEQYEGAKKWCFAIVLNRAQTNLQGNPKLFEETLTRFERIRKEDWFDQETKKLIYAKQEQNEVEEEIQKAADEKLRDLRNYFSHYFHTPDCLIFTQNDPVRIIMEKAYEKARFEQAKKEQEDISIEFGELFEENGRITSAGVVFFASFFAERRFLNRLMGYVQGFTRTEGEYKITRDVFSTYCLRDSYSVKTPDHDAVMFRDILGYLSRVPSESYQRIKESQMRSETQLSERKTDKFILFALNYLEDYGLEDLADYTACFARTRIKREQDENTDGKEQKPHRKKPRVEIHFERAEGDPFYIKHNNVILRTQKKGAQTYIFRMGVYELKYLVLLSLLGKGAEAVKRIDRYVHSLRNQLPHIEKKSTEEIEGYVRFLPRFVRSHLGLLGVDDEKKIKARVDYVKAKWLEKKEKSRELQLHRKGRDILRYINERCERPLNIDEYNRILELLVTKHLDGFYRELEELKTRRIDKNIVCNLSRHKSVNALHEKVCDLVVQELESLGREELKEYVGLIPKEEKEVSFEEKTDRVVKQPVIYKGFLRNEFFRESRKSFARLVEEAVREKGEVYDVPLGGEYYEIVSLDTFDKDNKRLYETLAMDRLLLMIARQYHLSLNKELAKRAQQIEWKKEDGEEVIIFTLKNPAQPEQSCSVRFSLRDYTKLYVMDDAEFLARLCDYFLPKDEEQIDYHRLYTQGMNRYTNLQREGIEAILELEKKTIGPEQPRPPKNYIPFSEIMDKSAYNEDDQKALRRVRNALLHHNLNFARADFKRFCGIMKREGIEKRWSLAV

SEQ ID NO: 50 - protein, 775 aa, Planctomyces sp.
MAQVSKQTSKKRELSIDEYQGARKWCFTIAFNKALVNRDKNDGLFVESLLRHEKYSKHDWYDEDTRALIKCSTQAANAKAEALRNYFSHYRHSPGCLTFTAEDELRTIMERAYERAIFECRRRETEVIIEFPSLFEGDRITTAGVVFFVSFFVERRVLDRLYGAVSGLKKNEGQYKLTRKALSMYCLKDSRFTKAWDKRVLLFRDILAQLGRIPAEAYEYYHGEQGDKKRANDNEGTNPKRHKDKFIEFALHYLEAQHSEICFGRRHIVREEAGAGDEHKKHRTKGKVVVDFSKKDEDQSYYISKNNVIVRIDKNAGPRSYRMGLNELKYLVLLSLQGKGDDAIAKLYRYRQHVENILDVVKVTDKDNHVFLPRFVLEQHGIGRKAFKQRIDGRVKHVRGVWEKKKAATNEMTLHEKARDILQYVNENCTRSFNPGEYNRLLVCLVGKDVENFQAGLKRLQLAERIDGRVYSIFAQTSTINEMHQVVCDQILNRLCRIGDQKLYDYVGLGKKDEIDYKQKVAWFKEHISIRRGFLRKKFWYDSKKGFAKLVEEHLESGGGQRDVGLDKKYYHIDAIGRFEGANPALYETLARDRLCLMMAQYFLGSVRKELGNKIVWSNDSIELPVEGSVGNEKSIVFSVSDYGKLYVLDDAEFLGRICEYFMPHEKGKIRYHTVYEKGFRAYNDLQKKCVEAVLAFEEKVVKAKKMSEKEGAHYIDFREILAQTMCKEAEKTAVNKVRRAFFHHHLKFVIDEFGLFSDVMKKYGIEKEWKFPVK

SEQ ID NO: 51 - protein, 797 aa, unidentified
MSGIELKKEEAAFYFNQAELNLKAIEVSIFDEGRRKTLLNNPKILAKVENFIFNSEDVTKNAKGEIDCLLSKLMELRNFYSHYVHKPDVKELSKGEKPILEKYYQFAIDATASADVKLEIIENDTWLTDAGVLLLLCMFLKKSQANKLIGGISGFKRNDPTGQPRRNLFTYYSVREGYKVVPEMQKHFLLFALVNHLSNQDDYIEKAQQPYDIGEGLFFHRIASTFLDISGILRNMKFYTYQSKRLKEQRGELKREKDSFEWIEPFQGNSYFSVDGQKGVIGEDELKELCYALLIGKQDANKVEGRITQFLKKFKNADDAQKVSDDEMLDRGNFPASYFAERRVGSIKDKILSSLEQAIKSYKTSGADVKAYNKMKEVMEFINNSLPVDEKLKRKDYKRYLGMVRLWGSERDNIKREFEAKGWSKYFTSGFWMAKNLERVYGLAREKNAELFNKLKTAVEKMDEREFVKYQQINDAKDLASLRQLANDFGVNWEEKDWEKYSGQIKKQITDSQKLTIMKQRITAGLKRKHGIENLNLRITIDSSKSRKAVLNRIAIPRGFVKKHILDWQGSEKVPKKIREAKCKILLSKEYEELSRQFYKVKDYDKMTQINSLYEKNKLIALMAVYLMEQLRIQLKEHTELRNLDKTTVDFRISDKVTEKIPFSQYPSLVYAMSREYADNVDNYKFSEEDKKKLDKIKKNLFLGKIDIIEKQRMEFIKEVLGFEEYLFDDKIIDRSKFADTATHISFGEIVGELIGKGWDKDKLTKLEYARNKALHGEIPEATSFNEAKQLINELKK

SEQ ID NO: 52 - protein, 905 aa, unidentified
MNPVDIKEASKKAVYYIDQYKGDKKWCFAIVLNRAYDNLEDNPNLLNESLLRFENISLMSWFDFQEDEKQMRDSVFQGYENINKAEQKKEIKLKPDIQKAVVKRLEDLRNYFSHRLHTDDCLTFLKEDPIRFILEKAYEKAKLVHLGRETQESKDFSVPLFEDDKITTAGVILFASFFVEMRILIRVLGLKDVRGFTLTEGKFNLTRKAICHYALPDSYSIKTPRDTKLFRDILGYLSRMPSESYDHYFPEAKKKAEEGEKDKEGEGKDKENKEPQWSKRNTDKFMVFAMRYLEDFQPDVFRICFARQDIQQPQREKDQERKPHKQKGKRKLQFPENLQEARDNPAVRYFIRQNNIDIQIQKNNQKIHCRMGLNELKYLILLCLKGQGGEAIEAIYNEAGQVGNKLPHLAKMRKEGFQDYQKWMPGFVLNHHGLMPEGPNRKEPVAARVGYIRQKWERKRDGSKEARLDSKARDILQYINECGQEYALQQDRERKGTLNIDKYRKIHDFLVNKNIEAFRQELDGLKVDEKVREDLKKRQTVNDLHQRVCNLMIRKLEDLQQSGDLEQLKCYIGLAPAWEYRDEDQTGKEQKEQRFENKVKNLKPMLYRGFLRERFFAEYRSKENKRNFADLVEDVRQKKGEGDVPVDLIYYQIEGDTQEIQVANKKLTETLARDRLCLLIGREYLEQLNRTLSQNAIEERHGPKRKYFIKTEWSKEPVQMGDGKERMRDVIICRIRENPEDENPLCSIRFAVKHWTKLYVMDDPFFLSDVHSYFLSKSNEIDYHTLNQKGICQYTNLQADCMVNILKLEKKVFERVTKRDIDKEKDIKKLINEINNKLQHLPAKKEDNRVPFLLVCEAALEKGILKEKSEIDVVSMVRNSAFHYQLYFSKSEKETFDRIMRREGI

SEQ ID NO: 53 - protein, 789 aa, unidentified
MENIKLEKQKAAFYFNQAELNLKAIEGNIFDKGRRKTLFDNPKILSKVENFIFNFKDVTKNAKGEIDCLLSKLMELRNFYSHYVHKPDVKELSKGEKPLLERYYQIAIEATGSENVKLEIIENDKWLTDAGVLLFLCMFLKKSQANKLISGISGFKRNDTFGQPRRNLFNYFSVRERYKVVPDMQKHFLLFVLVNHLSEQDDYIEKAQQPYNIGEGLFFHRIASTFLNVSGILRNMEFYTYQSKRLKEQRGELKREKDIFTWEEPFQGNSYFEINGHKGVIGEDELKELCYALLSYNKSKYAVEQIEKFLKGFGEVKSEQEIRDSDILNESYFPTNYFAESNIGSIKEKILNRLGKTDDSYKKTGTKIKPYDMMKEVMEFINNSLPADEKLKRKDYRRYLKMVRIWDSEKDNIKREFESKEWSKYFSSNFWMAKNLERVYGLAREKNAELFNKLKAVVEKMDEREFEKYRQINSAEDLASLRRLANDYGVKWEEKDWQEYSGQIKKQISDRQKLTIMKQRITAELKKKHGIENLNLRITIDSNKSRKAVLNRIAVPRGFVKEHILGWQGSEKVSKKTREAKCKILLSKEYEELSKQFFQTRNYDKMTQVNSLYEKNKLIAFMAVYLMGQLNIRFDKPTRLNELEKAEVDFKISDKVTAKIPFSQYPSLVYAMSSKYADSVGSYKFENDEKNKPFLGKIDIIEKQRMEFIKEVLGFEEYLFEKKIIDKSKFADTATHISFREICDELIQKGWDENKLTNLKDARNAALHGEIPAETSFREAKPLINGLKK

SEQ ID NO: 54 - protein, 871 aa, unidentified
MEKYLIKNFEGINKSKFTVALNIANDNCKNKGIQELLKEAQRSKGGITDTQITEVQEHIKERLNSVRNYFSHCYHEKKPLYFEANDPVKIFLEETFAKAVENLQGRFLSDKYKLTVPPLFEPNQNNTITAAGVIFLASFFCHRSYVYRMLGGIPGFKRSDKKKWGDGQKIDYGFTRKLMSFYSLRDSYSVNVQENKELTAFRDILGYLARVPGQAIDWLIEKGKLTKEEGKQFYLGEQSEEREEKAKKEEIKYALRKTDKFMLFAVRFIEDWAEQERIKVEFARYEKMTIVNENKKQDEKEERKVKFVSDEPTAAGWTYYIRNNHAIIKIIPDDKKKKAVSARISENELKYLVLTIIDGNGKNAIAYIGDYIFRTARQIENKSYNAESEKYAPAFVRGGQKKSVDKRIKYIRDEIQQVINDIEAEQEKQKNEQDAPAENRTWLIYKGKKISIILRYVNDNIAEYKKRLSVTEYNELRGYLQQLDFINFHRKLAEYQHHGRLPNGFAESINKFQDLSKLCIEVCERQKKKLQEMAAKGGIELEQYIGLAPKEENQEQNKYATKANNFIKVWLSIPENFLRQKFYDKFCKQQECKNKGSDKPDNTSVPQRKYFIAIIREKNIRPIHADKYYLLGQNPKDYERPDGKIIRQLCDVYCKDGLCMAMAKWYYENRLGKFKDLIEWQTGDDKQQHGYAGHTLEYQATEKIKIRFKLADFTRLDIIEPPERVKNICRQWETELLKKTRDGTISWYDFKLNGLEPYRQWQGYAVADIFWFEESLKINETQWQGRTHMPFNFEKDKPLWCNILDEAVKQNKIEKQDTQALRRVRHDCFHEEFLANYEQLKIFKNLISDKAKDAKPKDKKSRKNEQKYGKR

SEQ ID NO: 55 - protein, 805 aa, unidentified
MKVENIKEKSKKAMYLINHYEGPKKWCFAIVLNRACDNYEDNPHLFSKSLLEFEKTSRKDWFDEETRELVEQADTEIQPNPNLKPNTTANRKLKDIRNYFSHHYHKNECLYFKNDDPIRCIMEAAYEKSKIYIKGKQIEQSDIPLPELFESSGWITPAGILLLASFFVERGILHRLMGNIGGFKDNRGEYGLTHDIFTTYCLKGSYSIRAQDHDAVMFRDILGYLSRVPTESFQRIKQPQIRKEGQLSERKTDKFITFALNYLEDYGLKDLEGCKACFARSKIVREQENVESINDKEYKPHENKKKVEIHFDQSKEDRFYINRNNVILKIQKKDGHSNIVRMGVYELKYLVLMSLVGKAKEAVEKIDNYIQDLRDQLPYIEGKNKEEIKEYVRFFPRFIRSHLGLLQINDEEKIKARLDYVKTKWLDKKEKSKELELHKKGRDILRYINERCDRELNRNVYNRILELLVSKDLTGFYRELEELKRTRRIDKNIVQNLSGQKTINALHEKVCDLVLKEIESLDTENLRKYLGLIPKEEKEVTFKEKVDRILKQPVIYKGFLRYQFFKDDKKSFVLLVEDALKEKGGGCDVPLGKEYYKIVSLDKYDKENKTLCETLAMDRLCLMMARQYYLSLNAKLAQEAQQIEWKKEDSIELIIFTLKNPDQSKQSFSIRFSVRDFTKLYVTDDPEFLARLCSYFFPVEKEIEYHKLYSEGINKYTNLQKEGIEAILELEKKLIERNRIQSAKNYLSFNEIMNKSGYNKDEQDDLKKVRNSLLHYKLIFEKEHLKKFYEVMRGEGIEKKWSLIV

SEQ ID NO: 56 - protein, 838 aa, unidentified
MQFENIKDTGQKPIYSIDQYEGAKKWCFAIVLNRACDNYEDNPQLFSESLLRFEEVNRRDWFDKDIRDLIKKADTEDQIEPKRKPNTPVNRRLHDIRNYFSHSRHQDDCLYFKNDDPMRCIMEAAYEKAKIHIKGRQTEQSDIPLPELFDANNKITSAGVLFLASFFVERGILHRLMGNIGGFKDNRGKYGLTHDIFTTYCLKDSYSIHASDPKVVLFRDIAGYLSLVACEYYPTYLSKIPKENAGGKSSDEEKYAERKTDKFILFALKYLEEFVLPSLKDDYLVDIGRIDIIREESKETEEKDEQYKPHPNQGKVKVVFDSINKELPYYINHNTVILRIQKNGVMAYSCKIGVNDLKYLLLLCLQGKTDKALDAIYNYLHSMQDPPEVVKIGATDKLFQGLPEFILKQSGIKVQDKNKEKAARIKYIRDKWEKKKSESADIELHRKGRDILRYVNWHCETPLGTEKYDQLLVLLVNKNFAGFGDELNQLKRTEIISKDIFEKLSGFKTINTLHQKVCNLVLEELSFFEKSNPEKLEEYIGLIRKPAPENNPPPEYKEKVRRFVEQPMIYKGFLRDQFFVNKDQDGKKLKEQKTFAKLVEETLGQNADVPLGKDFYYVPNIEKDEKKNRFHKDNAVLYETLALDRLCAMMARKCLTQINKNLAEKSEEIDWRNEDGKDFIYLKLVKSDRPQETFKIRFKVNDFAKLYVMDDPDFLGGLMKHFFPQEHSIEYHKLYRNGIERYTDRQKDGIEAILRLEDSVIRQKGMKPKPAKNYISFSEIMAQTDYPEHDQKVLNKVRRALLHYHLKFEPADYNRFVDIMKKDKFWDGERKNEESRGK

SEQ ID NO: 57 - protein, 900 aa, unidentified
MQTATQEQKQKQSIYSILNYQGQRKWCFAIVLNRALDNINPKRETETGKYKNKELFYKSLLRFEGIKKQPWFDETKAEKENVTAKEIIDSKDKAAELLLNLRNYFSHNYHTEKCLYFGTESQHKQIRLIMEAAYERAKAELTGRRTGQEISAEAEKDKDGNIKKYKLSDVPWPPLFDEKDIITTAGVVFFASFFTEAGQIFRLMNWINGLKRNDDKFNITRRALSFYSLPDSYAEAIAEYEVEEDGASRTIRYKAKIFKDILNYLRRIPSETYKLYHSGEENKISGKKEEKGEDENTPVERKTDKFAEFAMRYLEDFEGVRFARYRINTKTRENEVFFDEDELKKLIDKKGVPEQEKDKKFEDYRYYYVKNNAILKTEKGSIRIGINELKYFVLLSLDKMGQQAKEKINSFLSKFTGDNLGNREFIKANIEELPPFILKKFDPLAEDKEKRIEKRVDYLIRKWKRKKEDYEKMRINDKVGGILRYVNENLKPGKKLNAEGYKYLQKLLTMERFSEFEKEINKFEDERESRLKRGALSEIERLKSIDKMFLKVCSIVLKKLESLEGDELAGYIGLKKQPLTDEKAAENQEYDNVLQRYIETKIALPKGTLRDLYLKTGGKNNFSDAVEDVLEKKKLDFDIELDKKYYDYEIDKRKEAPKDREGLKTARKLRETMAKDRLCLLTGMKFYENIREDLNIRWERRVGKNVIYADIYKKGDKTKKLFTLKFSEKDYVKMYVIDNTDFLRQVWEKFVKGKEGQEVNYHEFYQKGIQKGLGDFQRDVALKVLKFEETVVEKNAGTGLPVSGINNKLSGIIKNRGREMKSDFNKHRDVFVSNDTICKAFFDDADDIERVKKIRNSAFHYNADFEDEDYTGFNKIMDREGIKIKEIDGKKQEGKQRKRF

SEQ ID NO: 58 - protein, 838 aa, unidentified
MQFENIKDTGQKPIYSIDQYEGAKKWCFAIVLNRACDNYEDNPQLFSESLLRFEEVNRRDWFDKDIRDLIKKADTEDQIEPKRKPNTPVNRRLHDIRNYFSHSRHQDDCLYFKNDDPMRCIMEAAYEKAKIHIKGRQTEQSDIPLPELFDANNKITSAGVLFLASFFVERGILHRLMGNIGGFKDNRGKYGLTHDIFTTYCLKDSYSIHASDPKVVLFRDIAGYLSLVACEYYPTYLSKIPKENAGEKSSDEEKYAERKTDKFILFALKYLEEFVLPSLKDDYLVDIGRIDIIREESKETEEKDEQYKPHPNQGKVKVVFDSINKELPYYINHNTVILRIQKNGVMAYSCKIGVNDLKYLLLLCLQGKTDKALDAIYNYLHSMQDPPEVVKIGATDKLFQGLPEFILKQSGIKVQDKNKEKAARIKYIRDKWEKKKSESADMELHRKGRDILRYVNWHCETPLGTEKYDQLLVLLVNKNFVVFGDELNQLKRTEIISKDILEKLSGFQTINTLHQKVCNLVLEELSSLEKNDPGKLAEHIGLVRKPAPENNPPPEYKEKVRRFVEQPMIYKGFLRDQFFVNKDQDGKKLKEQKTFAKLVEETLGQNADVPLGKDFYYVPNIEKDEKKNRFHKDNAVLYETLALDRLCAMMARKCLTQINKNLAEKSEEIDWRNEDGKDFIYLKLVKSDRPQETFKIRFKVNDFAKLYVMDDPDFLGGLMKHFFPQEHSIEYHKLYRNGIERYTDRQKDGIEAILRLEDSVIRQKGMKPKPAKNYISFSEIMAQTDYPEHDQKVLNKVRRAVLHYHLKFEPADYNRFVDIMKKNKFWDGERKNKESRGR

SEQ ID NO: 59 - protein, 790 aa, unidentified
MNGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDDTGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKLDDITKTTVDFKISDKVTVKIPFSNYPSLVYTMSSKYVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 60 - protein, 792 aa, unidentified
MSPDFIKLEKQEAAFYFNQTELNLKAIESNIFDKQQRVILLNNPQILAKVGDFIFNFRDVTKNAKGEIDCLLLKLRELRNFYSHYVYTDDVKILSNGERPLLEKYYQFAIEATGSENVKLEIIESNNRLTEAGVLFFLCMFLKKSQANKLISGISGFKRNDPTGQPRRNLFTYFSVREGYKVVPDMQKHFLLFVLVNHLSGQDDYIEKAQKPYDIGEGLFFHRIASTFLNISGILRNMEFYIYQSKRLKEQQGELKREKDIFPWIEPFQGNSYFEINGNKGIIGEDELKELCYALLVAGKDVRAVEGKITQFLEKFKNADNAQQVEKDEMLDRNNFPANYFAESNIGSIKEKILNRLGKTDDSYNKTGTKIKPYDMMKEVMEFINNSLPADEKLKRKDYRRYLKMVRIWDSEKDNIKREFESKEWSKYFSSDFWMAKNLERVYGLAREKNAELFNKLKAVVEKMDEREFEKYRLINSAEDLASLRRLAKDFGLKWEEKDWQEYSGQIKKQISDRQKLTIMKQRITAELKKKHGIENLNLRITIDSNKSRKAVLNRIAVPRGFVKEHILGWQGSEKVSKKTREAKCKILLSKEYEELSKQFFQTRNYDKMTQVNGLYEKNKLLAFMVVYLMERLNILLNKPTELNELEKAEVDFKISDKVMAKIPFSQYPSLVYAMSSKYADSVGSYKFENDEKNKPFLGKIDTIEKQRMEFIKEVLGFEEYLFEKKIIDKSEFADTATHISFDEICNELIKKGWDKDKLTKLKDARNAALHGEIPAETSFREAKPLINGLKK

SEQ ID NO: 61 - protein, 866 aa, unidentified
MAVDYSLKNEWYREINKSCFTVALNVAYDNCKAKGHENLLREAQRSKGGITNEQIKNVQTEIKTRLEDIRSHFSHFYHDEKSLIFEKDNIVKDFLESAYEKAQSSVIGSTRQSDYKGVVPPLFEPHDGMITAAGVVFLASFFCHRSNVYRMLGAVKGFKHTGKEELSDGAKRDYGFTRRLMAHYSLRDSYVIKAEETKSFRDLLGYLSRVPQQAVDWLNEHNQLSEDEKKEFLNQKPSDEESQEQSKTENTDRQADRMPRRSLRKTDKFILFAAKFIEDWAQKEKMDVTFARYQKTVTEDENKNQDGKQVRDVQLKYEKDTKKLNPDFDYKWTYYIRNNHAIIQIKPDEYKQAVSARISENELKYLVLLIFQGKGWEAIKKIGDYIFHIGNKIKIGRFDHNEERRMPSFLKNPPADIIGEMVENRLKYIRDELNKVIETIKKEEPQNNKWLLYKGKKISIILKFISDSISDIKKRPDVNEYNTLRDMLQKLDFDNFYERLKSYVSEGRIEQTLYDEIKGIKDISTLCIKICELRLAALEELEKEGGDDLNKYIGLAVQEKHKNYDDSNTPQKKAERFLESQFSVGKNFLRETFYDEYIKNRKSLYEIIKEKITGITPLNENRWYLMDKNPKEFESKDSKIIRGLCNIYIQDILCMKIALWYYENLSPSYKNKLKWDFIGQGFGYDRYKLSYKTDCGITIEFKLADLNRLDIIEKPKMIENICHSFILEKDVKKQTISWHEFRQDGIAKYRKLQKEVVEAVFEFENSLKIPDKNWLTQGYVPFNKNKRFEDKGFSTFILEEAVRKGKIKSDDKEPLRKVRTDFFHEQFDSTDAERRIFDKYMPAKHDGKNKGGKMQEKQEKSYTRRI

SEQ ID NO: 62 - protein, 792 aa, unidentified
MSPDFIKLEKQEAAFYFNQTELNLKAIESNILDKQQRMILLNNPRILAKVGNFIFNFRDVTKNAKGEIDCLLFKLEELRNFYSHYVHTDNVKELSNGEKPLLERYYQIAIQATRSEDVKFELFETRNENKITDAGVLFFLCMFLKKSQANKLISGISGFKRNDPTGQPRRNLFTYFSAREGYKALPDMQKHFLLFTLVNYLSNQDEYISELKQYGEIGQGAFFNRIASTFLNISGISGNTKFYSYQSKRIKEQRGELNSEKDSFEWIEPFQGNSYFEINGHKGVIGEDELKELCYALLVAKQDINAVEGKIMQFLKKFRNTGNLQQVKDDEMLEIEYFPASYFNESKKEDIKKEILGRLDKKIRSCSAKAEKAYDKMKEVMEFINNSLPAEEKLKRKDYRRYLKMVRFWSREKGNIEREFRTKEWSKYFSSDFWRKNNLEDVYKLATQKNAELFKNLKAAAEKMGETEFEKYQQINDVKDLASLRRLTQDFGLKWEEKDWEEYSEQIKKQITDRQKLTIMKQRVTAELKKKHGIENLNLRITIDSNKSRKAVLNRIAIPRGFVKKHILGWQGSEKISKNIREAECKILLSKKYEELSRQFFEAGNFDKLTQINGLYEKNKLTAFMSVYLMGRLNIQLNKHTELGNLKKTEVDFKISDKVTEKIPFSQYPSLVYAMSRKYVDNVDKYKFSHQDKKKPFLGKIDSIEKERIEFIKEVLDFEEYLFKNKVIDKSKFSDTATHISFKEICDEMGKKGCNRNKLTELNNARNAALHGEIPSETSFREAKPLINELKK

SEQ ID NO: 63 - protein, 1115 aa, Fusobacterium necrophorum
MEKFRRQNRNSIIKIIISNYDTKGIKELKVRYRKQAQLDTFIIKTEIVNNDIFIKSIIEKAREKYRYSFLFDGEEKYHFKNKSSVEIVKKDIFSQTPDNMIRNYKITLKISEKNPRVVEAEIEDLMNSTILKDGRRSARREKSMTERKLIEEKVAKNYSLLANCPMEEVDSIKIYKIKRFLTYRSNMLLYFASINSFLCEGIKGKDNETEEIWHLKDNDVRKEKVRENFKNKLIQSTENYNSSLKNQIEEKEKLLRKEFKKGAFYRTIIKKLQQERIKELSEKSLTEDCEKIIKLYSKLRHSLMHYDYQYFENLFENKKNDDLMKDLNLDLFKSLPLIRKMKLNNKVNYLEDGDTLFVLQKTKKAKTLYQIYDALCEQKNGFNKFINDFFVSDGEENTVFKQIINEKFQSEMEFLEKRISESEKKNEKLKKKLDSMKAHFRNINSEDTKEAYFWDIHSSRNYKTKYNERKNLVNEYTELLGSSKEKKLLREEITKINRQLLKLKQEMEEITKKNSLFRLEYKMKIAFGFLFCEFDGNISKFKDEFDASNQEKIIQYHKNGEKYLTSFLKEEEKEKFNLEKMQKIIQKTEEEDWLLPETKNNLFKFYLLTYLLLPYELKGDFLGFVKKHYYDIKNVDFIDENQNNIQVSQTVEKQEDYFYHKIRLFEKNTKKYEIVKYSIVPNEKLKQYFEDLGIDIKYLTVEQKSEVSEEKNKKVSLKNNGMFNKTILLFVFKYYQIAFKLFNDIELYSLFFLREKSGKPLEIFRKELESKMKDGYLNFGQLLYVVYEVLVKNKDLDKILSKKIDYRKDKSFSPEIAYLRNFLSHLNYSKFLDNFMKINTNKSDENKEVLIPSIKIQKMIQFIEKCNLQNQIDFDFNFVNDFYMRKEKMFFIQLKQIFPDINSTEKQKMNEKEEILRNRYHLTDKKNEQIKDEHEAQSQLYEKILSLQKIYSSDKNNFYGRLKEEKLLFLEKQGKKKLSMEEIKDKIAGDISDLLGILKKEITRDIKDKLTEKFRYCEEKLLNLSFYNHQDKKKEESIRVFLIRDKNSDNFKFESILDDGSNKIFISKNGKEITIQCCDKVLETLIIEKNTLKISSNGKIISLIPHYSYSIDVKY

SEQ ID NO: 64 - protein, 1111 aa, Fusobacterium necrophorum
MEKFRRQNRSSIIKIIISNYDTKGIKELKVRYRKQAQLDTFIIKTEIVNNDIFIKSIIEKAREKYRYSFLFDGEEKYHFKNKSSVEIVKKDIFSQTPDNMIRNYKITLKISEKNPRVVEAEIEDLMNSTILKDGRRSARREKSMTERKLIEEKVAENYSLLANCPMEEVDSIKIYKIKRFLTYRSNMLLYFASINSFLCEGIKGKDNETEEIWHLKDNDVRKEKVKENFKNKLIQSTENYNSSLKNQIEEKEKLLRKESKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEIEFLEKRISESEKKNEKLKKKLDSMKAHFRNINSEDTKEAYFWDIHSSRNYKTKYNERKNLVNEYTELLGSSKEKKLLREEITKINRQLLKLKQEMEEITKKNSLFRLEYKMKMAFGFLFCEFDGNISRFKDEFDASNQEKIIQYHKNGEKYLTYFLKEEEKEKFNLKKLQETIQKTGEENWLLPQNKNNLFKFYLLTYLLLPYELKGDFLGFVKKHYYDIKNVDFMDENQSSKIIESKEDDFYHKIRLFEKNTKKYEIVKYSIVPDKKLKQYFKDLGIDTKYLILDQKSEVSGEKNKKVSLKNNGMFNKTILLFVFKYYQIAFKLFNDIELYSLFFLREKSGKPFEVFLKELKDKMIGKQLNFGQLLYVVYEVLVKNKDLSEILSERIDYRKDMCFSAEIADLRNFLSHLNYSKFLDNFMKINTNKSDENKEVLIPSIKIQKMIKFIEECNLQSQIDFDFNFVNDFYMRKEKMFFIQLKQIFPDINSTEKQKMNEKEEILRNRYHLTDKKNEQIKDEHEAQSQLYEKILSLQKIYSSDKNNFYGRLKEEKLLFLEKQEKKKLSMEEIKDKIAGDISDLLGILKKEITRDIKDKLTEKFRYCEEKLLNLSFYNHQDKKKEESIRVFLIRDKNSDNFKFESILDDGSNKIFISKNGKEITIQCCDKVLETLIIEKNTLKISSNGKIISLIPHYSYSIDVKY

SEQ ID NO: 65 - protein, 1110 aa, Fusobacterium necrophorum
MKVRYRKQAQLDTFIIKTEIVNNDIFIKSIIEKAREKYRYSFLFDGEEKYHFKNKSSVEIVKNDIFSQTPDNMIRNYKITLKISEKNPRVVEAEIEDLMNSTILKDGRRSARREKSMTERKLIEEKVAENYSLLANCPIEEVDSIKIYKIKRFLTYRSNMLLYFASINSFLCEGIKGKDNETEEIWHLKDNDVRKEKVKENFKNKLIQSTENYNSSLKNQIEEKEKLSSKEFKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKLDSMKAHFRNINSEDTKEAYFWDIHSSRNYKTKYNERKNLVNEYTKLLGSSKEKKLLREEITKINRQLLKLKQEMEEITKKNSLFRLEYKMKIAFGFLFCEFDGNISKFKDEFDASNQEKIIQYHKNGEKYLTSFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKNKKVSLKNNGMFNKTILLFVFKYYQIAFKLFNDIELYSLFFLREKSEKPFEVFLEELKDKMIGKQLNFGQLLYVVYEVLVKNKDLDKILSKKIDYRKDKSFSPEIAYLRNFLSHLNYSKFLDNFMKINTNKSDENKEVLIPSIKIQKMIQFIEKCNLQNQIDFDFNFVNDFYMRKEKMFFIQLKQIFPDINSTEKQKKSEKEEILRKRYHLINKKNEQIKDEHEAQSQLYEKILSLQKIFSCDKNNFYRRLKEEKLLFLEKQGKKKISMKEIKDKIASDISDLLGILKKEITRDIKDKLTEKFRYCEEKLLNISFYNHQDKKKEEGIRVFLIRDKNSDNFKFESILDDGSNKIFISKNGKEITIQCCDKVLETLMIEKNTLKISSNGKIISLIPHYSYSIDVKY

SEQ ID NO: 66 - protein, 668 aa, Fusobacterium necrophorum
MTEKKSIIFKNKSSVEIVKKDIFSQTPDNMIRNYKITLKISEKNPRVVEAEIEDLMNSTILKDGRRSARREKSMTERKLIEEKVAENYSLLANCPMEEVDSIKIYKIKRFLTYRSNMLLYFASINSFLCEGIKGKDNETEEIWHLKDNDVRKEKVKENFKNKLIQSTENYNSSLKNQIEEKEKLLRKESKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKFDSMKAHFHNINSEDTKEAYFWDIHSSSNYKTKYNERKNLVNEYTELLGSSKEKKLLREEITQINRKLLKLKQEMEEITKKNSLFRLEYKMKIAFGFLFCEFDGNISKFKDEFDASNQEKIIQYHKNGEKYLTYFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKIKKFL

SEQ ID NO: 67 - protein, 1121 aa, Fusobacterium perfoetens
MGKPNRSSIIKIIISNYDNKGIKEVKVRYNKQAQLDTFLIKSELKDGKFILYSIVDKAREKYRYSFEIDKTNINKNEILIIKKDIYSNKEDKVIRKYILSFEVSEKNDRTIVTKIKDCLETQKKEKFERENTRRLISETERKLLSEETQKTYSKIACCSPEDIDSVKIYKIKRYLAYRSNMLLFFSLINDIFVKGVVKDNGEEVGEIWRIIDSKEIDEKKTYDLLVENFKKRMSQEFINYKQSIENKIEKNTNKIKEIEQKLKKEKYKKEINRLKKQLIELNRENDLLEKDKIELSDEEIREDIEKILKIYSDLRHKLMHYNYQYFENLFENKKISKEKNEDVNLTELLDLNLFRYLPLVRQLKLENKTNYLEKEDKITVLGVSDSAIKYYSYYNFLCEQKNGFNNFINSFFSNDGEENKSFKEKINLSLEKEIEIMEKETNEKIKEINKNELQLMKEQKELGTAYVLDIHSLNDYKISHNERNKNVKLQNDIMNGNRDKNALDKINKKLVELKIKMDKITKRNSILRLKYKLQVAYGFLMEEYKGNIKKFKDEFDISKEKIKSYKSKGEKYLEVKSEKKYITKILNSIEDIHNITWLKNQEENNLFKFYVLTYILLPFEFRGDFLGFVKKHYYDIKNVEFLDENNDRLTPEQLEKMKNDSFFNKIRLFEKNSKKYDILKESILTSERIGKYFSLLNTGAKYFEYGGEENRGIFNKNIIIPIFKYYQIVLKLYNDVELAMLLTLSESDEKDINKIKELVTLKEKVSPKKIDYEKKYKFSVLLDCFNRIINLGKKDFLASEEVKEVAKTFTNLAYLRNKICHLNYSKFIDDLLTIDTNKSTTDSEGKLLINDRIRKLIKFIRENNQKMNISIDYNYINDYYMKKEKFIFGQRKQAKTIIDSGKKANKRNKAEELLKMYRVKKENINLIYELSKKLNELTKSELFLLDKKLLKDIDFTDVKIKNKSFFELKNDVKEVANIKQALQKHSSELIGIYKKEVIMAIKRSIVSKLIYDEEKVLSIIIYDKTNKKYEDFLLEIRRERDINKFQFLIDEKKEKLGYEKIIETKEKKKVVVKIQNNSELVSEPRIIKNKDKKKAKTPEEISKLGILDLTNHYCFNLKITL

SEQ ID NO: 68 - protein, 897 aa, Fusobacterium ulcerans
MENKGNNKKIDFDENYNILVAQIKEYFTKEIENYNNRIDNIIDKKELLKYSEKKEESEKNKKLEELNKLKSQKLKILTDEEIKADVIKIIKIFSDLRHSLMHYEYKYFENLFENKKNEELAELLNLNLFKNLTLLRQMKIENKTNYLEGREEFNIIGKNIKAKEVLGHYNLLAEQKNGFNNFINSFFVQDGTENLEFKKLIDEHFVNAKKRLERNIKKSKKLEKELEKMEQHYQRLNCAYVWDIHTSTTYKKLYNKRKSLIEEYNKQINEIKDKEVITAINVELLRIKKEMEEITKSNSLFRLKYKMQIAYAFLEIEFGGNIAKFKDEFDCSKMEEVQKYLKKGVKYLKYYKDKEAQKNYEFPFEEIFENKDTHNEEWLENTSENNLFKFYILTYLLLPMEFKGDFLGVVKKHYYDIKNVDFTDESEKELSQVQLDKMIGDSFFHKIRLFEKNTKRYEIIKYSILTSDEIKRYFRLLELDVPYFEYEKGTDEIGIFNKNIILTIFKYYQIIFRLYNDLEIHGLFNISSDLDKILRDLKSYGNKNINFREFLYVIKQNNNSSTEEEYRKIWENLEAKYLRLHLLTPEKEEIKTKTKEELEKLNEISNLRNGICHLNYKEIIEEILKTEISEKNKEATLNEKIRKVINFIKENELDKVELGFNFINDFFMKKEQFMFGQIKQVKEGNSDSITTERERKEKNNKKLKETYELNCDNLSEFYETSNNLRERANSSSLLEDSAFLKKIGLYKVKNNKVNSKVKDEEKRIENIKRKLLKDSSDIMGMYKAEVVKKLKEKLILIFKHDEEKRIYVTVYDTSKAVPENISKEILVKRNNSKEEYFFEDNNKKYVTEYYTLEITETNELKVIPAKKLEGKEFKTEKNKENKLMLNNHYCFNVKIIY

SEQ ID NO: 69 - protein, 1111 aa, Anaerosalibacter massiliensis
MKSGRREKAKSNKSSIVRVIISNFDDKQVKEIKVLYTKQGGIDVIKFKSTEKDEKGRMKFNFDCAYNRLEEEEFNSFGGKGKQSFFVTTNEDLTELHVTKRHKTTGEIIKDYTIQGKYTPIKQDRTKVTVSITDNKDHFDSNDLGDKIRLSRSLTQYTNRILLDADVMKNYREIVCSDSEKVDETINIDSQEIYKINRFLSYRSNMIIYYQMINNFLLHYDGEEDKGGNDSINLINEIWKYENKKNDEKEKIIERSYKSIEKSINQYILNHNTEVESGDKEKKIDISEERIKEDLKKTFILFSRLRHYMVHYNYKFYENLYSGKNFIIYNKDKSKSRRFSELLDLNIFKELSKIKLVKNRAVSNYLDKKTTIHVLNKNINAIKLLDIYRDICETKNGFNNFINNMMTISGEEDKEYKEMVTKHFNENMNKLSIYLENFKKHSDFKTNNKKKETYNLLKQELDEQKKLRLWFNAPYVYDIHSSKKYKELYVERKKYVDIHSKLIEAGINNDNKKKLNEINVKLCELNTEMKEMTKLNSKYRLQYKLQLAFGFILEEFNLDIDKFVSAFDKDNNLTISKFMEKRETYLSKSLDRRDNRFKKLIKDYKFRDTEDIFCSDRENNLVKLYILMYILLPVEIRGDFLGFVKKNYYDLKHVDFIDKRNNDNKDTFFHDLRLFEKNVKRLEVTSYSLSDGFLGKKSREKFGKELEKFIYKNVSIALPTNIDIKEFNKSLVLPMMKNYQIIFKLLNDIEISALFLIAKKEGNEGSITFKKVIDKVRKEDMNGNINFSQVMKMALNEKVNCQIRNSIAHINMKQLYIEPLNIYINNNQNKKTISEQMEEIIDICITKGLTGKELNKNIINDYYMKKEKLVFNLKLRKRNNLVSIDAQQKNMKEKSILNKYDLNYKDENLNIKEIILKVNDLNNKQKLLKETTEGESNYKNALSKDILLLNGIIRKNINFKIKEMILGIIQQNEYRYVNINIYDKIRKEDHNIDLKINNKYIEISCYENKSNESTDERINFKIKYMDLKVKNELLVPSCYEDIYIKKKIDLEIRYIENCKVVYIDIYYKKYNINLEFDGKTLFVKFNKDVKKNNQKVNLESNYIQNIKFIVS

SEQ ID NO: 70 - protein, 7 aa, synthetic construct
PKKKRRV

SEQ ID NO: 71 - protein, 16 aa, synthetic construct
KRPAATKKAGQAKKKK

SEQ ID NO: 72 - protein, 13 aa, synthetic construct
GCCGCCRCCAUGG

SEQ ID NO: 73 - RNA, 21 nt, synthetic construct
  source 1..21: mol_type = other RNA
gcagcagcagcagcagcagca

SEQ ID NO: 74 - RNA, 21 nt, synthetic construct
  source 1..21: mol_type = other RNA
gcaggcaggcaggcaggcagg

SEQ ID NO: 75 - RNA, 21 nt, synthetic construct
  source 1..21: mol_type = other RNA
gcagcagcagcagcagcagca

SEQ ID NO: 76 - RNA, 21 nt, synthetic construct
  source 1..21: mol_type = other RNA
ggccccggcccggccccggcc

SEQ ID NO: 77 - RNA, 21 nt, synthetic construct
  source 1..21: mol_type = other RNA
gccgccgccgccgccgccgcc

SEQ ID NO: 78 - RNA, 21 nt, synthetic construct
  source 1..21: mol_type = other RNA
gcccaggcccaggcccaggcc

SEQ ID NO: 79 - RNA, 21 nt, synthetic construct
  source 1..21: mol_type = other RNA
gttaggttaggttaggttagg

SEQ ID NO: 80 - RNA, 21 nt, synthetic construct
  source 1..21: mol_type = other RNA
gaatagaatagaatagaatag

SEQ ID NO: 81 - DNA, 12 bp, Homo sapiens
  source 1..12: mol_type = genomic DNA
ccccgccccgcg

SEQ ID NO: 82 - protein, 6 aa, synthetic construct
HHHHHH